(19) United States
(12) Patent Application Publication
(10) Pub. No.: US 2008/0096207 A1
(43) Pub. Date: Apr. 24, 2008

(54) Leptin and Growth Hormone Receptor Gene Markers Associated with Rearing, Carcass Traits and Productive Life in Cattle

(76) Inventor: Brent Woodward, Lawrenceville, GA (US)

Correspondence Address: FROMMER LAWRENCE & HAUG, 745 FIFTH AVENUE- 10TH FL., NEW YORK, NY, 10151, US

(21) Appl. No.: 11/837,221

(22) Filed: Aug. 10, 2007

Related U.S. Application Data
(60) Provisional application No. 60/836,854, filed on Aug. 10, 2006.

(51) Int. Cl. C07H 21/04; C12Q 1/68

(52) U.S. Cl. 435/6; 536/24.31

(57) ABSTRACT

The invention provides a method for sub-grouping animals according to genotype wherein the animals of each sub-group have a similar polymorphism or combination of polymorphisms in the leptin gene selected from the group consisting of UASMS1, UASMS2, UASMS3, EXON2-FB, and E2JW. The combination of single nucleotide polymorphisms of the leptin gene, especially combinations which may comprise alleles of the E2JW locus, may indicate an increase in the tenderness of meat as well as indicating the quality of other traits of the animals. The leptin polymorphisms may also be combined with polymorphisms of the bovine growth hormone receptor gene. The invention also provides methods for identifying an animal having a desirable phenotype relating to certain feed intake, dry material intake, growth rate, body weight, carcass merit and composition, and milk yield, as compared to the general population of animals of that species, which may comprise determining the presence of a single nucleotide polymorphism or combination of single nucleotide polymorphisms in the leptin and/or bGHr genes.

### RELATED APPLICATIONS/PATENTS & INCORPORATION BY REFERENCE

This application claims priority to U.S. Provisional Patent Application No. 60/836,854 filed Aug. 10, 2006. Reference is made to U.S. application Ser. No. 11/366,069 filed Mar. 2, 2006.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

More generally, documents or references are cited in this text, either in a Reference List before the claims, or in the text itself, and, each of these documents or references (“herein cited references”), as well as each document or reference cited in each of the herein-cited references (including any manufacturer's specifications, instructions, etc.), is hereby expressly incorporated herein by reference.

### FIELD OF THE INVENTION

The present invention relates to single nucleotide polymorphisms in the leptin or ob gene, and to the association of these SNPs alone or in combination, or in combination with SNPs of other genes, with certain traits that are economically important in livestock species, such as circulating leptin levels, feed intake, growth rate, body weight, carcass merit, meat tenderness and carcass composition, ribeye area, yield grade and dry matter intake.

### BACKGROUND OF THE INVENTION

Significant improvements in animal performance, efficiency and carcass and meat quality have been made over the years through the application of standard animal breeding and selection techniques. However, such classical animal breeding techniques require several years of genetic evaluation of performance records on individual animals and their relatives and are therefore very expensive. Other efforts have been made to improve productivity and quality through the application of such management practices as the use of feed additives, animal hormonal implants and chemotherapeutics. However, there is significant political and regulatory resistance to the introduction and use of such methodologies. Such methodologies are also non-inheritable and need to be applied differently in every production system.

There is a need for methods that allow relatively easy and more efficient selection and breeding of farm animals with an advantage for an inheritable trait of circulating leptin levels, feed intake, growth rate, body weight, carcass merit and carcass composition. The economic significance of the use of genetic markers that are associated with specific economically important traits (especially traits with low heritability) in livestock through marker-assisted selection and/or management cannot therefore be over-emphasized.

Leptin, the hormone product of the ob (obese) gene, has been shown to be predominantly synthesized and expressed in adipose tissues (Zhang et al., Nature. 1994 Dec. 1; 372(6505):425-32, Ji et al., Anim Biotechnol. 1998; 9(1):1-14). It functions as a potent physiological signal in the regulation of body weight, energy expenditure, feed intake, adiposity, fertility and immune functions (Houseknecht et al., J Anim Sci. 1998 May; 76(5):1405-20, Lord et al., Nature. 1998 Aug. 27; 394(6696):897-901, Garcia et al., J Anim Sci. 2002 August; 80(8):2158-67). Leptin has been proposed as one of the major control factors contributing to the phenotypic and genetic variation in the performance and efficiency of cattle.

Polymorphisms in the coding regions of the leptin gene in cattle have been associated with milk yield and composition (Liefers et al., J Dairy Sci. 2002 June; 85(6):1633-8), feed intake (Liefers et al., J Dairy Sci. 2002 June; 85(6):1633-8; Lagonigro et al., Anim Genet. 2003 October; 34(5):371-4), and body fat (Buchanan et al., Genet Sel Evol. 2002 January-February; 34(1):105-16; Lagonigro et al., Anim Genet. 2003 October; 34(5):371-4). However, it would appear that polymorphisms located in the promoter region of the leptin gene (i.e. the region of the gene that regulates the level of leptin expression through its associated enhancer and silencer elements) may have a stronger effect on the regulation of these economically important traits, and therefore be of greater predictive value.

Studies in humans for instance, have shown that mutations in the CCAAT/enhancer binding protein (C/EBP-α) region of the leptin promoter abolished inducibility of the promoter by C/EBP-α (Miller et al., Proc Natl Acad Sci USA. 1996 May 28; 93(11):5507-11). Mason et al. (Endocrinology. 1998 March; 139(3):1013-22) have shown that mutations in the C/EBP-α and TATA motifs as well as in a consensus Sp1 site of leptin reduced promoter activity by 10, 10 and 2.5-fold, respectively, and abolished binding of these factors. Mason et al. (Endocrinology. 1998 March; 139(3):1013-22) also showed that the regulation of leptin gene expression is partly linked to a novel factor that binds to an LP1 motif in the promoter. The role of peroxizome proliferator activated receptor-γ (PPAR-γ) in adipocyte differentiation has also been linked to leptin promoter function (De Vos et al., J Clin Invest. 1996 Aug. 15; 98(4):1004-9.). Though several polymorphisms have been detected in the bovine leptin promoter (Liefers et al., Mamm Genome. 2003 September; 14(9):657-63), little has been done to associate any of these with any economically important traits in cattle.

SNPs of other genes of Quantitative Gene Loci (QTL) are also associated with economically significant traits of cattle such as meat quality or milk yield. One SNP, in exon 8 of the gene encoding the bovine growth hormone receptor (bGHr), has been shown to influence milk yield and composition (Blott et al., Genetics 163: 253-266 (2003).

In the present invention it has surprisingly been shown that three previously unknown single nucleotide polymorphisms (SNPs) in the promoter region of the leptin gene, alone or in combination with SNPs in exon 2 of the leptin gene are strongly associated with several economically important traits in cattle. In addition, the present invention has shown that an SNP in exon 8 of the bGHr locus is quantitatively associated with meat quality and the daily feed intake of the animals and, therefore, provides a further useful marker in beef as well as in dairy cattle.

Citation or identification of any document in this application is not an admission that such document is available as prior art to the present invention.

### SUMMARY OF THE INVENTION

The present invention relates generally to three previously unknown single nucleotide polymorphisms (SNPs) in the promoter of the leptin or ob gene (SEQ ID NO: 1), to two previously known SNPs in exon 2 of ob gene (SEQ ID NO: 5), to other SNPs, particularly the bovine growth hormone receptor (bGHr) gene, and to the association of each of these SNPs, alone or in combination, with certain traits that are of significant economic importance in livestock species, such as circulating leptin levels, daily feed intake, growth rate, body weight, carcass merit and carcass composition in livestock species. The three SNPs located in the leptin gene promoter are named UASMS1, UASMS2, and UASMS3. These three SNPs, in the context of the ob gene promoter sequence, can be seen in SEQ ID NO: 2, SEQ ID NO: 3 and SEQ ID NO: 4, respectively. The SNPs located in exon 2 of the leptin gene is named EXON2-FB, seen in the context of exon 2 of the ob gene in SEQ ID NO: 6.

In one aspect, the present invention provides methods for grouping animals according to genotype wherein the animals of each sub-group may have a similar polymorphism in the leptin gene. The present invention may also encompass grouping the animals according to SNPs of other genetic loci, and in combination with one or more SNPs associated with the leptin gene. Such methods may comprise determining the genotype of each animal to be subgrouped by determining the presence of a SNP in the leptin gene, wherein the SNP is selected from the group consisting of UASMS1, UASMS2, UASMS3, E2JW and EXON2-FB, or in such as the bGHr gene locus (for example SNP F279Y), and wherein individual animals are placed into sub-groups where each animal in a subgroup has a similar polymorphisms in the selected genes. In a preferred embodiment the animal to be grouped is a bovine, and the leptin gene is the bovine leptin gene.

In another embodiment, the present invention provides methods for identifying animals having desirable traits relating to circulating leptin levels, daily feed intake, growth rate, body weight, carcass merit and carcass composition, as compared to the general population of animals of that species. Such methods may comprise determining the presence of SNPs in the leptin or other relevant genes of the animal that may provide prediction of desirable traits of cattle, wherein the leptin polymorphisms may be selected from the group consisting of UASMS1, UASMS2, UASMS3, E2JW, EXON2-FB, and bGHr F279Y, and wherein the presence of the UASMS1, UASMS2, UASMS3, E2JW, EXON2-FB, or bGHr F279Y SNP is indicative of a desirable trait relating to circulating leptin levels, feed intake, growth rate, body weight, carcass merit and carcass composition. In a preferred embodiment the animal to be grouped is a bovine, and the leptin gene is the bovine leptin gene.

In a further embodiment the present invention provides isolated oligonucleotide probes that are useful in the detection of the UASMS1, UASMS2, UASMS3, E2JW and EXON2-FB SNPs in the ob gene. The present invention advantageously provides oligonucleotide probes for detection of the two alternative alleles of each SNP. For example, in the case of the UASMS1 polymorphism, which constitutes a C to T substitution at nucleotide position 207 of the ob gene promoter, the present invention provides oligonucleotide probes that can be used to detect and distinguish between the C-containing allele and the T-containing allele. In the case of the UASMS2 polymorphism, which constitutes a C to T substitution at nucleotide position 528 of the ob gene promoter, the present invention provides oligonucleotide probes that can be used to detect and distinguish between the C-containing allele and the T-containing allele. In the case of the UASMS3 polymorphism, which constitutes a C to G substitution at nucleotide position 1759 of the ob gene promoter, the present invention provides oligonucleotide probes that can be used to detect and distinguish between the C-containing allele and the G-containing allele. Similarly, in the case of the EXON2-FB polymorphism, which constitutes a C to T substitution at nucleotide position 305 of exon 2 of the ob gene, the present invention provides oligonucleotide probes that can be used to detect and distinguish between the C-containing allele and the T-containing allele. In the case of the bGHr F279Y SNP, which results in an F to Y substitution at amino acid position 279 within exon 8 of the bGHr gene, the present invention provides oligonucleotide probes that can be used to detect and distinguish between the respective alleles. In a preferred embodiment, the oligonucleotide probes of the present invention are labeled with a detectable moiety, such as for example, digoxigenin-dUTP, biotin, fluorescent moieties, chemiluminescent moieties, electrochemiluminescent moieties and radioactive moieties.

In a further embodiment the present invention provides isolated primers and primer pairs that are useful in the amplification of fragments of the ob gene that span the UASMS1, UASMS2, UASMS3, E2JW, EXON2-FB, and bGHr F279Y SNPs. In one embodiment fragments of the ob gene that are amplified using such primers are subsequently detected using the oligonucloetide probes of the present invention.

One aspect of the invention, therefore, provides a method for sub grouping animals according to genotype wherein the animals of each sub-group have a similar polymorphism or combination of polymorphisms in the leptin gene comprising (a) determining the genotype of each animal to be subgrouped by determining the presence of a single nucleotide polymorphism or a combination of single nucleotide polymorphisms in the leptin (ob) gene, wherein the single nucleotide polymorphisms are selected from the group consisting of UASMS1, UASMS2, UASMS3, EXON2-FB, and E2JW; and segregating individual animals into sub-groups wherein each animal in a subgroup has a similar polymorphism or combination of polymorphisms in the leptin gene.

In one embodiment of the invention, the method may further sub-group the animals according to the genotype for the bGHr gene, and in particular that relating to the F279Y SNP. In various embodiments of this aspect of the invention, the combination of single nucleotide polymorphisms of the leptin gene may be selected from the group consisting of UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, and UASMS3/E2JW, and wherein individual animals are segregated into sub-groups depending on whether the animals have, or do not have, the UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, and UASMS3/E2JW single nucleotide polymorphism combinations of the leptin gene.

In one embodiment, the combination of single nucleotide polymorphisms of the leptin gene comprises the markers UASMS1/EXON2-FB, UASMS3/EXON2-FB, EXON2-FB/E2JW, UASMS1/E2JW, or UASMS3/E2JW, and wherein the combination of SNPs indicates an increase in the tenderness of meat.

In another embodiment according to the invention, the combination of single nucleotide polymorphisms of the leptin gene comprises the markers UASMS1/EXON2-FB, UASMS3/EXON2-FB, EXON2-FB/E2JW, UASMS1/E2JW, or UASMS3/E2JW.

In still another embodiment of the invention, the combination of single nucleotide polymorphisms of the leptin gene comprises the markers EXON2-FB/E2JW.

In other embodiments of the invention, the combination of single nucleotide polymorphisms of the leptin gene comprises the markers UASMS1/EXON2-FB or UASMS3/EXON2-FB.

In still other embodiments of the invention, the combination of single nucleotide polymorphisms of the leptin gene comprises the markers UASMS1/E2JW, or UASMS3/E2JW.

In these embodiments of the invention, the single or combined SNPs of the leptin gene may also be combined with the F279Y SNP of the bGHr gene.

Another aspect of the invention provides a method for identifying an animal having a desirable phenotype relating to certain feed intake, growth rate, body weight, carcass merit and composition, and milk yield, as compared to the general population of animals of that species, comprising determining the genotype of the animal, wherein the single nucleotide polymorphisms are selected from the group consisting of UASMS1, UASMS2, UASMS3, EXON2-FB, E2JW and F279Y, and wherein the combination of single nucleotide polymorphisms is selected from the group consisting of UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, and UASMS3/E2JW, UASMS1/UASMS2/F279Y, UASMS1/UASMS3/F279Y, UASMS2/UASMS3/F279Y, UASMS1/EXON2-FB/F279Y, UASMS2/EXON2-FB/F279Y UASMS3/EXON2-FB/F279Y, EXON2-FB/E2JW/F279Y, UASMS1/E2JW/F279Y, UASMS2/E2JW/F279Y, and UASMS3/E2JW/F279Y and wherein the presence of either the UASMS1, UASMS2, UASMS3 or EXON2-FB single nucleotide polymorphism or the presence of the UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, UASMS3/E2JW, UASMS1/UASMS2/F279Y, UASMS1/UASMS3/F279Y, UASMS2/UASMS3/F279Y, UASMS1/EXON2-FB/F279Y, UASMS2/EXON2-FB/F279Y UASMS3/EXON2-FB/F279Y, EXON2-FB/E2JW/F279Y, UASMS1/E2JW/F279Y, UASMS2/E2JW/F279Y, or UASMS3/E2JW/F279Y combinations of single nucleotide polymorphisms is indicative of a desirable phenotype relating to certain feed intake, growth rate, body weight, carcass merit and composition, meat quality, meat tenderness or and milk yield.

Still another aspect of the invention provides a composition for the detection of a combination of ob gene polymorphisms selected from the group consisting of UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, UASMS3/E2JW, UASMS1/UASMS2/F279Y, UASMS1/UASMS3/F279Y, UASMS2/UASMS3/F279Y, UASMS1/EXON2-FB/F279Y, UASMS2/EXON2-FB/F279Y UASMS3/EXON2-FB/F279Y, EXON2-FB/E2JW/F279Y, UASMS1/E2JW/F279Y, UASMS2/E2JW/F279Y, or UASMS3/E2JW/F279Y, comprising at least two oligonucleotide probes, wherein each oligonucleotide probe is capable of selectively detecting a single polymorphism, and wherein each probe is optionally labeled with a detectable moiety.

One embodiment of this aspect of the invention is an isolated oligonucleotide probe, wherein the detectable moiety is selected from the group consisting of a radiolabel ³H, ¹²⁵I, ³⁵S, ¹⁴C, ³²P, a detectable enzyme, horse radish peroxidase (HRP), alkaline phosphatase, a fluorescent dye, fluorescein isothiocyanate, Texas red, rhodamine, Cy3, Cy5, Bodipy, Bodipy Far Red, Lucifer Yellow, Bodipy 630/650-X, Bodipy R6G-X, 5-CR 6G, a colorimetric label, colloidal gold digoxigenin-dUTP, or biotin. In one embodiment of the invention, the oligonucleotide is immobilized on a solid support.

Still another aspect of the invention provides a method of determining the genotype of an animal at a polymorphic locus of the ob gene comprising: (a) obtaining a DNA sample from the animal; (b) contacting the DNA sample with at least two oligonucleotide primer pairs under conditions suitable for permitting hybridization of the oligonucleotide primers to the DNA sample; (c) enzymatically amplifying specific regions of the ob gene using the primer pairs to form at least two nucleic acid amplification products; (d) contacting the amplification products from step c) with labeled ob gene allele-specific probes, labeled with a detectable moiety, under conditions suitable for permitting hybridization of the labeled allele-specific probes to the amplification products; e) detecting the presence of the amplification products by detecting the detectable moiety of the labeled allele-specific probes hybridized to the amplification products.

In various embodiments of this aspect of the invention, the oligonucleotide primer pairs may be selected from the group consisting of SEQ ID NO: 7 and SEQ ID NO: 8, SEQ ID NO: 11 and SEQ ID NO: 12, SEQ ID NO: 15 and SEQ ID NO: 16, SEQ ID NO: 19 and SEQ ID NO: 20 and primers capable of allowing amplification of a region of the ob gene spanning an E2JW polymorphic locus.

In other embodiments of this method of the invention, the oligonucleotide primer pairs are capable of amplifying regions of a bovine gene having at least one polymorphic nucleotide locus selected from the group consisting of UASMS1, UASMS2, UASMS3, EXON2-FB, and E2JW, or combinations thereof selected from the group consisting of UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, UASMS3/E2JW.

In other embodiments of the invention, the oligonucleotide primer pairs are capable of amplifying regions of a bovine growth hormone gene having at least one polymorphic nucleotide locus such as the F279Y SNP.

In other embodiments, the genotype indicates an increase in the tenderness of bovine meat.

The oligonucleotide probes and primers described herein are useful for identifying animals having SNPs associated with desirable traits relating to circulating leptin levels, feed intake, growth rate, body weight, carcass merit and carcass composition, as compared to the general population of animals of that species. Once individual animals possessing these SNPs have been identified, the animals can then be grouped according to genotype, wherein the animals of each sub-group have a similar polymorphism in the leptin gene. The present invention also advantageously provides compositions and kits comprising the oligonucleotide probes and primers described herein.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 illustrates the nucleotide sequence for the 5′ flanking promoter region and exon 1 of the “wild type” bovine ob gene. This “wild type” sequence has GenBank accession number AB070368 (Taniguchi et al. IUBMB Life Vol 53, p 131-135 (2002)), and is designated herein as SEQ ID NO: 1.

FIG. 2 illustrates the nucleotide sequence the UASMS1 single nucleotide polymorphism in the bovine ob gene promoter (SEQ ID NO: 2). This polymorphic sequence differs from that of the “wild type” bovine ob gene sequence (SEQ ID NO: 1) in that nucleotide position 207 has a cytosine to thymine substitution.

FIG. 3 illustrates the nucleotide sequence the UASMS2 single nucleotide polymorphism of the bovine ob gene (SEQ ID NO: 3). This polymophic sequence differs from that of the “wild type” bovine ob gene sequence (SEQ ID NO: 1) in that nucleotide position 528 has a cytosine to thymine substitution.

FIG. 4 illustrates the nucleotide sequence the UASMS3 single nucleotide polymorphism of the bovine ob gene (SEQ ID NO: 4). This polymorphic sequence differs from that of the “wild type” bovine ob gene sequence (SEQ ID NO: 1) in that nucleotide position 1759 has a cytosine to guanine substitution.

FIG. 5 illustrates the nucleotide sequence for the exon 2 of the “wild type” bovine ob gene (SEQ ID NO: 5). This “wild type” exon 2 sequence has GenBank accession number AY138588.

FIG. 6 illustrates the nucleotide sequence for the EXON2-FB single nucleotide polymorphism of the bovine ob gene (SEQ ID NO: 6). This polymorphic sequence differs from that of the “wild type” bovine ob gene sequence (SEQ ID NO: 5) in that nucleotide position 305 has a cytosine to thymine substitution.

FIG. 7 illustrates using a flow chart how the animals may be screened for the UASMS1 SNP, and how the genotype information may be used to select animals to breed from and/or use for food production.

FIG. 8 illustrates using a flow chart how the animals may be screened for the UASMS2 SNP, and how the genotype information may be used to select animals to breed from and/or use for food production.

FIG. 9 illustrates using a flow chart how the animals may be screened for the UASMS3 SNP, and how the genotype information may be used to select animals to breed from and/or use for food production.

FIG. 10 illustrates using a flow chart how the animals may be screened for the EXON2-FB SNP, and how the genotype information may be used to select animals to breed from and/or use for food production.

FIG. 11 illustrates marker genotype and descriptive statistics for leptin gene SNPs among a population of test cattle.

FIG. 12 illustrates estimations of single marker genotype effects.

FIG. 13 illustrates F-test results of genotype effects.

FIG. 14 illustrates genotype effcts of UASMS2 and EXON2-FB SNPs and genotype frequencies for UASMS2 and EXON2-FB.

FIG. 15 illustrates a regression analysis of the number of alleles.

FIG. 16 illustrates the regression on haplotype frequency of UASMS1 and UASMS2.

FIG. 17 illustrates the regression on haplotype frequency of UASMS1 and EXON2-FB.

FIG. 18 illustrates the regression on haplotype frequency of UASMS2 and EXON2-FB.

FIG. 19 illustrates regression on haplotype frequency of UASMS1, UASMS2 and EXON2-FB.

FIG. 20 illustrates a summary of significant coefficients for beef traits regressed by haplotype frequencies.

FIG. 21 illustrates the association of carcass and performance traits with SNPs of the leptin locus.

FIG. 22 illustrates the nucleotide sequence SEQ ID NO: 21 for the exon 2 of the bovine ob gene. This E2JW SNP of exon 2 sequence has GenBank accession number AY138588.

FIG. 23 illustrates using a flow chart how the animals may be screened for the EXON2-FB/E2JW SNP combination, and how the genotype information may be used to select animals to breed from and/or use for food production with increased meat tenderness.

FIG. 24 illustrates the cDNA sequence of B. taurus growth hormone receptor (SEQ ID NO: 24) (GenBank Accession No. X70041), wherein the start codon is at nucleotide position 19 and the F279Y SNP is at position 854 (Blott et al., 2003).

### DETAILED DESCRIPTION

The term “animal” is used herein to include all vertebrate animals, including humans. It also includes an individual animal in all stages of development, including embryonic and fetal stages. As used herein, the term “production animals” is used interchangeably with “livestock animals” and refers generally to animals raised primarily for food. For example, such animals include, but are not limited to, cattle (bovine), sheep (ovine), pigs (porcine or swine), poultry (avian), and the like. As used herein, the term “cow” or “cattle” is used generally to refer to an animal of bovine origin of any age. Interchangeable terms include “bovine”, “calf”, “steer”, “bull”, “heifer”, “cow” and the like. As used herein, the term “pig” is used generally to refer to an animal of porcine origin of any age. Interchangeable terms include “piglet”, “sow” and the like.

By the term “complementarity” or “complementary” is meant, for the purposes of the specification or claims, a sufficient number in the oligonucleotide of complementary base pairs in its sequence to interact specifically (hybridize) with the target nucleic acid sequence of the ob gene polymorphism to be amplified or detected. As known to those skilled in the art, a very high degree of complementarity is needed for specificity and sensitivity involving hybridization, although it need not be 100%. Thus, for example, an oligonucleotide that is identical in nucleotide sequence to an oligonucleotide disclosed herein, except for one base change or substitution, may function equivalently to the disclosed oligonucleotides. A “complementary DNA” or “cDNA” gene includes recombinant genes synthesized by reverse transcription of messenger RNA (“mRNA”).

A “cyclic polymerase-mediated reaction” refers to a biochemical reaction in which a template molecule or a population of template molecules is periodically and repeatedly copied to create a complementary template molecule or complementary template molecules, thereby increasing the number of the template molecules over time.

“Denaturation” of a template molecule refers to the unfolding or other alteration of the structure of a template so as to make the template accessible to duplication. In the case of DNA, “denaturation” refers to the separation of the two complementary strands of the double helix, thereby creating two complementary, single stranded template molecules. “Denaturation” can be accomplished in any of a variety of ways, including by heat or by treatment of the DNA with a base or other denaturant.

A “detectable amount of product” refers to an amount of amplified nucleic acid that can be detected using standard laboratory tools. A “detectable marker” refers to a nucleotide analog that allows detection using visual or other means. For example, fluorescently labeled nucleotides can be incorporated into a nucleic acid during one or more steps of a cyclic polymerase-mediated reaction, thereby allowing the detection of the product of the reaction using, e.g. fluorescence microscopy or other fluorescence-detection instrumentation.

By the term “detectable moiety” is meant, for the purposes of the specification or claims, a label molecule (isotopic or non-isotopic) which is incorporated indirectly or directly into an oligonucleotide, wherein the label molecule facilitates the detection of the oligonucleotide in which it is incorporated, for example when the oligonucleotide is hybridized to amplified ob gene polymorphisms sequences. Thus, “detectable moiety” is used synonymously with “label molecule”. Synthesis of oligonucleotides can be accomplished by any one of several methods known to those skilled in the art. Label molecules, known to those skilled in the art as being useful for detection, include chemiluminescent or fluorescent molecules. Various fluorescent molecules are known in the art which are suitable for use to label a nucleic acid for the method of the present invention. The protocol for such incorporation may vary depending upon the fluorescent molecule used. Such protocols are known in the art for the respective fluorescent molecule.

By “detectably labeled” is meant that a fragment or an oligonucleotide contains a nucleotide that is radioactive, or that is substituted with a fluorophore, or that is substituted with some other molecular species that elicits a physical or chemical response that can be observed or detected by the naked eye or by means of instrumentation such as, without limitation, scintillation counters, calorimeters, UV spectrophotometers and the like. As used herein, a “label” or “tag” refers to a molecule that, when appended by, for example, without limitation, covalent bonding or hybridization, to another molecule, for example, also without limitation, a polynucleotide or polynucleotide fragment, provides or enhances a means of detecting the other molecule. A fluorescence or fluorescent label or tag emits detectable light at a particular wavelength when excited at a different wavelength. A radiolabel or radioactive tag emits radioactive particles detectable with an instrument such as, without limitation, a scintillation counter. Other signal generation detection methods include: chemiluminescence, electrochemiluminescence, raman, calorimetric, hybridization protection assay, and mass spectrometry

“DNA amplification” as used herein refers to any process that increases the number of copies of a specific DNA sequence by enzymatically amplifying the nucleic acid sequence. A variety of processes are known. One of the most commonly used is the polymerase chain reaction (PCR), which is defined and described in later sections below. The PCR process of Mullis is described in U.S. Pat. Nos. 4,683,195 and 4,683,202. PCR involves the use of a thermostable DNA polymerase, known sequences as primers, and heating cycles, which separate the replicating deoxyribonucleic acid (DNA), strands and exponentially amplify a gene of interest. Any type of PCR, such as quantitative PCR, RT-PCR, hot start PCR, LAPCR, multiplex PCR, touchdown PCR, etc., may be used. Advantageously, real-time PCR is used. In general, the PCR amplification process involves an enzymatic chain reaction for preparing exponential quantities of a specific nucleic acid sequence. It requires a small amount of a sequence to initiate the chain reaction and oligonucleotide primers that will hybridize to the sequence. In PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed.

“DNA” refers to the polymeric form of deoxyribonucleotides (adenine, guanine, thymine, or cytosine) in either single stranded form, or as a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

By the terms “enzymatically amplify” or “amplify” is meant, for the purposes of the specification or claims, DNA amplification, i.e., a process by which nucleic acid sequences are amplified in number. There are several means for enzymatically amplifying nucleic acid sequences. Currently the most commonly used method is the polymerase chain reaction (PCR). Other amplification methods include LCR (ligase chain reaction) which utilizes DNA ligase, and a probe consisting of two halves of a DNA segment that is complementary to the sequence of the DNA to be amplified, enzyme QB replicase and a ribonucleic acid (RNA) sequence template attached to a probe complementary to the DNA to be copied which is used to make a DNA template for exponential production of complementary RNA; strand displacement amplification (SDA); QB replicase amplification (QBRA); self-sustained replication (3 SR); and NASBA (nucleic acid sequence-based amplification), which can be performed on RNA or DNA as the nucleic acid sequence to be amplified.

A “fragment” of a molecule such as a protein or nucleic acid is meant to refer to any portion of the amino acid or nucleotide genetic sequence.

As used herein, the term “genome” refers to all the genetic material in the chromosomes of a particular organism. Its size is generally given as its total number of base pairs. Within the genome, the term “gene” refers to an ordered sequence of nucleotides located in a particular position on a particular chromosome that encodes a specific functional product (e.g., a protein or RNA molecule). For example, it is known that the protein leptin is encoded by the ob (obese) gene and appears to be involved in the regulation of appetite, basal metabolism and fat deposition. In general, an animal's genetic characteristics, as defined by the nucleotide sequence of its genome, are known as its “genotype,” while the animal's physical traits are described as its “phenotype.”

By “heterozygous” or “heterozygous polymorphism” is meant that the two alleles of a diploid cell or organism at a given locus are different, that is, that they have a different nucleotide exchanged for the same nucleotide at the same place in their sequences.

By “homozygous” or “homozygous polymorphism” is meant that the two alleles of a diploid cell or organism at a given locus are identical, that is, that they have the same nucleotide for nucleotide exchange at the same place in their sequences.

By “hybridization” or “hybridizing,” as used herein, is meant the formation of A-T and C-G base pairs between the nucleotide sequence of a fragment of a segment of a polynucleotide and a complementary nucleotide sequence of an oligonucleotide. By complementary is meant that at the locus of each A, C, G or T (or U in a ribonucleotide) in the fragment sequence, the oligonucleotide sequenced has a T, G, C or A, respectively. The hybridized fragment/oligonucleotide is called a “duplex.”

A “hybridization complex”, such as in a sandwich assay, means a complex of nucleic acid molecules including at least the target nucleic acid and a sensor probe. It may also include an anchor probe.

By “immobilized on a solid support” is meant that a fragment, primer or oligonucleotide is attached to a substance at a particular location in such a manner that the system containing the immobilized fragment, primer or oligonucleotide may be subjected to washing or other physical or chemical manipulation without being dislodged from that location. A number of solid supports and means of immobilizing nucleotide-containing molecules to them are known in the art; any of these supports and means may be used in the methods of this invention.

As used herein, the term “increased weight gain” means a biologically significant increase in weight gain above the mean of a given population.

As used herein, the term “locus” or “loci” refers to the site of a gene on a chromosome. A single allele from each locus is inherited from each parent. Each animal's particular combination of alleles is referred to as its “genotype”. Where both alleles are identical, the individual is said to be homozygous for the trait controlled by that pair of alleles; where the alleles are different, the individual is said to be heterozygous for the trait.

A “melting temperature” is meant the temperature at which hybridized duplexes dehybridize and return to their single-stranded state. Likewise, hybridization will not occur in the first place between two oligonucleotides, or, herein, an oligonucleotide and a fragment, at temperatures above the melting temperature of the resulting duplex. It is presently advantageous that the difference in melting point temperatures of oligonucleotide-fragment duplexes of this invention be from about 1° C. to about 110° C. so as to be readily detectable.

As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but advantageously is double-stranded DNA. An “isolated” nucleic acid molecule is one that is separated from other nucleic acid molecules that are present in the natural source of the nucleic acid. A “nucleoside” refers to a base linked to a sugar. The base may be adenine (A), guanine (G) (or its substitute, inosine (I)), cytosine (C), or thymine (T) (or its substitute, uracil (U)). The sugar may be ribose (the sugar of a natural nucleotide in RNA) or 2-deoxyribose (the sugar of a natural nucleotide in DNA). A “nucleotide” refers to a nucleoside linked to a single phosphate group.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides may be chemically synthesized and may be used as primers or probes. Oligonucleotide means any nucleotide of more than 3 bases in length used to facilitate detection or identification of a target nucleic acid, including probes and primers.

“Polymerase chain reaction” or “PCR” refers to a thermocyclic, polymerase-mediated, DNA amplification reaction. A PCR typically includes template molecules, oligonucleotide primers complementary to each strand of the template molecules, a thermostable DNA polymerase, and deoxyribonucleotides, and involves three distinct processes that are multiply repeated to effect the amplification of the original nucleic acid. The three processes (denaturation, hybridization, and primer extension) are often performed at distinct temperatures, and in distinct temporal steps. In many embodiments, however, the hybridization and primer extension processes can be performed concurrently. The nucleotide sample to be analyzed may be PCR amplification products provided using the rapid cycling techniques described in U.S. Pat. Nos. 6,569,672; 6,569,627; 6,562,298; 6,556,940; 6,569,672; 6,569,627; 6,562,298; 6,556,940; 6,489,112; 6,482,615; 6,472,156; 6,413,766; 6,387,621; 6,300,124; 6,270,723; 6,245,514; 6,232,079; 6,228,634; 6,218,193; 6,210,882; 6,197,520; 6,174,670; 6,132,996; 6,126,899; 6,124,138; 6,074,868; 6,036,923; 5,985,651; 5,958,763; 5,942,432; 5,935,522; 5,897,842; 5,882,918; 5,840,573; 5,795,784; 5,795,547; 5,785,926; 5,783,439; 5,736,106; 5,720,923; 5,720,406; 5,675,700; 5,616,301; 5,576,218 and 5,455,175, the disclosures of which are incorporated by reference in their entireties. Other methods of amplification include, without limitation, NASBR, SDA, 3SR, TSA and rolling circle replication. It is understood that, in any method for producing a polynucleotide containing given modified nucleotides, one or several polymerases or amplification methods may be used. The selection of optimal polymerization conditions depends on the application.

A “polymerase” is an enzyme that catalyzes the sequential addition of monomeric units to a polymeric chain, or links two or more monomeric units to initiate a polymeric chain. In advantageous embodiments of this invention, the “polymerase” will work by adding monomeric units whose identity is determined by and which is complementary to a template molecule of a specific sequence. For example, DNA polymerases such as DNA pol 1 and Taq polymerase add deoxyribonucleotides to the 3′ end of a polynucleotide chain in a template-dependent manner, thereby synthesizing a nucleic acid that is complementary to the template molecule. Polymerases may be used either to extend a primer once or repetitively or to amplify a polynucleotide by repetitive priming of two complementary strands using two primers.

A “polynucleotide” refers to a linear chain of nucleotides connected by a phosphodiester linkage between the 3′-hydroxyl group of one nucleoside and the 5′-hydroxyl group of a second nucleoside which in turn is linked through its 3′-hydroxyl group to the 5′-hydroxyl group of a third nucleoside and so on to form a polymer comprised of nucleosides liked by a phosphodiester backbone. A “modified polynucleotide” refers to a polynucleotide in which one or more natural nucleotides have been partially or substantially replaced with modified nucleotides.

A “primer” is an oligonucleotide, the sequence of at least a portion of which is complementary to a segment of a template DNA which to be amplified or replicated. Typically primers are used in performing the polymerase chain reaction (PCR). A primer hybridizes with (or “anneals” to) the template DNA and is used by the polymerase enzyme as the starting point for the replication/amplification process. By “complementary” is meant that the nucleotide sequence of a primer is such that the primer can form a stable hydrogen bond complex with the template; i.e., the primer can hybridize or anneal to the template by virtue of the formation of base-pairs over a length of at least ten consecutive base pairs.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

“Probes” refer to oligonucleotide nucleic acid sequences of variable length, used in the detection of identical, similar, or complementary nucleic acid sequences by hybridization. An oligonucleotide sequence used as a detection probe may be labeled with a detectable moiety. Various labeling moieties are known in the art. Said moiety may, for example, either be a radioactive compound, a detectable enzyme (e.g., horse radish peroxidase (HRP)) or any other moiety capable of generating a detectable signal such as a calorimetric, fluorescent, chemiluminescent or electrochemiluminescent signal. The detectable moiety may be detected using known methods.

As used herein, the term “protein” refers to a large molecule composed of one or more chains of amino acids in a specific order. The order is determined by the base sequence of nucleotides in the gene coding for the protein. Proteins are required for the structure, function, and regulation of the body's cells, tissues, and organs. Each protein has a unique function.

As used herein, the terms “traits,” or “physical characteristics” refer to advantageous properties of the animal resulting from genetics. Quality traits include, but are not limited to, the traits related to an animl's carcass quality, quantifiable trait such as the animal's genetic ability to metabolize energy, produce milk, put on intramuscular fat, lay eggs, produce offspring, produce particular proteins in meat or milk, or retain protein in milk. Physical characteristics include, but are not limited to, marbled or lean meats or meat tenderness. The terms are used interchangeably. Performance traits include, but are not limited to, live weight, dry material intake, residual feed intake, feeding duration, feedbunk visits, metabolic midpoint weight and the like.

A “restriction enzyme” refers to an endonuclease (an enzyme that cleaves phosphodiester bonds within a polynucleotide chain) that cleaves DNA in response to a recognition site on the DNA. The recognition site (restriction site) consists of a specific sequence of nucleotides typically about 4-8 nucleotides long.

A “single nucleotide polymorphism” or “SNP” refers to polynucleotide that differs from another polynucleotide by a single nucleotide exchange. For example, without limitation, exchanging one A for one C, G, or T in the entire sequence of polynucleotide constitutes a SNP. Of course, it is possible to have more than one SNP in a particular polynucleotide. For example, at one locus in a polynucleotide, a C may be exchanged for a T, at another locus a G may be exchanged for an A, and so on. When referring to SNPs, the polynucleotide is most often DNA.

As used herein, a “template” refers to a target polynucleotide strand, for example, without limitation, an unmodified naturally-occurring DNA strand, which a polymerase uses as a means of recognizing which nucleotide it should next incorporate into a growing strand to polymerize the complement of the naturally-occurring strand. Such DNA strand may be single-stranded or it may be part of a double-stranded DNA template. In applications of the present invention requiring repeated cycles of polymerization, e.g., the polymerase chain reaction (PCR), the template strand itself may become modified by incorporation of modified nucleotides, yet still serve as a template for a polymerase to synthesize additional polynucleotides.

A “thermocyclic reaction” is a multi-step reaction wherein at least two steps are accomplished by changing the temperature of the reaction.

A “thermostable polymerase” refers to a DNA or RNA polymerase enzyme that can withstand extremely high temperatures, such as those approaching 100° C. Often, thermostable polymerases are derived from organisms that live in extreme temperatures, such as Thermus aquaticus. Examples of thermostable polymerases include Taq, Tth, Pfu, Vent, deep vent, UlTma, and variations and derivatives thereof.

A “variance” is a difference in the nucleotide sequence among related polynucleotides. The difference may be the deletion of one or more nucleotides from the sequence of one polynucleotide compared to the sequence of a related polynucleotide, the addition of one or more nucleotides or the substitution of one nucleotide for another. The terms “mutation,” “polymorphism” and “variance” are used interchangeably herein. As used herein, the term “variance” in the singular is to be construed to include multiple variances; i.e., two or more nucleotide additions, deletions and/or substitutions in the same polynucleotide. A “point mutation” refers to a single substitution of one nucleotide for another.

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein.

Further definitions are provided in context below. Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein.

The present invention encompasses methods for the identification and selection of animals based on the presence of SNPs in the ob (obese) gene—a gene that encodes the protein leptin. Leptin is a 16-kDa adipocyte-specific polypeptide involved in the regulation of appetite, basal metabolism, fat deposition and milk production. The ob gene has been mapped to specific chromosomes in several different animals, allowing the gene to be sequenced in several different species. It has been found that there is significant conservation of ob DNAs and leptin polypeptides between species. SNPs having the same or similar phenotypic effects to those of the present invention may occur in many different animal species. The methods of the present invention can be used to determine whether an individual animal from a species of interest possesses the SNPs described herein. In advantageous embodiments, the ob gene of a bovine animal is screened for the presence of the SNPs of the present invention.

In one aspect, the present invention relates to the identification of single nucleotide polymorphisms (SNPs) in the leptin promoter, and to methods for the identification of animals carrying specific alleles of these SNPs that are associated with circulating leptin levels, feed intake, growth rate, body weight, carcass merit and composition, and milk yield. In a further aspect, the present invention relates the association of a previously reported SNP in exon 2 of the leptin gene, with circulating leptin levels, feed intake, growth rate, body weight, carcass merit and composition, milk yield and the like. The present invention also provides oligonucleotides that can be used as primers to amplify specific nucleic acid sequences of the ob gene, and oligonucleotides that can be used as probes in the detection of nucleic acid sequences of the ob gene. In another aspect, the invention relates the association of a previously reported SNP in exon 8 of the bovine growth hormone receptor (bGHr) gene, with feed intake, dry matter intake, daily feed intake to milk ratio, dry matter over milk ratio and cumulative effective energy balance (CEEB). The invention also encompasses combinations of these SNPs that together are associated with carcass and performance traits of beef and/or dairy cattle.

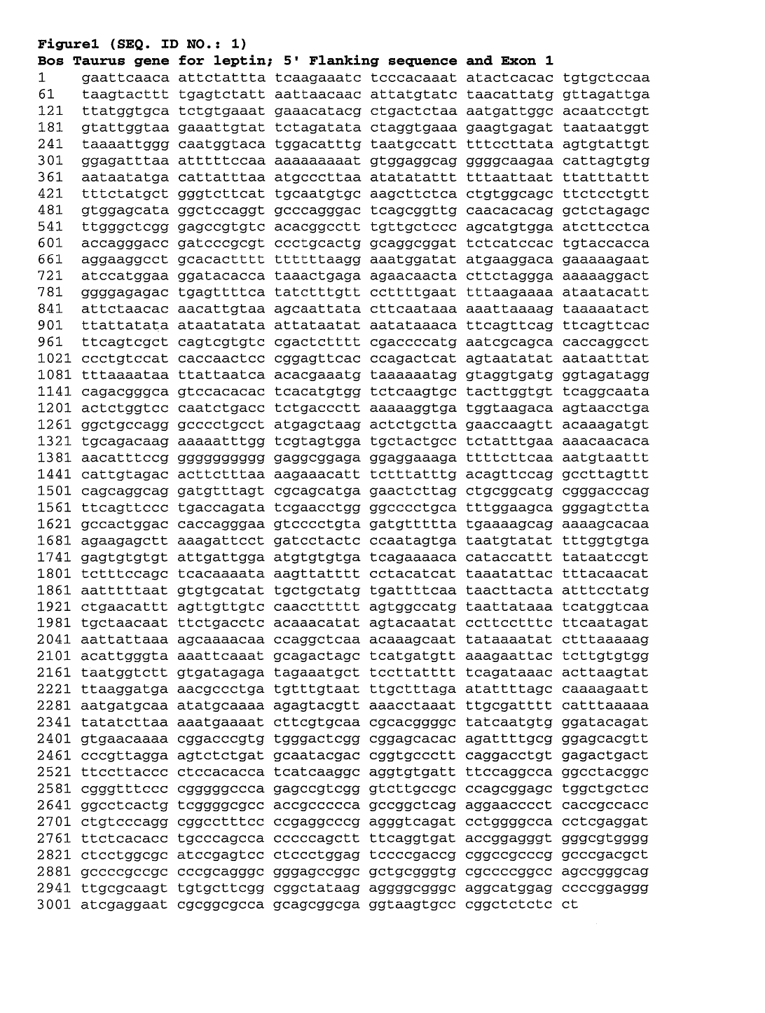

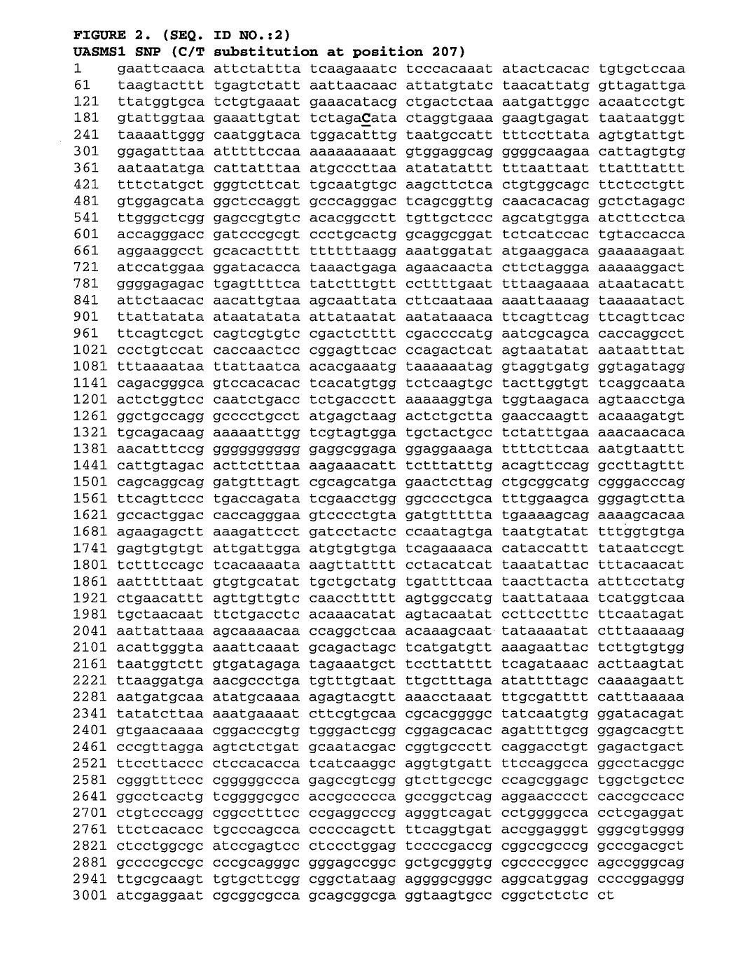

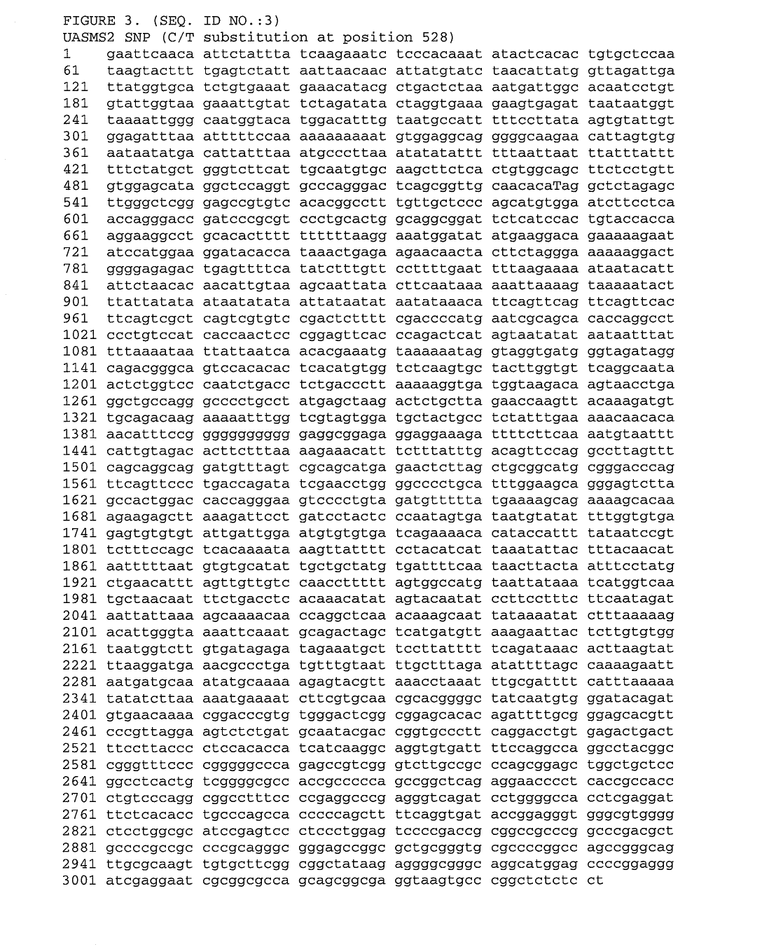

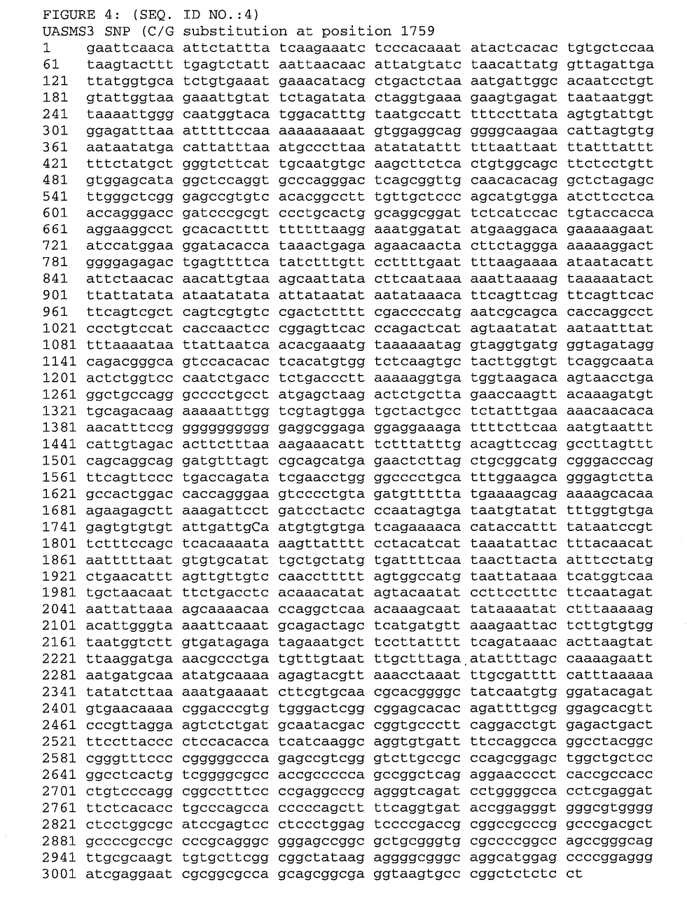

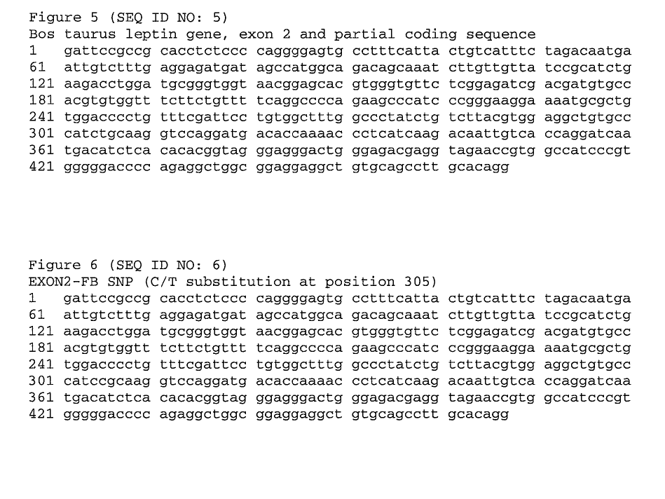

FIG. 1 illustrates the nucleotide sequence for the 5′ flanking promoter region and exon 1 of the “wild type” bovine ob gene. This “wild type” sequence has GenBank accession number AB070368, and is designated herein as SEQ ID NO: 1.

In the present invention it has surprisingly been shown that three previously unknown SNPs (namely UASMS1, UASMS2 and UASMS3) located in the promoter region of the ob gene, and one previously known SNP in exon 2 of the gene are associated with certain economically valuable traits in animals, in particular in bovine livestock.

The SNP termed UASMSI constitutes a cytosine (C) to thymine (T) substitution (C/T) at position 207 of the bovine leptin gene promoter. The SNP termed UASMS2, constitutes a cytosine (C) to thymine (T) substitution (C/T substitution) at position 528 of bovine leptin gene promoter. The SNP termed UASMS3 constitutes a cytosine (C) to guanine (G) substitution (C/G substitution) at position 1759 of the bovine leptin gene promoter. The nucleotide numbering system used herein for the identification of the leptin promoter SNPs UASMS1, UASMS2 and UASMS3 is that used for the “wild type” bovine leptin promoter sequence SEQ ID NO: 1.

The UASMS1, UASMS2 and UASMS3 polymorphisms are located in the 5′ regulatory sequence of the leptin gene, not the coding region of the gene, and thus do not result in any amino acid substitution in the leptin gene product.

The SNP termed EXON2-FB described herein was identified previously by Buchanan et al. (2002), and constitutes a cytosine (C) to thymine (T) missense mutation at position 305 in exon 2 of the coding region of the “wild type” bovine leptin gene (GenBank accession No. AY138588, and SEQ ID NO: 5). The nucleotide numbering system used herein for the identification of the EXON2-FB SNP is that used for the “wild type” bovine leptin exon 2 sequence SEQ ID NO: 5.

The SNP termed E2JW described herein was identified previously by Lagoniro et al. (2002), and constitutes a cytosine (C) to thymine (T) missense mutation at position 1759 in exon 2 of the coding region of the “wild type” bovine leptin gene (GenBank accession No. AY138588, and SEQ ID NO: 5). The nucleotide numbering system used herein for the identification of the EXON2-FB SNP is that used for the “wild type” bovine leptin exon 2 sequence SEQ ID NO: 5.

In order to determine the genotype of a given animal according to the methods of the present invention, it is necessary to obtain a sample of genomic DNA from that animal. Typically, that sample of genomic DNA will be obtained from a sample of tissue or cells taken from that animal.

A tissue or cell sample may be taken from an animal at any time in the lifetime of an animal but before the carcass identity is lost. The tissue sample can comprise hair (including roots), hide, bone, buccal swabs, blood, saliva, milk, semen, embryos, muscle or any internal organs. In the method of the present invention, the source of the tissue sample, and thus also the source of the test nucleic acid sample, is not critical. For example, the test nucleic acid can be obtained from cells within a body fluid of the animal, or from cells constituting a body tissue of the animal. The particular body fluid from which cells are obtained is also not critical to the present invention. For example, the body fluid may be selected from the group consisting of blood, ascites, pleural fluid and spinal fluid. Furthermore, the particular body tissue from which cells are obtained is also not critical to the present invention. For example, the body tissue may be selected from the group consisting of skin, endometrial, uterine and cervical tissue. Both normal and tumor tissues can be used.

Typically, the tissue sample is marked with an identifying number or other indicia that relates the sample to the individual animal from which the sample was taken. The identity of the sample advantageously remains constant throughout the methods of the invention thereby guaranteeing the integrity and continuity of the sample during extraction and analysis. Alternatively, the indicia may be changed in a regular fashion that ensures that the data, and any other associated data, can be related back to the animal from which the data was obtained.

The amount/size of sample required is known to those skilled in the art. Ideally, the size/volume of the tissue sample retrieved should be as consistent as possible within the type of sample and the species of animal. For example, for cattle, non-limiting examples of sample sizes/methods include non-fatty meat: 0.0002 to 0.0010 g; hide: 0.0004 to 0.0010 g; hair roots: greater than five and less than twenty; buccal swabs: 15 to 20 seconds of rubbing with modest pressure in the area between outer lip and gum using one Cytosoft® cytology brush; bone: 0.0020 to 0.0040 g; and blood: 30 to 70 μL.

Generally, the tissue sample is placed in a container that is labeled using a numbering system bearing a code corresponding to the animal, for example, to the animal's ear tag. Accordingly, the genotype of a particular animal is easily traceable at all times.

In one embodiment of the invention, a sampling device and/or container may be supplied to the farmer, a slaughterhouse or retailer. The sampling device advantageously takes a consistent and reproducible sample from individual animals while simultaneously avoiding any cross-contamination of tissue. Accordingly, the size and volume of sample tissues derived from individual animals would be consistent.

According to the present invention, a sample of genomic DNA is obtained from the tissue sample of the livestock animal of interest. Whatever source of cells or tissue is used, a sufficient amount of cells must be obtained to provide a sufficient amount of DNA for analysis. This amount will be known or readily determinable by those skilled in the art.

DNA is isolated from the tissue/cells by techniques known to those skilled in the art (see, e.g., U.S. Pat. Nos. 6,548,256 and 5,989,431, Hirota et al., Jinrui Idengaku Zasshi. 1989 September; 34(3):217-23 and John et al., Nucleic Acids Res. 1991 Jan. 25; 19(2):408; the disclosures of which are incorporated by reference in their entireties). For example, high molecular weight DNA may be purified from cells or tissue using proteinase K extraction and ethanol precipitation. DNA may be extracted from an animal specimen using any other suitable methods known in the art.

It is an object of the present invention to determine the genotype of a given animal of interest, in order to identify animals carrying specific alleles of the SNPs of the invention that are associated with circulating leptin levels, feed intake, growth rate, body weight, carcass merit and composition, and milk yield.

There are many methods known in the art for determining the genotype of an animal and for identifying whether a given DNA sample contains a particular SNP. Any method for determining genotype can be used for determining the ob genotype in the present invention. Such methods include, but are not limited to, amplifier sequencing, DNA sequencing, fluorescence spectroscopy, fluorescence resonance energy transfer (or “FRET”)-based hybridization analysis, high throughput screening, mass spectroscopy, nucleic acid hybridization, polymerase chain reaction (PCR), RFLP analysis and size chromatography (e.g., capillary or gel chromatography), all of which are well known to one of skill in the art. In particular, methods for determining nucleotide polymorphisms, particularly single nucleotide polymorphisms, are described in U.S. Pat. Nos. 6,514,700; 6,503,710; 6,468,742; 6,448,407; 6,410,231; 6,383,756; 6,358,679; 6,322,980; 6,316,230; and 6,287,766 and reviewed by Chen and Sullivan, Pharmacogenomics J 2003; 3(2):77-96, the disclosures of which are incorporated by reference in their entireties.

In one embodiment, the presence or absence of the SNPs of the present invention is determined by sequencing the region of the genomic DNA sample that spans the polymorphic locus. Many methods of sequencing genomic DNA are known in the art, and any such method can be used, see for example Sambrook et al., Molecular Cloning; A Laboratory Manual 2d ed. (1989). For example, as described below, a DNA fragment spanning the location of the SNP of interest can amplified using the polymerase chain reaction or some other cyclic polymerase mediated amplification reaction. The amplified region of DNA can then be sequenced using any method known in the art. Advantageously, the nucleic acid sequencing is by automated methods (reviewed by Meldrum, Genome Res. 2000 September; 10(9):1288-303, the disclosure of which is incorporated by reference in its entirety), for example using a Beckman CEQ 8000 Genetic Analysis System (Beckman Coulter Instruments, Inc.). Methods for sequencing nucleic acids include, but are not limited to, automated fluorescent DNA sequencing (see, e.g., Watts & MacBeath, Methods Mol Biol. 2001; 167:153-70 and MacBeath et al., Methods Mol Biol. 2001; 167:119-52), capillary electrophoresis (see, e.g., Bosserhoff et al., Comb Chem High Throughput Screen. 2000 December; 3(6):455-66), DNA sequencing chips (see, e.g., Jain, Pharmacogenomics. 2000 August; 1(3):289-307), mass spectrometry (see, e.g., Yates, Trends Genet. 2000 January; 16(1):5-8), pyrosequencing (see, e.g., Ronaghi, Genome Res. 2001 January; 11(1):3-11), and ultrathin-layer gel electrophoresis (see, e.g., Guttman & Ronai, Electrophoresis. 2000 December; 21(18):3952-64), the disclosures of which are hereby incorporated by reference in their entireties. The sequencing can also be done by any commercial company. Examples of such companies include, but are not limited to, the University of Georgia Molecular Genetics Instrumentation Facility (Athens, Ga.) or SeqWright DNA Technologies Services (Houston, Tex.).

In certain embodiments of the present invention, the detection of a given SNP can be performed using cyclic polymerase-mediated amplification methods. Any one of the methods known in the art for amplification of DNA may be used, such as for example, the polymerase chain reaction (PCR), the ligase chain reaction (LCR) (Barany, F., Proc. Natl. Acad. Sci. (U.S.A.) 88:189-193 (1991)), the strand displacement assay (SDA), or the oligonucleotide ligation assay (“OLA”) (Landegren, U. et al., Science 241:1077-1080 (1988)). Nickerson, D. A. et al. have described a nucleic acid detection assay that combines attributes of PCR and OLA (Nickerson, D. A. et al, Proc. Natl. Acad. Sci. (U.S.A.) 87:8923-8927 (1990)). Other known nucleic acid amplification procedures, such as transcription-based amplification systems (Malek, L. T. et al, U.S. Pat. No. 5,130,238; Davey, C. et al., European Patent Application 329,822; Schuster et al., U.S. Pat. No. 5,169,766; Miller, H. I. et al., PCT Application WO89/06700; Kwoh, D. et al., Proc. Natl. Acad. Sci. (U.S.A.) 86:1173 (1989); Gingeras, T. R. et al., PCT Application WO88/10315)), or isothermal amplification methods (Walker, G. T. et al., Proc. Natl. Acad. Sci. (U.S.A.) 89:392-396 (1992)) may also be used.

The most advantageous method of amplifying DNA fragments containing the SNPs of the invention employs PCR (see e.g., U.S. Pat. Nos. 4,965,188; 5,066,584; 5,338,671; 5,348,853; 5,364,790; 5,374,553; 5,403,707; 5,405,774; 5,418,149; 5,451,512; 5,470,724; 5,487,993; 5,523,225; 5,527,510; 5,567,583; 5,567,809; 5,587,287; 5,597,910; 5,602,011; 5,622,820; 5,658,764; 5,674,679; 5,674,738; 5,681,741; 5,702,901; 5,710,381; 5,733,751; 5,741,640; 5,741,676; 5,753,467; 5,756,285; 5,776,686; 5,811,295; 5,817,797; 5,827,657; 5,869,249; 5,935,522; 6,001,645; 6,015,534; 6,015,666; 6,033,854; 6,043,028; 6,077,664; 6,090,553; 6,168,918; 6,174,668; 6,174,670; 6,200,747; 6,225,093; 6,232,079; 6,261,431; 6,287,769; 6,306,593; 6,440,668; 6,468,743; 6,485,909; 6,511,805; 6,544,782; 6,566,067; 6,569,627; 6,613,560; 6,613,560 and 6,632,645; the disclosures of which are incorporated by reference in their entireties), using primer pairs that are capable of hybridizing to the proximal sequences that define or flank a polymorphic site in its double-stranded form.

To perform a cyclic polymerase mediated amplification reaction according to the present invention, the primers are hybridized or annealed to opposite strands of the target DNA, the temperature is then raised to permit the thermostable DNA polymerase to extend the primers and thus replicate the specific segment of DNA spanning the region between the two primers. Then the reaction is thermocycled so that at each cycle the amount of DNA representing the sequences between the two primers is doubled, and specific amplification of the ob gene DNA sequences, if present, results.

Any of a variety of polymerases can be used in the present invention. For thermocyclic reactions, the polymerases are thermostable polymerases such as Taq, KlenTaq, Stoffel Fragment, Deep Vent, Tth, Pfu, Vent, and UlTma, each of which are readily available from commercial sources. For non-thermocyclic reactions, and in certain thermocyclic reactions, the polymerase will often be one of many polymerases commonly used in the field, and commercially available, such as DNA pol 1, Klenow fragment, T7 DNA polymerase, and T4 DNA polymerase. Guidance for the use of such polymerases can readily be found in product literature and in general molecular biology guides.

Typically, the annealing of the primers to the target DNA sequence is carried out for about 2 minutes at about 37-55° C., extension of the primer sequence by the polymerase enzyme (such as Taq polymerase) in the presence of nucleoside triphosphates is carried out for about 3 minutes at about 70-75° C., and the denaturing step to release the extended primer is carried out for about 1 minute at about 90-95° C. However, these parameters can be varied, and one of skill in the art would readily know how to adjust the temperature and time parameters of the reaction to achieve the desired results. For example, cycles may be as short as 10, 8, 6, 5, 4.5, 4, 2, 1, 0.5 minutes or less.

Also, “two temperature” techniques can be used where the annealing and extension steps may both be carried out at the same temperature, typically between about 60-65° C., thus reducing the length of each amplification cycle and resulting in a shorter assay time.

Typically, the reactions described herein are repeated until a detectable amount of product is generated. Often, such detectable amounts of product are between about 10 ng and about 100 ng, although larger quantities, e.g. 200 ng, 500 ng, 1 mg or more can also, of course, be detected. In terms of concentration, the amount of detectable product can be from about 0.01 pmol, 0.1 pmol, 1 pmol, 10 pmol, or more. Thus, the number of cycles of the reaction that are performed can be varied, the more cycles are performed, the more amplified product is produced. In certain embodiments, the reaction comprises 2, 5, 10, 15, 20, 30, 40, 50, or more cycles.

For example, the PCR reaction may be carried out using about 25-50 μl samples containing about 0.01 to 1.0 ng of template amplification sequence, about 10 to 100 pmol of each generic primer, about 1.5 units of Taq DNA polymerase (Promega Corp.), about 0.2 mM dDATP, about 0.2 mM dCTP, about 0.2 mM dGTP, about 0.2 mM dTTP, about 15 mM MgCl₂, about 10 mM Tris-HCl (pH 9.0), about 50 mM KCl, about 1 μg/ml gelatin, and about 10 μl/ml Triton X-100 (Saiki, 1988).

Those of skill in the art are aware of the variety of nucleotides available for use in the cyclic polymerase mediated reactions. Typically, the nucleotides will consist at least in part of deoxynucleotide triphosphates (dNTPs), which are readily commercially available. Parameters for optimal use of dNTPs are also known to those of skill, and are described in the literature. In addition, a large number of nucleotide derivatives are known to those of skill and can be used in the present reaction. Such derivatives include fluorescently labeled nucleotides, allowing the detection of the product including such labeled nucleotides, as described below. Also included in this group are nucleotides that allow the sequencing of nucleic acids including such nucleotides, such as chain-terminating nucleotides, dideoxynucleotides and boronated nuclease-resistant nucleotides. Commercial kits containing the reagents most typically used for these methods of DNA sequencing are available and widely used. Other nucleotide analogs include nucleotides with bromo-, iodo-, or other modifying groups, which affect numerous properties of resulting nucleic acids including their antigenicity, their replicatability, their melting temperatures, their binding properties, etc. In addition, certain nucleotides include reactive side groups, such as sulfhydryl groups, amino groups, N-hydroxysuccinimidyl groups, that allow the further modification of nucleic acids comprising them.

The present invention provides oligonucleotides that can be used as primers to amplify specific nucleic acid sequences of the ob gene in cyclic polymerase-mediated amplification reactions, such as PCR reactions. These primers are useful in detecting the UASMS1, UASMS2 or UASMS3 SNPs in the leptin promoter, and the EXON2-FB SNP in exon 2 of the leptin gene. In certain embodiments, these primers consist of oligonucleotide fragments. Such fragments should be of sufficient length to enable specific annealing or hybridization to the nucleic acid sample. The sequences typically will be about 8 to about 44 nucleotides in length, but may be longer. Longer sequences, e.g., from about 14 to about 50, are advantageous for certain embodiments.

In embodiments where it is desired to amplify a fragment of DNA comprising the UASMS1, UASMS2 or UASMS3 SNPs, primers having contiguous stretches of about 8, 9, 10, 11, 12, 13, 14, 15, 16, 17, 18, 19, 20, 21, 22, 23, or 24 nucleotides from SEQ ID NO: 1 (the leptin promoter sequence) are contemplated. In embodiments where it is desired to amplify a fragment of DNA comprising the EXON2-FB SNP, primers having contiguous stretches of about 8, 9, 10, 11, 12, 13, 14, 15, 16, 17, 18, 19, 20, 21, 22, 23, or 24 nucleotides from SEQ ID NO: 5 (exon 2 of the leptin gene) are contemplated.

Although various different lengths of primers can be used, and the exact location of the stretch of contiguous nucleotides in leptin gene used to make the primer can vary, it is important that the sequences to which the forward and reverse primers anneal are located on either side of the particular nucleotide position that is substituted in the SNP to be amplified. For example, when designing primers for amplification of the UASMS1 polymorphism, one primer must be located upstream of (not overlapping with) nucleotide position 207 of the leptin promoter (SEQ ID NO: 1, or 2), and the other primer must be located downstream of (not overlapping with) nucleotide position 207 of the leptin promoter (SEQ ID NO: 1, or 2). When designing primers for amplification of the UASMS2 polymorphism, one primer must be located upstream of (not overlapping with) nucleotide position 528 of the leptin promoter (SEQ ID NO: 1, or 3), and the other primer must be located downstream of (not overlapping with) nucleotide position 528 of the leptin promoter (SEQ ID NO: 1, or 3). Similarly, when designing primers for amplification of the UASMS3 polymorphism one primer must be located upstream of (not overlapping with) nucleotide position 1759 of the leptin promoter (SEQ ID NO: 1, or 4), and the other primer must be located downstream of (not overlapping with) nucleotide position 1759. Finally, when designing primers for amplification of the EXON2-FB polymorphism one primer must be located upstream of (not overlapping with) nucleotide position 305 of exon 2 (SEQ ID NO: 5), and the other primer must be located downstream of (not overlapping with) nucleotide position 305 of exon 2.

In a preferred embodiment, a fragment of DNA spanning and containing the location of the UASMS1 polymorphism is amplified from a nucleic acid sample using a forward primer having the sequence 5′-GGCACAATCCTGTGTATTGGTAAGA-3′ (SEQ ID NO: 7), and a reverse primer having the sequence 5′-GTCCATGTACCATTGCCCAATTT-3′ (SEQ ID NO: 8).

Similarly, in a preferred embodiment, a fragment of DNA spanning the location of the UASMS2 polymorphism is amplified from a nucleic acid sample using a forward primer having the sequence 5′-AGGTGCCCAGGGACTCA-3′(SEQ ID NO: 11), and a reverse primer having the sequence 5′-CAACAAAGGCCGTGTGACA-3′ (SEQ ID NO: 12).

For amplification of a fragment of DNA spanning the location of the UASMS3 polymorphism, it is preferred that a forward primer having the sequence 5′-ATGTATATTTGGTGTGAGAGTGTGTGT-3′ (SEQ ID NO: 15), and a reverse primer having the sequence 5′-AGCTGGAAAGAACGGATTATAAAATGGT-3′ (SEQ ID NO: 16), is used.

Likewise, for amplification of a fragment of DNA spanning the location of the EXON2-FB polymorphism, it is preferred that a forward primer having the sequence 5′-GGCTTTGGCCCTATCTGTCTTAC-3′ (SEQ ID NO: 19), and a reverse primer having the sequence 5′-CTTGATGAGGGTTTTGGTGTCA-3′ (SEQ ID NO: 20), is used.

The above methods employ primers located on either side of, and not overlapping with, the SNP in order to amplify a fragment of DNA that includes the nucleotide position at which the SNP is located. Such methods require additional steps, such as sequencing of the fragment, or hybridization of allele specific probes to the fragment, in order to determine the genotype at the polymorphic site. However, in some embodiments of the present invention, the amplification method is itself a method for determining the genotype of the polymorphic site, as for example, in “allele-specific PCR”. In allele-specific PCR, primer pairs are chosen such that amplification itself is dependent upon the input template nucleic acid containing the polymorphism of interest. In such embodiments, primer pairs are chosen such that at least one primer spans the actual nucleotide position of the SNP and is therefore an allele-specific oligonucleotide primer. Typically, the primers contain a single allele-specific nucleotide at the 3′ terminus preceded by bases that are complementary to the gene of interest. The PCR reaction conditions are adjusted such that amplification by a DNA polymerase proceeds from matched 3′-primer termini, but does not proceed where a mismatch occurs. Allele specific PCR can be performed in the presence of two different allele-specific primers, one specific for each allele, where each primer is labeled with a different dye, for example one allele specific primer may be labeled with a green dye (e.g. fluorescein) and the other allele specific primer labeled with a red dye (e.g. sulforhodamine). Following amplification, the products are analyzed for green and red fluorescence. The aim is for one homozygous genotype to yield green fluorescence only, the other homozygous genotype to give red fluorescence only, and the heterozygous genotype to give mixed red and green fluorescence.

Thus, to perform allele specific PCR to detect the UASMS1 polymorphism, one primer must overlap nucleotide position 207 of SEQ ID NO: 1 or SEQ ID NO: 2 such that nucleotide position 207 is at the 3′ terminus of the primer. Similarly, to perform allele specific PCR to detect the UASMS2 polymorphism, one primer must overlap nucleotide position 528 of SEQ ID NO: 1 or SEQ ID NO: 3 such that nucleotide position 528 is at the 3′ terminus of the primer. To perform allele specific PCR to detect the UASMS3 polymorphism, one primer must overlap nucleotide position 1759 of SEQ ID NO: 1 or SEQ ID NO: 4 such that nucleotide position 1759 is at the 3′ terminus of the primer. Finally, when designing allele specific primers for detection of the EXON2-FB polymorphism, one primer must overlap nucleotide position 305 of SEQ ID NO: 5 or SEQ ID NO: 6 such that nucleotide position 305 is at the 3′ terminus of the primer.

Methods for performing allele specific PCR are well known in the art, and any such methods may be used. For example, suitable methods are taught in Myakishev et al. Genome Research, vol 1, p 163-169 (2001), Alexander et al. Mol Biotechnol. vol 28(3), p 171-174 (2004), and Ruano et al. Nucleic Acids Res. vol 17(20), p 8392 (1989), the contents of which are incorporated by reference. In some embodiments of the present invention, allele-specific primers are chosen so that amplification creates a restriction site, facilitating identification of a polymorphic site. To perform, allele specific PCR the reaction conditions must be carefully adjusted such that the allele specific primer will only bind to one allele and not the alternative allele, for example, in some embodiments the conditions are adjusted so that the primers will only bind where there is a 100% match between the primer sequence and the DNA, and will not bind if there is a single nucleotide mismatch.

In certain embodiments of the present invention, the detection of a given SNP can be performed using oligonucleotide probes that bind or hybridize to the DNA. The present invention provides oligonucleotide probes to detect the UASMS1, UASMS2 or UASMS3 SNPs in the bovine leptin promoter, or the EXON2-FB SNP in exon 2 of the bovine leptin gene.

In certain embodiments, these probes consist of oligonucleotide fragments. Such fragments should be of sufficient length to provide specific hybridization to the nucleic acid sample. The sequences typically will be about 8 to about 50 nucleotides, but may be longer. Nucleic acid probes having contiguous stretches of about 8, 9, 10, 11, 12, 13, 14, 15, 16, 17, 18, 19, 20, 21, 22, 23, or 24 nucleotides from a sequence selected from SEQ ID NO: 1 (wild-type bovine leptin promoter), SEQ ID NO: 2 (bovine leptin promoter with UASMS1 polymorphism), SEQ ID NO: 3 (bovine leptin promoter with UASMS2 polymorphism), SEQ ID NO: 4 (bovine leptin promoter with UASMS3 polymorphism), SEQ ID NO: 5 (wild-type bovine leptin exon 2) or SEQ ID NO: 6 (leptin exon 2 with EXON2-FB polymorphism) are contemplated.

Although various different lengths of probes can be used, and the precise location of the stretch of contiguous nucleotides in the leptin gene from which the probe sequence is derived can vary, the probe sequence must span the particular nucleotide position that is substituted in the particular SNP to be detected. For example, probes designed for detection of the bovine UASMS1 polymorphism must span nucleotide position 207 of the bovine leptin promoter (SEQ ID NO: 2). Probes designed for detection of the bovine UASMS2 polymorphism must span nucleotide position 528 of the bovine leptin promoter (SEQ ID NO: 3). Similarly, probes designed for detection of the bovine UASMS3 polymorphism must span nucleotide position 1759 of the bovine leptin promoter (SEQ ID NO: 4). Finally, probes designed for detection of the bovine exon2-FB polymorphism must span nucleotide position 305 of exon 2 of the bovine leptin gene (SEQ ID NO: 6).

These probes will be useful in a variety of hybridization embodiments, such as Southern blotting, Northern blotting, and hybridization disruption analysis. Also the probes of the invention can be used to detect SNPs in amplified sequences, such as amplified PCR products generated using the primers described above. For example, in one embodiment a target nucleic acid is first amplified, such as by PCR or strand displacement amplification (SDA), and the amplified double stranded DNA product is then denatured and hybridized with a probe.

In other embodiments double stranded DNA (amplified or not) is denatured and hybridized with a probe of the present invention and then the hybridization complex is subjected to destabilizing or disrupting conditions. By determining the level of disruption energy required wherein the probe has different disruption energy for one allele as compared to another allele, the genotype of a gene at a polymorphic locus can be determined. In one example, there can be lower disruption energy, e.g., melting temperature, for an allele that harbors a cytosine residue at a polymorphic locus, and a higher required energy for an allele with a thymine residue at that polymorphic locus. This can be achieved where the probe has 100% homology with one allele (a perfectly matched probe), but has a single mismatch with the alternative allele. Since the perfectly matched probe is bound more tightly to the target DNA than the mismatched probe, it requires more energy to cause the hybridized probe to dissociate.

In one embodiment the destabilizing conditions comprise an elevation of temperature. The higher the temperature, the greater the degree of destabilization. In another embodiment, the destabilizing conditions comprise subjecting the hybridization complex to a temperature gradient, whereby, as the temperature is increased, the degree of destabilization increases. In an alternative embodiment, the destabilizing conditions comprise treatment with a destabilizing compound, or a gradient comprising increasing amounts of such a compound. Suitable destabilizing compounds include, but are not limited to, salts and urea. Methods of destabilizing or denaturing hybridization complexes are well known in the art, and any such method may be used in accordance with the present invention. For example, methods of destabilizing or denaturing hybridization complexes are taught by Sambrook et al., Molecular Cloning; A Laboratory Manual 2d ed. (1989).

For optimal detection of single-base pair mismatches, it is preferable that there is about a 1° C. to about a 10° C. difference in melting temperature of the probe DNA complex when bound to one allele as opposed to the alternative allele at the polymorphic site. Thus, when the temperature is raised above the melting temperature of a probe:DNA duplex corresponding to one of the alleles, that probe will disassociate.

In one embodiment of the above method, a second (“anchor”) probe can be used. Generally, the anchor probe is not specific to either allele, but hybridizes regardless of what nucleotide is present at the polymorphic locus. The anchor probe does not affect the disruption energy required to disassociate the hybridization complex but, instead, contains a complementary label for using with the first (“sensor”) probe, for example for use in fluorescence resonance energy transfer or “FRET.” A sensor probe acquires energy from the anchor probe once conditions are adequate for hybridization between the target DNA and the anchor and sensor probes. Once hybridization occurs, the anchor probe transfers its florescence energy to the sensor probe, which only will emit a specific wavelength after it has acquired the energy from the anchor probe. Detection of the SNP occurs as the temperature is raised at a predetermined rate, and a reading is acquired from the florescent light emitted. If there is a single base mismatch of the probe and target DNA caused by the presence of the alternative polymorphic nucleotide (i.e. the SNP) the sensor probe will dissociate sooner, or at a lower temperature, since the homology between the genomic DNA and the sensor probe will be less than that of genomic DNA that does not harbor the altered nucleotide or SNP. Thus, there will be a loss of fluorescence that can be detected. Where the probe is designed to bind to the wild-type sequence, the dissociation of the probe from the DNA (i.e. the “melting”) will occur at a lower temperature if the SNP is present, since the stability of the binding of the probe to the SNP is slightly less than for the wild-type sequence. This occurs, obviously, on both chromosomes at the same time, thus yielding either a reading of two identical melting temperatures for a homozygote, or a reading of two different melting temperatures for the heterozygote. For example, where a probe is designed to have the sequence of the C-containing allele of the UASMS1 polymorphism, the probe will dissociate or melt at a lower temperature in DNA samples from individuals that harbor two copies of the polymorphic T-containing allele, than in individuals that harbor two copies of the C-containing allele.

In other embodiments, two different “allele-specific probes” can be used for analysis of a SNP, a first allele-specific probe for detection of one allele, and a second allele-specific probe for the detection of the alternative allele. For example, in one embodiment the different alleles of the UASMS1 ob polymorphism can be detected using two different allele-specific probes, one for detecting the T-containing allele at nucleotide position 207 of the ob gene promoter, and another for detecting the C-containing allele at nucleotide position 207 of the ob gene promoter. In a preferred embodiment an oligonucleotide probe having the sequence of 5′-CTTTCACCTAGTATATCTAG-3′ (SEQ ID NO: 9) is used to detect the T-containing allele, and an oligonucleotide probe having the sequence of 5′-TCTTTCACCTAGTATGTCTAG-3′ (SEQ ID NO: 10) is used to detect the C-containing allele.

In another embodiment the different alleles of the UASMS2 ob polymorphism can be detected using two different allele-specific probes, one for detecting the T-containing allele at nucleotide position 528 of the ob gene promoter, and another for detecting the C-containing allele at nucleotide position 528 of the ob gene promoter. In a preferred embodiment an oligonucleotide probe having the sequence of 5′-AAGCTCTAGAGCCTATGT-3′ (SEQ ID NO: 13) is used to detect the T-containing allele, and an oligonucleotide probe having the sequence of 5′-CAAGCTCTAGAGCCTGTGT-3′ (SEQ ID NO: 14) is used to detect the C-containing allele.

In another embodiment the different alleles of the UASMS3 ob polymorphism can be detected using two different allele-specific probes, one for detecting the G-containing allele at nucleotide position 1759 of the ob gene promoter, and another for detecting the C-containing allele at nucleotide position 1759 of the ob gene promoter. In a preferred embodiment an oligonucleotide probe having the sequence of 5′-CACACATTCCAATCAA-3′ (SEQ ID NO: 17) is used to detect the G-containing allele, and an oligonucleotide probe having the sequence of 5′-CACATTGCAATCAA-3′ (SEQ ID NO: 18) is used to detect the C-containing allele.

In a further embodiment the different alleles of the EXON2-FB ob polymorphism can be detected using two different allele-specific probes, one for detecting the T-containing allele at nucleotide position 305 of exon 2 of the ob gene, and another for detecting the C-containing allele at nucleotide position 305 of exon 2 of the ob gene. In a preferred embodiment an oligonucleotide probe having the sequence of 5′-CCTTGCAGATGGG-3′ (SEQ ID NO: 22) is used to detect the T-containing allele, and an oligonucleotide probe having the sequence of 5′-CCTTGCGGATGGG-3′ (SEQ ID NO: 23) is used to detect the C-containing allele.

Whichever probe sequences and hybridization methods are used, one skilled in the art can readily determine suitable hybridization conditions, such as temperature and chemical conditions. Such hybridization methods are well known in the art. For example, for applications requiring high selectivity, one will typically desire to employ relatively stringent conditions for the hybridization reactions, e.g., one will select relatively low salt and/or high temperature conditions, such as provided by about 0.02 M to about 0.10 M NaCl at temperatures of about 50° C. to about 70° C. Such high stringency conditions tolerate little, if any, mismatch between the probe and the template or target strand, and are particularly suitable for detecting specific SNPs according to the present invention. It is generally appreciated that conditions can be rendered more stringent by the addition of increasing amounts of formamide. Other variations in hybridization reaction conditions are well known in the art (see for example, Sambrook et al., Molecular Cloning; A Laboratory Manual 2d ed. (1989)).

In addition to the SNPs described above, it will be appreciated by those skilled in the art that other DNA sequence polymorphisms of the ob gene may exist within a population. Such natural allelic variations can typically result in about 1-5% variance in the nucleotide sequence of the gene. For example, SEQ ID NO: 2: provides a sequence of a region of the ob gene promoter containing a polymorphism at nucleotide position 207 (i.e. the UASMS1 SNP). It is possible that other polymorphic loci may also exist within this fragment. In addition to naturally-occurring allelic variants of the nucleotide sequence, the skilled artisan will further appreciate that changes can be introduced by mutation into the nucleotide sequence of the nucleotide sequences described herein. Any and all such additional nucleotide variations are intended to be within the scope of the invention. Thus, for example a probe according to the present invention may be designed to bind to a sequence of the ob gene containing not only the UASMS1 polymorphism, but also other SNPs that may occur within the same region.

Moreover, nucleic acid molecules that differ from the sequences of the primers and probes disclosed herein, are intended to be within the scope of the invention. Nucleic acid sequences that are complementary to these sequences, or that are hybridizable to the sequences described herein under conditions of standard or stringent hybridization, and also analogs and derivatives are also intended to be within the scope of the invention. Advantageously, such variations will differ from the sequences described herein by only a small number of nucleotides, for example by 1, 2, or 3 nucleotides.

Nucleic acid molecules corresponding to natural allelic variants, homologues (i.e., nucleic acids derived from other species), or other related sequences (e.g., paralogs) of the sequences described herein can be isolated based on their homology to the nucleic acids disclosed herein, for example by performing standard or stringent hybridization reactions using all or a portion of the sequences of the invention as probes. Such methods for nucleic acid hybridization and cloning are well known in the art.

Similarly, a nucleic acid molecule of the invention may include only a fragment of the specific sequences described. Fragments provided herein are defined as sequences of at least 6 (contiguous) nucleic acids, a length sufficient to allow for specific hybridization of nucleic acid primers or probes, and are at most some portion less than a full-length sequence. Fragments may be derived from any contiguous portion of a nucleic acid sequence of choice. Derivatives and analogs may be full length or other than full length, if the derivative or analog contains a modified nucleic acid or amino acid, as described below.

Derivatives, analogs, homologues, and variants of the nucleic acids of the invention include, but are not limited to, molecules comprising regions that are substantially homologous to the nucleic acids of the invention, in various embodiments, by at least about 70%, 80%, 85%, 90%, 95%, 96%, 97%, 98%, or even 99% identity (with an advantageous identity of 80-99%) over a nucleic acid sequence of identical size or when compared to an aligned sequence in which the alignment is done by a computer homology program known in the art.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present invention is the WU-BLAST (Washington University BLAST) version 2.0 software. WU-BLAST version 2.0 executable programs for several UNIX platforms can be downloaded from ftp://blast.wustl. edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein).

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Alternatively or additionally, the term “homology” or “identity”, for instance, with respect to a nucleotide or amino acid sequence, can indicate a quantitative measure of homology between two sequences. The percent sequence homology can be calculated as (N_{ref}−N_{dij})*100/N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{ref}=8; N_{dif}=2). “Homology” or “identity” can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur & Lipman, Proc Natl Acad Sci USA 1983; 80:726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being considered equal to uracil (U) in RNA sequences. Without undue experimentation, the skilled artisan can consult with many other programs or references for determining percent homology.

The primers and probes described herein may be readily prepared by, for example, directly synthesizing the fragment by chemical means or by introducing selected sequences into recombinant vectors for recombinant production. Methods for making a vector or recombinants or plasmid for amplification of the fragment either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,683; 5,494,807; 5,591,639; 5,589,466; 5,677,178; 5,591,439; 5,552,143; 5,580,859; 6,130,066; 6,004,777; 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450 and 6,312,683; U.S. patent application Serial No. 920,197, filed Oct. 16, 1986; WO 90/01543; WO91/11525; WO 94/16716; WO 96/39491; WO 98/33510; EP 265785; EP 0 370 573; Andreansky et al., Proc. Natl. Acad. Sci. USA 1996; 93:11313-11318; Ballay et al., EMBO J. 1993; 4:3861-65; Felgner et al., J. Biol. Chem. 1994; 269:2550-2561; Frolov et al., Proc. Natl. Acad. Sci. USA 1996; 93:11371-11377; Graham, Tibtech 1990; 8:85-87; Grunhaus et al., Sem. Virol. 1992; 3:237-52; Ju et al., Diabetologia 1998; 41:736-739; Kitson et al., J. Virol. 1991; 65:3068-3075; McClements et al., Proc. Natl. Acad. Sci. USA 1996; 93:11414-11420; Moss, Proc. Natl. Acad. Sci. USA 1996; 93:11341-11348; Paoletti, Proc. Natl. Acad. Sci. USA 1996; 93:11349-11353; Pennock et al., Mol. Cell. Biol. 1984; 4:399-406; Richardson (Ed), Methods in Molecular Biology 1995; 39, “Baculovirus Expression Protocols,” Humana Press Inc.; Smith et al. (1983) Mol. Cell. Biol. 1983; 3:2156-2165; Robertson et al., Proc. Natl. Acad. Sci. USA 1996; 93:11334-11340; Robinson et al., Sem. Immunol. 1997; 9:271; and Roizman, Proc. Natl. Acad. Sci. USA 1996; 93:11307-11312.

Oligonucleotide sequences used as primers or probes according to the present invention may be labeled with a detectable moiety. As used herein the term “sensors” refers to such primers or probes labeled with a detectable moiety. Various labeling moieties are known in the art. Said moiety may be, for example, a radiolabel (e.g., ³H, ¹²⁵I, ³⁵S, ¹⁴C, ³²P, etc.), detectable enzyme (e.g. horse radish peroxidase (HRP), alkaline phosphatase etc.), a fluorescent dye (e.g., fluorescein isothiocyanate, Texas red, rhodamine, Cy3, Cy5, Bodipy, Bodipy Far Red, Lucifer Yellow, Bodipy 630/650-X, Bodipy R6G-X and 5-CR 6G, and the like), a calorimetric label such as colloidal gold or colored glass or plastic (e.g. polystyrene, polypropylene, latex, etc.), beads, or any other moiety capable of generating a detectable signal such as a calorimetric, fluorescent, chemiluminescent or electrochemiluminescent (ECL) signal.

Primers or probes may be labeled directly or indirectly with a detectable moiety, or synthesized to incorporate the detectable moiety. In one embodiment, a detectable label is incorporated into a nucleic acid during at least one cycle of a cyclic polymerase-mediated amplification reaction. For example, polymerases can be used to incorporate fluorescent nucleotides during the course of polymerase-mediated amplification reactions. Alternatively, fluorescent nucleotides may be incorporated during synthesis of nucleic acid primers or probes. To label an oligonucleotide with the fluorescent dye, one of conventionally-known labeling methods can be used (Nature Biotechnology, 14, 303-308, 1996; Applied and Environmental Microbiology, 63, 1143-1147, 1997; Nucleic Acids Research, 24, 4532-4535, 1996). An advantageous probe is one labeled with a fluorescent dye at the 3′ or 5′ end and containing G or C as the base at the labeled end. If the 5′ end is labeled and the 3′end is not labeled, the OH group on the C atom at the 3′-position of the 3′ end ribose or deoxyribose may be modified with a phosphate group or the like although no limitation is imposed in this respect.

Spectroscopic, photochemical, biochemical, immunochemical, electrical, optical or chemical means can be used to detect such labels. The detection device and method may include, but is not limited to, optical imaging, electronic imaging, imaging with a CCD camera, integrated optical imaging, and mass spectrometry. Further, the amount of labeled or unlabeled probe bound to the target may be quantified. Such quantification may include statistical analysis. In other embodiments the detection may be via conductivity differences between concordant and discordant sites, by quenching, by fluorescence perturbation analysis, or by electron transport between donor and acceptor molecules.

In yet another embodiment, detection may be via energy transfer between molecules in the hybridization complexes in PCR or hybridization reactions, such as by fluorescence energy transfer (FET) or fluorescence resonance energy transfer (FRET). In FET and FRET methods, one or more nucleic acid probes are labeled with fluorescent molecules, one of which is able to act as an energy donor and the other of which is an energy acceptor molecule. These are sometimes known as a reporter molecule and a quencher molecule respectively. The donor molecule is excited with a specific wavelength of light for which it will normally exhibit a fluorescence emission wavelength. The acceptor molecule is also excited at this wavelength such that it can accept the emission energy of the donor molecule by a variety of distance-dependent energy transfer mechanisms. Generally the acceptor molecule accepts the emission energy of the donor molecule when they are in close proximity (e.g. on the same, or a neighboring molecule). FET and FRET techniques are well known in the art, and can be readily used to detect the SNPs of the present invention. See for example U.S. Pat. Nos. 5,668,648, 5,707,804, 5,728,528, 5,853,992, and 5,869,255 (for a description of FRET dyes), Tyagi et al. Nature Biotech. vol. 14, p 303-8 (1996), and Tyagi et al., Nature Biotech. vol 16, p 49-53 (1998) (for a description of molecular beacons for FET), and Mergny et al. Nucleic Acid Res. vol 22, p 920-928, (1994) and Wolf et al. PNAS vol 85, p 8790-94 (1988) (for general descriptions and methods fir FET and FRET), each of which is hereby incorporated by reference.

The oligonucleotide primers and probes of the present invention have commercial applications in diagnostic kits for the detection of the UASMS1, UASMS2, UASMS3 and EXON2-FB ob gene SNPs in livestock specimens. A test kit according to the invention may comprise any of the oligonucleotide primers or probes according to the invention. Such a test kit may additionally comprise one or more reagents for use in cyclic polymerase mediated amplification reactions, such as DNA polymerases, nucleotides (dNTPs), buffers, and the like. An SNP detection kit may also include, a lysing buffer for lysing cells contained in the specimen.

A test kit according to the invention may comprise a pair of oligonucleotide primers according to the invention and a probe comprising an oligonucleotide according to the invention. In some embodiments such a kit will contain two allele specific oligonucleotide probes. Advantageously, the kit further comprises additional means, such as reagents, for detecting or measuring the binding or the primers and probes of the present invention, and also ideally a positive and negative control.

The present invention further encompasses probes according to the present invention that are immobilized on a solid or flexible support, such as paper, nylon or other type of membrane, filter, chip, glass slide, microchips, microbeads, or any other such matrix, all of which are within the scope of this invention. The probe of this form is now called a “DNA chip”. These DNA chips can be used for analyzing the SNPs of the present invention. The present invention further encompasses arrays or microarrays of nucleic acid molecules that are based on one or more of the sequences described herein. As used herein “arrays” or “microarrays” refers to an array of distinct polynucleotides or oligonucleotides synthesized on a solid or flexible support, such as paper, nylon or other type of membrane, filter, chip, glass slide, or any other suitable solid support. In one embodiment, the microarray is prepared and used according to the methods and devices described in U.S. Pat. Nos. 5,446,603; 5,545,531; 5,807,522; 5,837,832; 5,874,219; 6,114,122; 6,238,910; 6,365,418; 6,410,229; 6,420,114; 6,432,696; 6,475,808 and 6,489,159 and PCT Publication No. WO 01/45843 A2, the disclosures of which are incorporated by reference in their entireties.

As described in detail above, the present invention provides reagents and methods for the detection of the UASMS1, UASMS2, UASMS3, E2JW and EXON2-FB SNPs and the F279Y SNP of bGHr in DNA samples obtained from individual animals. For example, using the methods of the present invention, one can determine whether a given animal has a cytosine or a thymine at the polymorphic UASMS1 locus (located at nucleotide position 207 of the ob gene promoter). Having used the methods of the invention to determine the genotype of an animal of interest at either the UASMS1, UASMS2, UASMS3, E2JW and/or EXON2-FB a polymorphic loci, it is a further object of the present invention to utilize this genotype information to select and/or group animals according to their genotype.

As described in the Examples, certain alleles of the UASMS1, UASMS2, UASMS3, E2JW and EXON2-FB SNPs, and the F279Y SNP of bGHr, are associated with certain economically important traits such as circulating leptin levels, feed intake, growth rate, body weight, carcass merit and composition, and milk yield. For example, the present invention demonstrates that the T allele of the UASMS2 locus is significantly associated with serum leptin concentration, being lowest in homozygous animals with the CC genotype, intermediate in heterozygous animals with the CT genotype, and highest in homozygous TT animals.

The association of genotypes for SNP in the leptin gene with carcass and meat quality traits in beef cattle was examined, as well as the F279Y SNP of bGHr with performance traits of dairy cattle. Five SNPs (UASMS1, UASMS2, UASMS3, E2JW and EXON2-FB) were genotyped on crossbred bulls, heifers and steers. The measured traits included fat, lean and bone yield (%) by partial rib dissection, grade fat, longissimus muscle (LM) area, hot carcass weight, quality grade, LM intramuscular fat, and tenderness evaluation of LM and semitendinosus muscle. Only four SNPs were analyzed (UASMS1, UASMS2, E2JW and EXON2-FB), because UASMS1 and UASMS3 were completely linked. A univariate mixed inheritance animal model was used to evaluate the association of the SNP genotypes or haplotypes with the traits. The two leptin exon 2 SNPs have been associated with fat and lean yield and grade fat (E2JW, P<0.01; EXON2-FB, P<0.05) and they interacted in their effect on longissimus muscle (LM) tenderness (P<0.01). The leptin promoter SNPs were either not associated with any of the traits (UASMS2) or with fat yield only (UASMS1). Three haplotypes (TCAC, CCAT, TTAC) were at high frequency in the population (88%) and had similar effects on all the traits. Compared to the common haplotypes, one haplotype (CCTT) showed a significantly different effect on fat yield (FATYL), grade fat (GFAT) and lean yield (LEANYL) (P<0.01) and one haplotype (TTTT) on LM tenderness (P<0.03). Therefore, important associations between single nucleotide polymorphisms within the leptin gene with lean yield and tenderness were detected.

Thus in one embodiment, where it is desirable to group animals according to circulating leptin concentration (e.g., for use in food production or for breeding), animals can be selected and grouped according to their genotype at the polymorphic UASMS1 locus. Associations between the genotypes of each of the UASMS1, UASMS2, UASMS3 and EXON2-FB polymorphic loci and various other economically important traits are described in the Examples. Thus, for each of these traits, animals can be grouped according to genotype.

In contrast to the study that described the E2JW SNP (Lagonigro et al., (2003)), evidence of association of E2JW with FATYL, GFAT and LEANYL was found as described in Examples 7-12 below. In the original study, non-significant association of E2JW with percentage of subcutaneous and ultrasound backfat thickness (at 10 months of age) from 169 Holstein-Charolais F₂ bull calves was reported. The same authors also reported no significant association of E2JW with carcass intramuscular fat and marbling score, which agrees with the results of the present study that shows no association of E2JW with either CF or QG. Lagonigro et al., (2003) reported a significant association of E2JW with the average feed intake of bull calves from to months of age, with the AT genotype having higher daily intake than the AA genotype. The contrary findings of the present invention showed, however, that AT animals had lower FATYL and GFAT and higher LEANYL than AA animals, with the expectation that AT animals would have lower feed intake, contrary to the findings of Lagonigro et al. (2003).

Phenotypic and genetic relationships between marbling and tenderness (measured by either shear force evaluation or taste panel) show favorable direction (Bertrand et al.,), indicating that higher marbling is slightly associated with higher tenderness. Increased marbling results in a dilution effect on the connective tissue (collagen) in meat, which aid in the improvement of tenderness.

Analyses of LM tenderness were also carried out adjusting records for either CF or slaughter age through the inclusion of a fixed linear regression on either CF or slaughter age in the model (1). Results were similar to those from the analyses without adjustment. For instance, the probabilities for Wald F-tests for the effects of the joint E2JW/EXON2-FB genotypes on LMAVG were equal to 0.001, adjusting for either CF or slaughter age. The least squares means for E2JW/EXON2-FB genotypes (AA.CC, AA.CT, AATT, AT.CT, and AT.TT) were 4.12 kg, 4.23 kg, 4.50 kg, 3.99 kg and 5.28 kg, adjusting for CF, respectively. The same features adjusting for slaughter age were 4.14 kg, 4.23 kg, 4.49 kg, 3.99 kg and 5.31 kg, respectively.

The E2JW and EXON2-FB polymorphisms are associated with tenderness of LM and they interact in their effect. Individually, these two SNP explain around 22% of the phenotypic variation on tenderness if an additive effect of the E2JW T allele is assumed.

Two SNP in the leptin promoter, UASMS1 and UASMS3, are completely linked in the population and are significantly associated with fat yield. Another leptin promoter polymorphism, UASMS2 is not significantly associated in this population with any carcass and meat quality traits analyzed, which disagrees with two previously reported studies on this polymorphism. Three particular haplotypes (TCAC, CCAT and TTAC) within the leptin gene are highly frequent in the population and do not differ in their effects on carcass and meat quality traits, even though they carry different alleles. This might indicate the effect of other SNPs linked to the four SNP considered in this study or some degree of epistasis among the SNP within the same chromosome.

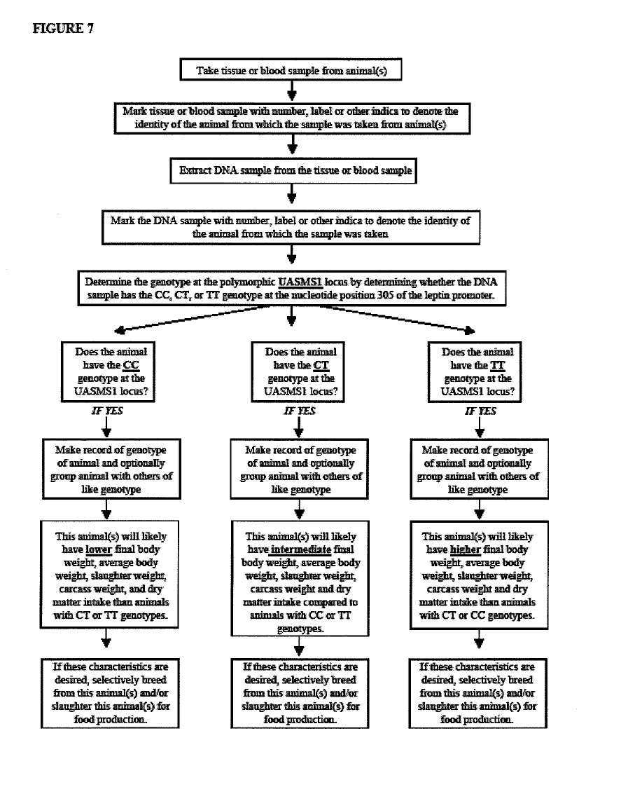

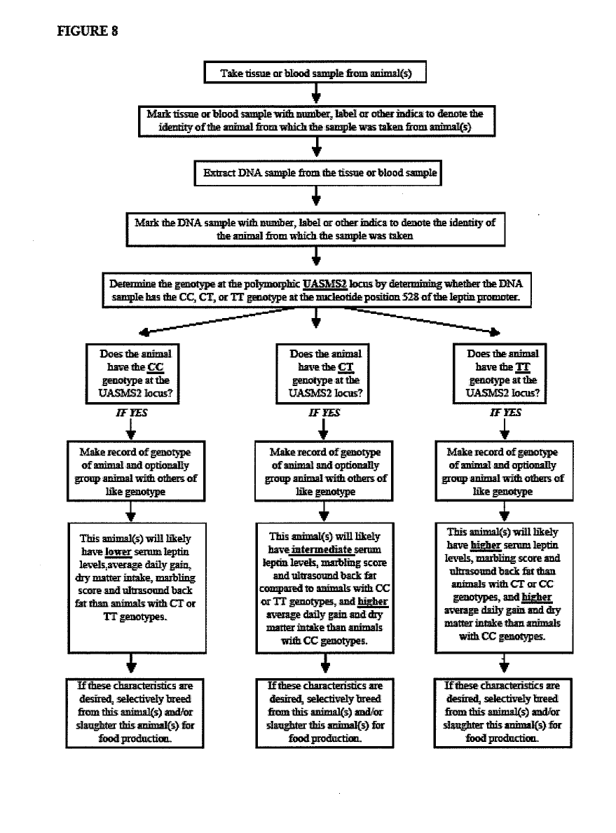

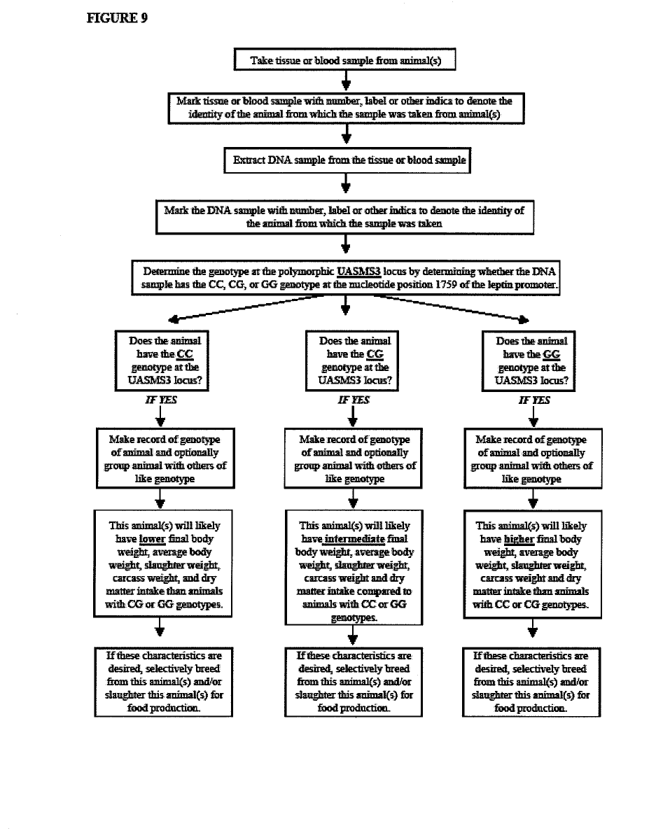

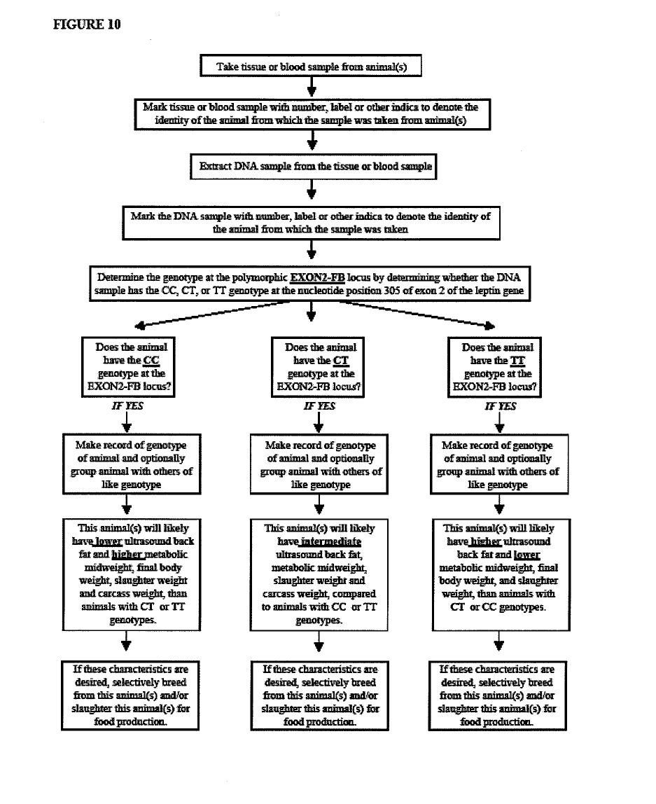

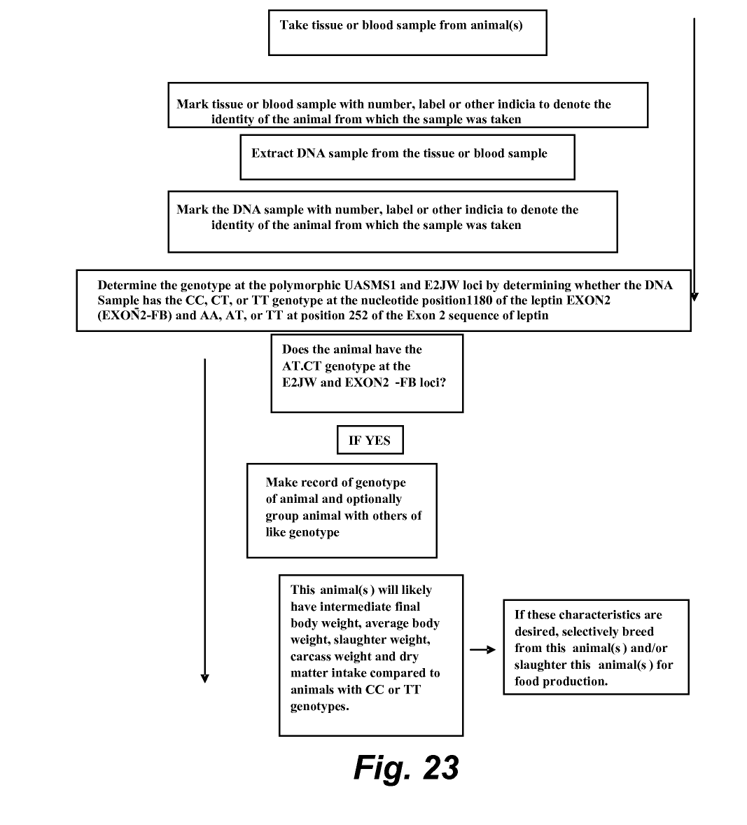

FIGS. 7, 8, 9, 10 and 23 illustrate using flow charts how the animals may be screened for the UASMS1, UASMS2, UASMS3, and EXON2-FB SNPs and the SNP combination E2JW/EXON2-FB respectively, and illustrate how the genotype information may be used to select animals to breed from and/or use for food production. The methods outlined in these flow charts are not intended to be limiting, and those skilled in the art would recognize that various aspects of these methods could be altered without affecting the overall result. FIG. 7-10 illustrate some of the phenotypic characteristics that are associated with each genotype. Other phenotypes that show some level of correlation to each genotype are shown in the Examples section.

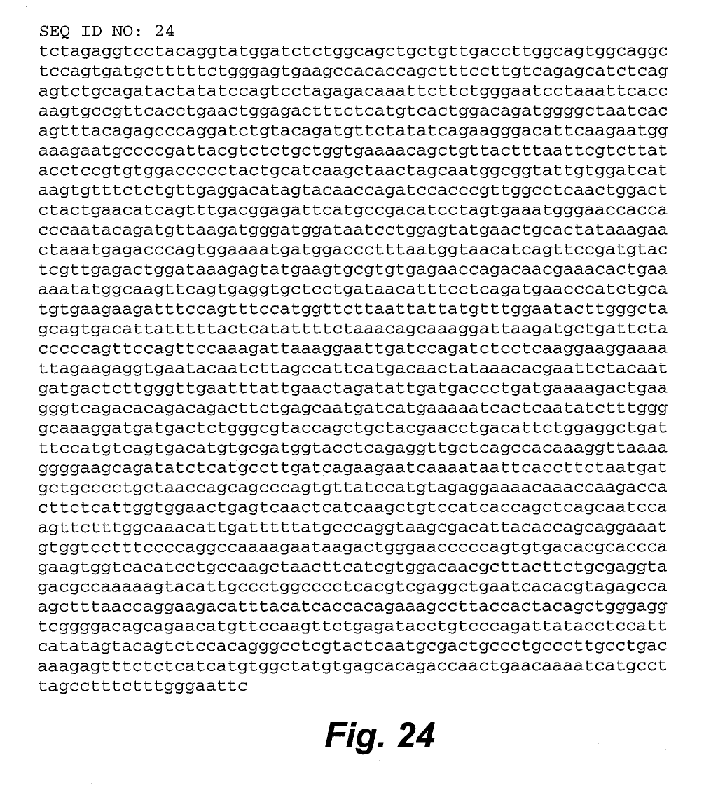

It is further contemplated that the invention may encompass the use of quantitative trait loci (QTLs) other than the bovine leptin gene. For example, a particularly advantageous gene is that encoding the bovine growth hormone receptor (bGHr). In an embodiment wherein the gene of interest is bovine growth hormone receptor (“bGHr”), the bGHr nucleotide sequence can have the sequence corresponding to GenBank Accession No. X70041, or a fragment thereof, and the bGHr amino acid sequence can have the sequence corresponding to Entrez Protein Accession No. CAA49635, or a fragment thereof, the disclosures of which are incorporated by reference in their entireties. Primers for use in the amplification, detection and identification of SNPs associated with desirable traits of the target animals have been described, for example, in Blott et al. Genetics 163: 253-266 (2003) the disclosure of which is incorporated by reference in its entirety. The SNP most advantageously useful in the present invention corresponds to the nucleotide position 854 of the cDNA sequence SEQ ID NO: 24 (GenBank Accession No. X70041) illustrated in FIG. 24, generating a phenylananine-tyrosine amino acid change in exon 8 of the bGHr gene.

Thus, in one embodiment, the present invention provides methods for grouping animals and methods for managing livestock production comprising grouping livestock animals, such as cattle, according to genotype of the UASMS1, UASMS2, UASMS3, E2JW and/or EXON2-FB polymorphic loci. It is a further aspect of the invention that the SNPs of the leptin gene may be combined as indicators and predictors of the quality of livestock prior to their slaughter. In one exemplary embodiment of the invention therefore, the markers UASMS1(3) may be combined with the markers E2JW or EXON2-FB as an indicator of increased tenderness of the meat (as determined by LM shear strength. It is anticipated, therefore, that the methods of the present invention will provide genotype data from one or a combination of markers, and most advantageously the UASMS1, UASMS2, UASMS3, E2JW and/or EXON2-FB markers of the leptin gene locus, that enable the livestock producer to predict with increased reliability the quality of the meat and the animals. The genetic selection and grouping methods of the present invention can be used in conjunction with other conventional phenotypical grouping methods such as grouping animals by visible characteristics such as weight, frame size, breed traits, and the like.

The methods of the present invention provide for selecting cattle having improved heritable traits, and can be used to optimize the performance of livestock herds in areas such as breeding, food consumption, carcass/meat quality and milk production. The present invention provides methods of screening livestock to determine those more likely to develop a desired body condition by identifying the presence or absence of a polymorphism in the ob genes that is correlated with that body condition.

As described above, and in the Examples, there are various phenotypic traits with which the SNPs of the present invention are associated. Each of the phenotypic traits can be tested using the methods described in the Examples, or using any suitable methods known in the art. Using the methods of the invention, a farmer, or feedlot operator, or the like, can group cattle according to each animal's genetic propensity for a desired trait such as circulating leptin levels, feed intake, growth rate, body weight, carcass merit and composition, and milk yield. as determined by SNP genotype, in addition to the present criteria he would ordinarily use for grouping. The cattle are tested to determine homozygosity or heterozygosity with respect to UASMS1, UASMS2, UASMS3, E2JW and EXON2-FB alleles of the ob gene or F279Y of the bGHr gene so that they can be grouped such that each pen contains cattle with like genotypes.

Each pen of animals may then be fed and otherwise maintained in a manner and for a time determined by the feedlot operator to be ideal for meat production prior to slaughter, or to maximize milk production. Thus the farmer or feedlot operator is presented with opportunities for considerable efficiencies. At present, the feeder feeds all his cattle the same, incurring the same costs for each animal, and typically, with excellent management practices, perhaps 40% will grade and receive the premium price for the palatability grade (depending on several other factors, such as age of animal, as we know cattle between 17-24 months of age have increased marbling compared to their younger counterparts. Approximately 55% of cattle are slaughtered at an age under 16 months, and 45% would be slaughtered at an age over 17 months). Of these, a significant number will have excess fat and can receive a reduced yield grade (c.f. Canadian scale for yield grade). The balance of the cattle, 60%, will grade less than AAA, and thus receive a reduced price, although the feedlot costs incurred by the operator are the same. Grouping and feeding the cattle by genotype allows the farmer to treat each group differently with a view to increasing profit.

It is contemplated that, regardless of the desirability and premium paid for any particular meat quality at any given time, providing the farmer with a more uniform group that has a predictable meat quality will provide the farmer with the opportunity to demand and receive a premium, relative to the less uniform groups of cattle presently available.

The methods of the invention are also useful in breeding programs to select for those animals having desirable phenotypes for various economically important traits, such as circulating leptin levels, feed intake, growth rate, body weight, carcass merit and composition, and milk yield. Continuous selection and breeding of animals, such as livestock, that are at least heterozygous and advantageously homozygous for a desirable polymorphism associated with, for example, improved carcass merit, would lead to a breed, line, or population having higher numbers of offspring with improved carcass merit. Thus, farmers can increase the value of their calves by using the methods of the present invention to increase the occurrence of the specific alleles in calves that are associated with economically important traits. Thus, the SNPs of the present invention can be used as selection tools in breeding programs.

One aspect of the invention, therefore, provides a method for sub-grouping animals according to genotype wherein the animals of each sub-group have a similar polymorphism or combination of polymorphisms in the leptin gene comprising (a) determining the genotype of each animal to be subgrouped by determining the presence of a single nucleotide polymorphism or a combination of single nucleotide polymorphisms in the leptin (ob) gene, wherein the single nucleotide polymorphisms are selected from the group consisting of UASMS1, UASMS2, UASMS3, EXON2-FB, and E2JW; and segregating individual animals into sub-groups wherein each animal in a subgroup has a similar polymorphism or combination of polymorphisms in the leptin gene.

In one embodiment of this aspect of the invention, the animal is a bovine and the leptin gene is the bovine leptin gene.

In various embodiments of this aspect of the invention, the combination of single nucleotide polymorphisms of the leptin gene may be selected from the group consisting of UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, and UASMS3/E2JW, and wherein individual animals are segregated into sub-groups depending on whether the animals have, or do not have, the UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, and UASMS3/E2JW single nucleotide polymorphism combinations of the leptin gene.

In one embodiment, the combination of single nucleotide polymorphisms of the leptin gene comprises the markers UASMS1/EXON2-FB, UASMS3/EXON2-FB, EXON2-FB/E2JW, UASMS1/E2JW, or UASMS3/E2JW, and wherein the combination of SNPs indicates an increase in the tenderness of meat.

In another embodiment according to the invention, the combination of single nucleotide polymorphisms of the leptin gene comprises the markers UASMS1/EXON2-FB, UASMS3/EXON2-FB, EXON2-FB/E2JW, UASMS1/E2JW, or UASMS3/E2JW.

In still another embodiment of the invention, the combination of single nucleotide polymorphisms of the leptin gene comprises the markers EXON2-FB/E2JW.

In other embodiments of the invention, the combination of single nucleotide polymorphisms of the leptin gene comprises the markers UASMS1/EXON2-FB or UASMS3/EXON2-FB.

In still other embodiments of the invention, the combination of single nucleotide polymorphisms of the leptin gene comprises the markers UASMS1/E2JW, or UASMS3/E2JW.

In the embodiments of this aspect of the invention, the method may further comprise determining the presence of a single nucleotide polymorphism in the gene encoding a bovine growth hormone receptor.

In one embodiment, the single nucleotide polymorphism in the bovine growth hormone receptor is F279Y, wherein F279Y is a determinant of ribeye area, yield grade and dry material intake.

In the various embodiments of the invention, the combination of single nucleotide polymorphisms is selected from the group consisting of UASMS1/F279Y, UASMS2/F279Y, UASMS3/F279Y, EXON2-FB/F279Y, F279Y/E2JW, UASMS1/UASMS2/F279Y, UASMS1/UASMS3/F279Y, UASMS2/UASMS3/F279Y, UASMS1/EXON2-FB/F279Y, UASMS2/EXON2-FB/F279Y, UASMS3/EXON2-FB/F279Y, EXON2-FB/E2JW/F279Y, UASMS1/E2JW/F279Y, UASMS2/E2JW/F279Y, and UASMS3/E2JW/F279Y.

Another aspect of the invention is a method for identifying an animal having a desirable phenotype relating to certain feed intake, growth rate, body weight, carcass merit and composition, and milk yield, as compared to the general population of animals of that species, comprising determining the presence of a single nucleotide polymorphism or combination of single nucleotide polymorphisms of the animal, wherein the single nucleotide polymorphism is selected from the group consisting of UASMS1, UASMS2, UASMS3, EXON2-FB, E2JW and F279Y, and wherein the combination of single nucleotide polymorphisms is selected from the group consisting of UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, UASMS3/E2JW, UASMS1/F279Y, UASMS2/F279Y, UASMS3/F279Y, EXON2-FB/F279Y, F279Y/E2JW, UASMS1/UASMS2/F279Y, UASMS1/UASMS3/F279Y, UASMS2/UASMS3/F279Y, UASMS1/EXON2-FB/F279Y, UASMS2/EXON2-FB/F279Y, UASMS3/EXON2-FB/F279Y, EXON2-FB/E2JW/F279Y, UASMS1/E2JW/F279Y, UASMS2/E2JW/F279Y, and UASMS3/E2JW/F279Y, and wherein the presence of either the UASMS1, UASMS2, UASMS3 or EXON2-FB single nucleotide polymorphism or the presence of the UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, UASMS3/E2JW, UASMS1/F279Y, UASMS2/F279Y, UASMS3/F279Y, EXON2-FB/F279Y, F279Y/E2JW, UASMS1/UASMS2/F279Y, UASMS1/UASMS3/F279Y, UASMS2/UASMS3/F279Y, UASMS1/EXON2-FB/F279Y, UASMS2/EXON2-FB/F279Y UASMS3/EXON2-FB/F279Y, EXON2-FB/E2JW/F279Y, UASMS1/E2JW/F279Y, UASMS2/E2JW/F279Y, and UASMS3/E2JW/F279Y combinations of single nucleotide polymorphisms is indicative of a desirable phenotype relating to certain feed intake, growth rate, body weight, carcass merit and composition, meat quality, meat tenderness or and milk yield.

Still another aspect of the invention is a composition for the detection of a combination of ob gene polymorphisms selected from the group consisting of UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, UASMS3/E2JW, UASMS1/F279Y, UASMS2/F279Y, UASMS3/F279Y, EXON2-FB/F279Y, F279Y/E2JW, UASMS1/UASMS2/F279Y, UASMS1/UASMS3/F279Y, UASMS2/UASMS3/F279Y, UASMS1/EXON2-FB/F279Y, UASMS2/EXON2-FB/F279Y UASMS3/EXON2-FB/F279Y, EXON2-FB/E2JW/F279Y, UASMS1/E2JW/F279Y, UASMS2/E2JW/F279Y, or UASMS3/E2JW/F279Y, comprising at least two oligonucleotide probes, wherein each oligonucleotide probe is capable of selectively detecting a single polymorphism, and wherein each probe is optionally labeled with a detectable moiety.

One embodiment of this aspect of the invention is an isolated oligonucleotide probe, wherein the detectable moiety is selected from the group consisting of a radiolabel ³H, ¹²⁵I, ³⁵S, ¹⁴C, ³²P, a detectable enzyme, horse radish peroxidase (HRP), alkaline phosphatase, a fluorescent dye, fluorescein isothiocyanate, Texas red, rhodamine, Cy3, Cy5, Bodipy, Bodipy Far Red, Lucifer Yellow, Bodipy 630/650-X, Bodipy R6G-X, 5-CR 6G, a colorimetric label, colloidal gold digoxigenin-dUTP, or biotin.

In one embodiment of the invention, the oligonucleotide is immobilized on a solid support.

Still another aspect of the invention is a method of determining the genotype of an animal at a polymorphic locus of the ob gene comprising: (a) obtaining a DNA sample from the animal; (b) contacting the DNA sample with at least two oligonucleotide primer pairs under conditions suitable for permitting hybridization of the oligonucleotide primers to the DNA sample; (c) enzymatically amplifying specific regions of the ob gene using the primer pairs to form at least two nucleic acid amplification products; (d) contacting the amplification products from step c) with labeled ob gene allele-specific probes, labeled with a detectable moiety, under conditions suitable for permitting hybridization of the labeled allele-specific probes to the amplification products; e) detecting the presence of the amplification products by detecting the detectable moiety of the labeled allele-specific probes hybridized to the amplification products.

In various embodiments of this aspect of the invention, the oligonucleotide primer pairs may be selected from the group consisting of SEQ ID NO: 7 and SEQ ID NO: 8, SEQ ID NO: 11 and SEQ ID NO: 12, SEQ ID NO: 15 and SEQ ID NO: 16, SEQ ID NO: 19 and SEQ ID NO: 20, SEQ ID NO: 21 and SEQ ID NO: 22.

In other embodiments of this method of the invention, the oligonucleotide primer pairs are capable of amplifying regions of the bovine leptin gene having at least one polymorphic nucleotide locus selected from the group consisting of UASMS1, UASMS2, UASMS3, EXON2-FB, and E2JW, or combinations thereof selected from the group consisting of UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, or UASMS3/E2JW. In the embodiments, the genotype may indicate an increase in the tenderness of bovine meat.

In other embodiments of this aspect of the invention, the oligonucleotide primer pairs are capable of amplifying the region of the bovine growth hormone receptor (bGHr) gene having the SNP F279Y.

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

The invention will now be further described by way of the following non-limiting examples.

### EXAMPLES

### Example 1

### Animals and Phenotypic Data Collection

A total of 180 cattle (139 steers and 41 bulls) sired by Angus Charolais or University of Alberta Hybrid bulls were managed and tested for growth and feed efficiency under feedlot conditions. Feed intake was measured for each animal using the GrowSafe® automated feeding system (GrowSafe® Systems Ltd., Airdrie, Alberta, Canada).

Complete performance and efficiency data was available on a total of 150 animals, excluding all the bulls in test two (total of 21 animals) plus nine other animals that died or had to be excluded from the test due to health and other related problems. Weight measurements of all animals were taken weekly. The performance data analyzed include average daily gain (ADG), on-test metabolic midpoint weight (MWT), residual feed intake (RFI), feed conversion ratio (FCR), average daily dry matter intake (DMI), metabolizable energy intake per unit metabolic weight (MEWT^{0.75}), and partial efficiency of growth (PEG). Each animal's ADG during the test was computed as the coefficient of the linear regression of weight (kg) on time (days) using the regression procedure of SAS (SAS Institute, Inc., Cary, N.C., 1999). The MWT of each animal over the test period was computed as the mid-point bodyweight^{0.75}. The total feed intake of each animal over the 70 days test period was used to compute the dry matter intake (DMI) for each animal. Metabolizable energy was calculated as the product of DMI and the dietary energy content (12.14 MJ ME/kg) divided by the metabolic weight of each animal.

Residual feed intake was computed for each animal as the difference between each animal's actual feed intake from predicted expected daily feed intake based on the average daily gain and metabolic weight of each animal over the test period. Feed conversion ratio of each animal was computed as the ratio of average intake on test to average daily gain on test. Partial efficiency of growth (PEG) above maintenance of each animal was computed as the ratio of ADG to the difference between average feed intake and feed intake for maintenance.

Feeding behavior data: The detection of an animal at a feedbunk by the Growsafe system starts a feeding event and ends when the time between the last two readings for the same animal was greater than 300 sec. Detection of an animal within 300 sec was considered to be one continuous feeding event. Feeding event data is then used to compute average Feeding duration (FD) is the differences between average end-time minus start-time. The feeding duration includes time spent in prehension, chewing, backing away from the bunk and chewing, socializing, scratching or licking. Feeding head down time (FHD, on the other hand, primarily includes the time associated with eating and is determined as the average number of detections of an animal during a feeding event times the system detection time of 5.7 sec.

Ultrasound data: Ultrasound measurements of 12/13th rib fat depth, longissimus muscle area and marbling score were taken approximately every 28 days with an Aloka 500V real-time ultrasound with a 17 cm, 3.5-MHz linear array transducer. Each animal had five repeated ultrasound measurements, except for animals removed before the endpoint of test for metabolic studies. In this case the approximate value of the measurement was predicted from the rate of change in that trait from the previous measurements.

Prediction of ultrasound measurements at constant body weight of 500 kg: There was no required weight at slaughter for Canadian Maturity I or young animals (top quality youthful carcasses) under the Canadian Beef Carcass grading system. The average slaughter weight generally ranged between 550 to 600 kg for steers to give an average hot carcass weight of about 350 to 400 kg. The final weights of the animals were below the minimum industry slaughter weight of 500 kg. However, it was desired to determine the final ultrasound measurements of backfat thickness, longissimus thoracis area and marbling score at the time the industry slaughter weight. Regression procedures were used to predict the backfat thickness, marbling score and longissimus thoracis area at a constant body weight of 500 kg.

First, the measurements for each animal (ultrasound backfat thickness, marbling score or longissimus muscle area) recorded on five consecutive periods were regressed on the body weight measured on these above dates for each animal. This yields a regression equation Y=a+b(WT) for each animal, where Y is the trait value to be predicted (backfat, marbling or longissimus thoracis area), a=the intercept of the regression equation; b=the coefficient of regression and WT is the body weight of the animal (in this case set to a constant of 500 kg). Thus the equation was used to predict a value for each trait at a constant body weight of 500 kg for each animal. This resulted in the creation of a new dataset for predicted marbling, backfat or rib eye area. The new dataset was then analyzed to determine the differences between different genotypes of the different markers.

Slaughter and carcass data: Of the 150 animals with complete performance data, 19 of them were bulls that were not sent to slaughter. In addition, 20 animals with extreme phenotypes for RFI were selected for metabolic measurements and no carcass data was collected on these animals. Carcass data was available for only 109 animals. Carcass traits were evaluated according to the Canadian beef carcass grading system. Standard carcass data provided under this system included slaughter weight (final liveweight), carcass weight, average backfat thickness, carcass grade fat, rib eye area, marbling quality or quality grade, marbling level and saleable meat yield. Carcass weight of each animal was determined as the weight of the left and right halves of the carcass after a 24 hr chill at −4° C. Carcass grade fat was measured at the 12/13th rib of each carcass. Average backfat thickness was measured at two different locations along the rib eye muscle other than between the 12 and 13^{th} ribs. Carcass quality grade (A, AA, AAA or prime=4, 3, 2, 1 respectively) were decided according to the following criteria: animal must be physiologically less than 30 months old; meat must be bright red, firm and fine grained; muscling must range from good (with no deficiencies) to excellent; gradefat must be firm and white (or amber) and not less than 2 mm at the site of measurement ( 12/13^{th} rib).

To score A, AA, AAA or prime is not directly dependent on the marbling level. Associated with each of these quality grades is a score for marbling level (ranges from 0 to 90 such as A0, A50, AA10, AAA0, AAA40 etc). To obtain a quantitative value for marbling therefore, the quality grade and marbling level of each graded carcass are combined to compute a value for marbling score according to the equation: marbling score=(QG+ML)/100, where QG is the quality grade (100, 200, 300 and 400 for A, AA, AAA, and prime, respectively) and ML is marbling level and ranges from 0 to 90 in units of 10. Marbling score is a measure of intramuscular fat of the ribeye muscle and can be classified as 1 to <2 units=trace marbling (Canada A quality grade); 2 to <3 units=slight marbling (Canada AA quality grade); 3 to <4 units=small to moderate marbling (Canada AAA quality grade) and ≧4 units=slightly abundant or more marbling (Canada Prime). Lean meat yield is an estimate of saleable meat and was calculated according to the equation: Lean meat yield %=57.96+(0.202×L. thoracis area, cm²)−(0.027× warm carcass weight, kg)−(0.703× average backfat thickness, mm). The lean meat yield of the carcass may be used to assign a grade (yield grade) to each animal according to Y1=≧59%, Y2=54 to <59% and Y3=<54%.

### Example 2

### Blood Sampling, DNA Extraction and SNP Detection

Blood samples were collected from each animal at start of the feed intake test from which genomic DNA was extracted using a modified saturated salt phenol/chloroform procedure (Sambrook et al. (2001) Molecular Cloning: A Laboratory Manual, 3rd ed., Cold Spring Harbor Press). Identification of polymorphisms in the bovine leptin promoter utilized SEQ ID NO: 1 (GenBank accession number AB070368, Taniguchi et al., UBMB Life. 2002 February; 53(2):131-5). Genomic DNA from a panel of 16 animals was amplified by polymerase chain reaction using forward and reverse primers designed to cover the entire bovine leptin promoter region. The PCR products from each animal were sequenced on a Beckman CEQ 8000 Genetic Analysis System (Beckman Coulter Instruments, Inc.). Sequence data for each animal were analyzed to identify putative single nucleotide polymorphisms.

The analysis identified three new single nucleotide polymorphisms (SNPs), namely UASMS1, UASMS2 and UASMS3 located, respectively at positions 207 (C/T substitution), 528 (C/T substitution) and 1759 (C/G substitution) (Numbering is that of SEQ ID NO: 1, GenBank accession number AB070368). The exon 2 SNP identified by Buchanan et al. (Genet Sel Evol. 2002 January-February; 34(1):105-16) is located at position 305 (C/T missence mutation) (GenBank accession No. AY138588). The genotyping of each leptin gene-specific polymorphism was carried out using the 5′ nuclease allelic discrimination assay on an ABI PRISM™ 7700 sequence detector (Applied Biosystems Inc.). Forward and reverse primers (Table 1) were designed to amplify each polymorphism using genomic DNA from each animal. Additionally, two ABI TaqMan fluorogenic probes (with a different reporter dye on each probe) were designed to target two alleles of each SNP (Table 1).

TABLE 1
Position, primer and probe information for genotyping each polymorphism

| | | | |
|---|---|---|---|
| SNP | Position^{a} | Forward Primer | Forward Primer |

| | | | |
|---|---|---|---|
| UASMS1^{a} | 207 (C/T) | ggcacaatcctgtgtattggtaaga | ggcacaatcctgtgtattggtaaga |
| UASMS2^{a} | 528 (C/T) | aggtgcccagggactca | aggtgcccagggactca |
| UASMS3^{a} | 1759 (C/G) | atgtatattttggtgtgagagtgtgtgt | atgtatattttggtgtgagagtgtgtgt |
| EXON2-FB^{b} | 305 (C/T) | ggctttggccctatctgtcttac | ggctttggccctatctgtcttac |

| | | | |
|---|---|---|---|
| SNP | Position^{a} | Probe 1^{c} | Probe 2^{c} |

| | | | |
|---|---|---|---|
| UASMS1^{a} | 207 (C/T) | Ctttcacctagtatatctag | tctttcacctagtatgtctag |
| UASMS2^{a} | 528 (C/T) | Caagctctagagcctgtgt | aagctctagagcctatgt |
| UASMS3^{a} | 1759 (C/G) | Cacacattccaatcaa | cacattgcaatcaa |
| EXON2-FB^{b} | 305 (C/T) | Ccttgcagatggg | ccttgcggatggg |

^{a}Positions are designated according to SEQ ID NO: 1 GenBank accession number AB070368.
^{b}Position is designated according to SEQ ID NO: 5 GenBank accession number AY138588.
^{c}Nucleotides in bold target the specific alleles of the SNP.

A subset of the genotyped animals was sequenced across each polymorphism and the sequence results were used to confirm the genotypes obtained by discrimination assays. In addition to the experimental herd, a total of 160 animals from five commercial lines of relatively unrelated cattle (BeefBooster genetic selection lines M1, M2, M3, M4, and TX) were also genotyped and the allele frequencies of the SNPs were determined in these animals. Foundation breed(s) were Angus for M1, Hereford for M2, various small breeds for M3, Limousin and Gelbvieh for M4, and Charolais for TX (Kress et al., J Anim Sci. 1996 October; 74(10):2344-8).

Chi-square tests were used to examine the genotype frequencies of each polymorphism for deviations from Hardy-Weinberg equilibrium for both the experimental and commercial populations. Differences among the various selection lines of the commercial herd in allele frequencies of the polymorphisms were also tested by chi-square analyses using the Categorical Model Procedure of SAS (SAS Institute, Inc., Cary, N.C., 1999). Single marker associations were then carried out to evaluate the relationship of the different marker genotypes of each marker on serum leptin concentration, growth rate, body weight, feed intake, feed efficiency and ultrasound traits. The data were analyzed using PROC MIXED of SAS (SAS Institute, Inc., Cary, N.C., 1999). The statistical model used included fixed effects of SNP genotype, test group (one and two), sex of animal (bull and steer) and random animal additive effects. Animal was fitted as a random effect to account for background genes. Start weight of animal on test, age of dam or age of animal on test were included in the model as linear covariates. The model used to analyze the carcass data was similar to that of the live animal data but excluded the fixed effects of sex as only steers were sent to slaughter. Associations between different polymorphisms and carcass quality grade were tested by chi-square analyses using the Categorical Model Procedure of SAS (SAS Institute, Inc., Cary, N.C., 1999).

Additive genetic effects were estimated for traits that were significantly different (P<0.10) between animals with different SNP genotypes. Significant additive genetic (a) effects were computed by subtracting the solution of the estimate for the trait effect of the two homozygote genotypes. We also estimated dominance deviation (d) as the deviation of the CT genotypic value from the midpoint between the TT and CC genotypic values.

### Example 3

### Genotype and Allele Frequencies

Tables 2 and 3 show the genotype frequencies and chi-square tests of Hardy-Weinberg equilibrium for the different polymorphisms in the experimental and commercial populations, respectively. Observations of the genotypes revealed that all animals that had genotypes CC, CT or TT of UASMS1 also had genotypes CC, CG or GG of UASMS 3, respectively. Thus, the two polymorphisms were in complete linkage disequilibrium and were designated together as UASMS1-3. The T-G alleles of UASMS1-3 were 59% each in the experimental population and the T alleles of UASMS2 were 21% and EXON2-FB 44%. Similarly, the frequencies of the T-G or T alleles of UASMS1-3, UASMS2 and EXON2-FB were 48%, 20% and 53%, respectively, in the commercial population. Chi-square analyses between observed and expected genotypes showed that the frequencies of all the genotypes of all three polymorphisms did not deviate significantly from Hardy-Weinberg proportions in both populations (P>0.10).

TABLE 2
Genotype frequencies and chi-square tests of Hardy-Weinberg equilibrium
of the three markers in the experimental population

| Polymorphism | CC/CC | CT/CG | TT/GG | TOTAL | % T-G | Chi-square^{z} | p-value^{y} |
|---|---|---|---|---|---|---|---|
| UASMS1-3 | 33 | 82 | 65 | 180 | 0.59 | 0.63 | 0.73 |
| Polymorphism | CC | CT | TT | TOTAL | % T | Chi-square | p-value |
| UASMS2 | 113 | 58 | 9 | 180 | 0.21 | 0.19 | 0.91 |
| EXON2-FB | 59 | 84 | 37 | 180 | 0.44 | 0.50 | 0.78 |

^{z}Degree of deviation of observed genotype frequencies from expectations
^{y}Probability of a significant chi-square value.

TABLE 3
Genotype frequencies and chi-square tests of Hardy-Weinberg equilibrium
of the three markers in the commercial population

| Polymorphism | CC/CC | CT/CG | TT/GG | TOTAL^{x} | % T-G | Chi-square^{z} | P value^{y} |
|---|---|---|---|---|---|---|---|
| UASMS1-3 | 41 | 84 | 35 | 160 | 0.48 | 0.42 | 0.81 |
| Polymorphism | CC | CT | TT | TOTAL | % T | Chi-square | p-value |
| UASMS2 | 100 | 55 | 5 | 160 | 0.20 | 0.61 | 0.74 |
| EXON2-FB | 32 | 86 | 43 | 161 | 0.53 | 0.87 | 0.65 |

^{z}Degree of deviation of observed genotype frequencies from expectations
^{y}Probability of a significant chi-square value.
^{x}The total population size is 162 animals. Two samples failed to amplify for UASMS1, 2 and 3 and one sample failed to amplify for EXON2-FB.

Table 4 shows the frequencies of the different polymorphisms in the different strains of the commercial population. Frequencies of the T-G alleles of UASMS1-3 differed among the different lines of the commercial population (P<0.05, χ²=9.17) and were lower in the M1 line (Angus) compared to TX (Charolais) (P<0.004, χ²=8.10), M2 (Hereford) (P<0.10, χ²=2.86), M3 (various small breeds) (P<0.02 χ²=5.48) and M4 (Gelbvieh and Limousin) (P<0.04, χ²=4.10).

TABLE 4
Genotype and allele frequencies of the various markers
in five strains of a commercial population of cattle

| | | UASMS1-3 | | | | UASMS2 | | | | EXON2-FB | | | |
|---|---|---|---|---|---|---|---|---|---|---|---|---|---|
| Line | Animals | CC/CC | CT/CG | TT/GG | T-G allele | CC | CT | TT | T allele | CC | CT | TT | T allele |
| M1 | 31 | 13 | 16 | 2 | 0.32 ^{a} | 24 | 7 | 0 | 0.11 ^{a} | 2 | 16 | 14 | 0.69^{a} |
| M2 | 33 | 8 | 19 | 6 | 0.47 ^{b} | 19 | 11 | 3 | 0.26 ^{b} | 7 | 15 | 11 | 0.56^{ab} |
| M3 | 31 | 7 | 15 | 9 | 0.53 ^{b} | 18 | 12 | 1 | 0.23 ^{b} | 6 | 18 | 7 | 0.52^{b} |
| M4 | 33 | 7 | 19 | 7 | 0.50 ^{b} | 23 | 9 | 1 | 0.17^{ab} | 6 | 22 | 5 | 0.48^{b} |
| TX | 32 | 6 | 15 | 11 | 0.58 ^{b} | 16 | 16 | 0 | 0.25 ^{b} | 11 | 15 | 6 | 0.42^{b} |

^{a, b, c} Allele frequencies of UASMS1-3 (P = 0.01, χ² = 9.17), UASMS2 (P < 0.05, χ² = 5.71) and EXON2-FB (P < 0.04, χ² = 9.93) in columns followed by different superscripts are different.

Frequency of the T allele of UASMS2 differed among selection lines (P<0.05, χ²=5.71) and was higher for M1 compared to M2 (P<0.05, χ²=4.19), M3 (P<0.10, χ²=2.71) and TX (P<0.05, χ²=3.79). Differences in allele frequency of UASMS2 in the other strains were not significant (P>0.10). There were differences in allele frequencies of EXON2-FB among selection lines of the commercial population (P<0.041, χ²=9.93). The Angus-based selection line (M1) had a higher frequency of the T allele of EXON2-FB compared to the lines based on (Gelbvieh and Limousin (M4) (χ²=5.41, P<0.05) and Charolais (TX) (χ²=P<0.01) and tended to be higher than line based on various small breeds (M3) (χ²=3.82, P<0.10), but not Hereford (M2) (P>0.10). The allele frequency of EXON2-FB did not differ among the other selection lines of the commercial population (P>0.10).

### Example 4

### Associations of UASMS1-3 with Various Phenotypic Traits

Table 5 and shows the effect of different genotypes of UASMS1-3 on measures of serum leptin concentration, performance, feed efficiency and feeding behavior in the experimental population. Metabolic weight was higher (P<0.01) for animals with genotype TT-GG than for CC-CC (additive effect, a=−5.35±1.65 kg^{0.75}). Average daily gain tended to be higher (P<0.10) for animals with genotype TT-GG than for animals with genotype CC-CC (additive effect, a=−0.12±0.04 kg d⁻¹). Dry matter intake was significantly higher (additive effect, a=−0.88±0.24 kg d⁻¹) (P=0.001) and metabolizable energy per metabolic weight tended to differ (P<0.10) [additive effect, a=−49.06±23.60 KJ (kg^{0.75}d)⁻¹] among animals with different genotypes of UASMS1-3. However, serum leptin concentration, feed conversion ratio, residual feed intake and partial efficiency of growth did not show any significant associations with genotypes of UASMS1-3 (P>0.10). For the feeding behavior traits, feeding duration was different (P=0.04) (additive effect, a=−7.66±2.58 min d⁻¹,) among animals with different genotypes of UASMS1-3. On the other hand, feeding frequency tended to be lower (P<0.10) (additive effect, a=3.32±1.07 events d⁻¹,) for animals with genotype TT-GG than for CC-CC.

TABLE 5
Effect of different genotypes of UASMS1-3 (least-squares means ± standard error)
on measures of serum leptin, performance, efficiency and feeding behavior

| | UASMS1-3 Marker genotype^{z} | | | |
|---|---|---|---|---|
| Trait | CC-CC | CT-CG | TT-GG | P value^{y} |
| Number of animals | 27 | 68 | 55 | |
| Serum leptin, performance and efficiency | | | | |
| Serum leptin level, ng ml⁻¹ | 12.48 ± 1.41 | 12.13 ± 0.96 | 13.26 ± 1.00 | 0.60 |
| Metabolic mid-weight, kg^{.75} | 83.79 ± 1.61 | 85.62 ± 1.10 | 89.14 ± 1.13 | 0.002 |
| Average daily gain, kg d⁻¹ | 1.29 ± 0.05 | 1.38 ± 0.04 | 1.42 ± 0.04 | 0.08 |
| Residual feed intake, kg d⁻¹ | −0.53 ± 0.59 | −0.37 ± 0.57 | −0.20 ± 0.58 | 0.25 |
| Feed conversion ratio | 6.04 ± 0.18 | 5.95 ± 0.13 | 6.11 ± 0.13 | 0.57 |
| Dry matter intake, kg d⁻¹ | 7.17 ± 0.23 | 7.59 ± 0.16 | 8.05 ± 0.16 | 0.001 |
| ME intake, KJ kg^{0.75} d⁻¹ | 1034.3 ± 24.1 | 1073.26 ± 16.3 | 1093.4 ± 24.1 | 0.07 |
| Partial Efficiency of growth, | 0.34 ± 0.01 | 0.34 ± 0.01 | 0.32 ± 0.01 | 0.15 |
| Feeding Behavior | | | | |
| Feeding duration, min d⁻¹ | 50.25 ± 2.27 | 52.66 ± 1.55 | 56.27 1.61 | 0.04 |
| Head down time, min d⁻¹ | 34.72 ± 2.25 | 36.76 ± 1.53 | 38.41 ± 1.59 | 0.29 |
| Feeding frequency, events d⁻¹ | 34.06 ± 1.07 | 31.85 ± 0.92 | 30.74 ± 1.01 | 0.08 |

^{z}UASMS1-3 are located at positions 207 (C/T substitution) and 1759 (C/G substitution) in the bovine leptin promoter according to SEQ ID NO: 1 (AB070368)
^{y}P value = probability of differences among different marker genotypes.

Table 6 shows the body weight, ultrasound and carcass measurements of animals with different UASMS1-3 genotypes. Average body weight (additive effect, a=−29.73±10.49 kg), final live weight (additive effect, a=−33.39±11.80) (P<0.01), slaughter weight (additive effect, a=37.07±13.79 kg) and carcass weight (additive effect, a=−18.49±8.59 kg) (P=0.01) were higher in animals with the TT-GG than for CC-CC genotype of UASMS1-3. With the exception of final ultrasound backfat thickness, which was higher in animals with genotypes TT-GG than for CC-CC (P<0.05), there were no differences among genotypes in the different ultrasound measurements (P>0.10). In addition, carcass grade fat, backfat thickness, longissimus muscle area, marbling score and lean meat yield did not differ among different UASMS1-3 genotypes. Categorical data analysis of the carcass grades (A, AA, AAA) among genotypes of UASMS1-3 showed no significant associations between quality grade and genotypes (χ²=1.37, P=0.50) (Table 11).

TABLE 6
Effect of different genotypes of UASMS1-3 (least-squares means ± standard error)
on measures of body weight, ultrasound and carcass merit of hybrid cattle
UASMS1-3 Marker genotype^{z}

| Trait | CC | CT | TT | P value^{y} |
|---|---|---|---|---|
| Number of animals | 27 | 68 | 55 | |
| Weight and ultrasound | | | | |
| Initial Measurements (Jan 10) | | | | |
| Body weight, kg | 335.24 ± 8.88 | 339.51 ± 6.09 | 355.12 ± 6.25 | 0.03 |
| Ultrasound backfat, mm | 5.54 ± 0.53 | 5.10 ± 0.47 | 5.13 ± 0.49 | 0.15 |
| Ultrasound marbling score | 4.25 ± 0.33 | 4.34 ± 0.32 | 4.31 ± 0.33 | 0.68 |
| Longissimus thoracis area, cm² | 64.05 ± 1.15 | 62.88 ± 0.79 | 61.95 ± 0.82 | 0.22 |
| Final Measurements (May 01) | | | | |
| Body weight, kg | 477.46 ± 11.60 | 485.24 ± 7.96 | 510.86 ± 8.16 | 0.005 |
| Ultrasound backfat | 5.84 ± 0.91 | 5.41 ± 0.84 | 6.45 ± 0.88 | 0.04 |
| Ultrasound marbling score | 4.56 ± 0.12 | 4.63 ± 0.08 | 5.69 ± 0.09 | 0.61 |
| Longissimus thoracis area, cm² | 74.71 ± 1.35 | 73.22 ± 0.92 | 73.45 ± 0.96 | 0.53 |
| Average Measurements^{x} | | | | |
| Body weight, kg | 402.90 ± 10.24 | 412.35 ± 7.02 | 432.64 ± 6.20 | 0.01 |
| Ultrasound backfat | 5.72 ± 0.61 | 5.26 ± 0.55 | 5.83 ± 0.57 | 0.17 |
| Ultrasound marbling score | 4.39 ± 0.10 | 4.47 ± 0.07 | 4.67 ± 0.08 | 0.76 |
| Longissimus thoracis area, cm² | 69.32 ± 0.87 | 68.02 ± 0.59 | 67.93 ± 0.62 | 0.26 |
| Carcass Traits | | | | |
| Number of animals | 22 | 49 | 38 | |
| Slaughter weight, kg | 490.6 ± 10.9 | 501.2 ± 7.3 | 527.65 ± 8.31 | 0.01 |
| Carcass weight, kg | 287.8 ± 6.8 | 286.62 ± 4.53 | 306.31 ± 5.18 | 0.01 |
| Grade Fat, mm | 9.52 ± 0.71 | 8.11 ± 0.48 | 9.29 ± 0.54 | 0.15 |
| Ave. Backfat, mm | 10.96 ± 0.75 | 9.67 ± 0.50 | 10.81 ± 0.57 | 0.21 |
| Carcass marbling score | 2.32 0.38 | 2.11 ± 0.38 | 2.29 ± 0.39 | 0.18 |
| L. thoracis area, cm² | 75.45 ± 1.39 | 76.58 ± 0.92 | 76.63 ± 1.09 | 0.77 |
| Lean meat yield, % | 57.52 ± 0.75 | 58.86 ± 0.50 | 57.93 ± 0.59 | 0.25 |

^{z}UASMS1-3 polymorphism are located at positions 207 (C/T substitution) and 1759 (C/G substitution) in the bovine leptin promoter according to SEQ ID NO: 1 (AB070368)
^{y}P value = probability of differences among different marker genotypes.
^{x}Average of five measurements taken between January 10 and May 01 at approximately monthly intervals

### Example 5

### Associations of UASMS2 with Various Phenotypic Traits

The effect of different genotypes of UASMS2 on measures of serum leptin concentration, performance, feed efficiency and ultrasound and carcass merit are presented in Tables 7 and 8. The T allele of UASMS2 was highly significantly associated with serum leptin concentration (P<0.0001), and was higher for animals with genotype TT than for CC (additive effect, a=−11.79±2.76 ng ml⁻¹). Serum leptin was also higher (P=0.04) in CT animals than in CC animals (dominance deviation, d=−3.38±1.81 ng ml⁻¹). Metabolic weight differed among genotypes (P<0.05) and was higher for animals with genotype TT than for CC (additive effect, a=−6.01±2.50 kg^{0.75}). Average daily gain was significantly different (P<0.01) among genotypes and was higher for animals with genotype TT than for animals with genotype CC (additive effect, a=−0.15±0.04 kg d⁻¹).

TABLE 7
Effect of different genotypes of UASMS2 (least-squares
means ± standard error) on measures of serum leptin,
performance, efficiency and feeding behavior hybrid cattle
UASMS2 Marker genotype^{z}

| Trait | CC | CT | TT | P value^{z} |
|---|---|---|---|---|
| Number of animals | 99 | 45 | 6 | |
| Serum leptin, performance | | | | |
| and efficiency | | | | |
| Serum leptin level, ng ml⁻¹ | 11.92 ± 0.93 | 14.43 ± 1.24 | 23.71 ± 2.80 | <0.0001 |
| Metabolic mid- weight, kg^{.75} | 85.77 ± 1.09 | 88.19 ± 1.20 | 92.14 ± 3.04 | 0.03 |
| Average daily gain, kg d⁻¹ | 1.32 ± 0.03 | 1.47 ± 0.04 | 1.46 ± 0.10 | 0.002 |
| Residual feed intake, kg d⁻¹ | −0.71 ± 0.23 | −0.41 ± 0.23 | −0.95 ± 0.40 | 0.09 |
| Feed conversion ratio | 6.06 ± 0.12 | 5.95 ± 0.13 | 5.82 ± 0.34 | 0.63 |
| Dry matter intake, kg d⁻¹ | 7.44 ± 0.15 | 8.13 ± 0.17 | 7.89 ± 0.43 | 0.001 |
| ME intake, KJ kg^{0.75} d⁻¹ | 1061.1 ± 15.9 | 1110.1 ± 18.2 | 1047.3 ± 48.9 | 0.04 |
| Partial Efficiency of growth, | 0.34 ± 0.01 | 0.33 ± 0.01 | 0.36 ± 0.02 | 0.64 |
| Feeding Behavior | | | | |
| Feeding duration, min d⁻¹ | 49.69 ± 3.39 | 54.89 ± 3.31 | 49.96 ± 5.39 | 0.02 |
| Head down time, min d⁻¹ | 34.26 ± 3.17 | 37.17 ± 3.10 | 29.84 ± 5.11 | 0.01 |
| Feeding frequency, events d⁻¹ | 33.12 ± 1.87 | 30.86 ± 1.86 | 28.64 ± 3.34 | 0.09 |

^{z}UASMS2 polymorphism is a C/T substitution located at position 528 of the bovine leptin promoter according to SEQ ID NO: 1 (AB070368)
^{y}P value = probability of differences among different marker genotypes.

TABLE 8
Effect of different genotypes of UASMS2 (least-squares means ± standard error)
on measures of body weight, ultrasound and carcass merit of hybrid cattle
UASMS2 Marker genotype^{z}

| Trait | CC | CT | TT | P value^{y} |
|---|---|---|---|---|
| Number of animals | 99 | 45 | 6 | |
| Body weight and ultrasound | | | | |
| Initial Measurements (Jan 10) | | | | |
| Body weight, kg | 339.93 ± 5.95 | 352.53 ± 6.54 | 363.26 ± 16.59 | 0.01 |
| Ultrasound backfat, mm | 4.38 ± 0.25 | 4.61 ± 0.27 | 5.64 ± 0.68 | 0.15 |
| Ultrasound marbling score | 4.32 ± 0.08 | 4.46 ± 0.08 | 4.56 ± 0.21 | 0.15 |
| Longissimus thoracis area, cm² | 62.97 ± 0.76 | 62.17 ± 0.82 | 60.13 ± 2.13 | 0.32 |
| Final Measurements (May 01) | | | | |
| Body weight, kg | 488.33 ± 7.86 | 502.86 ± 8.64 | 530.35 ± 21.93 | 0.07 |
| Ultrasound backfat | 5.20 ± 0.35 | 6.43 ± 0.38 | 9.51 ± 0.96 | <0.0001 |
| Ultrasound marbling score | 4.61 ± 0.09 | 4.79 ± 0.10 | 5.52 ± 0.25 | 0.001 |
| Longissimus thoracis area, cm² | 74.03 ± 0.09 | 72.64 ± 0.10 | 68.43 ± 2.48 | 0.05 |
| Average Measurements^{x} | | | | |
| Body weight, kg | 413.34 ± 6.93 | 427.65 ± 7.62 | 443.68 ± 19.32 | 0.10 |
| Ultrasound backfat | 4.83 ± 0.26 | 5.43 ± 0.28 | 7.12 ± 0.73 | 0.003 |
| Ultrasound marbling score | 4.41 ± 0.08 | 4.58 ± 0.08 | 5.02 ± 0.21 | 0.006 |
| Longissimus thoracis area, cm² | 68.37 ± 0.57 | 67.77 ± 0.62 | 64.34 ± 1.60 | 0.04 |
| Predicted @ 500 kg BW | | | | |
| Ultrasound backfat | 5.34 ± 0.32 | 6.21 ± 0.34 | 8.65 ± 0.88 | 0.0002 |
| Ultrasound marbling score | 4.57 ± 0.40 | 4.77 ± 0.40 | 5.36 ± .46 | 0.002 |
| Longissimus thoracis area, cm² | 74.65 ± 0.79 | 73.06 ± 0.86 | 70.21 ± 2.19 | 0.05 |
| Carcass data | | | | |
| Number of animals | 76 | 29 | 4 | |
| Slaughter weight, kg | 500.9 ± 6.0 | 516.7 ± 9.9 | 537.27 ± 26.2 | 0.20 |
| Carcass weight, kg | 290.6 ± 3.8 | 299.4 ± 6.2 | 295.9 ± 16.5 | 0.48 |
| Grade Fat, mm | 8.34 ± 0.43 | 9.54 ± 0.70 | 10.91 ± 1.84 | 0.16 |
| Ave. Backfat, mm | 9.76 ± 0.44 | 11.50 ± 0.71 | 12.09 ± 1.92 | 0.08 |
| Carcass marbling score | 2.26 ± 0.07 | 2.42 ± 0.11 | 2.71 ± 0.30 | 0.20 |
| L. thoracis area, cm² | 76.08 ± 0.75 | 77.30 ± 1.22 | 74.63 ± 3.32 | 0.61 |
| Lean meat yield, % | 58.62 ± 0.41 | 57.39 ± 0.65 | 56.90 ± 1.77 | 0.22 |

^{z}UASMS2 polymorphism is a C/T substitution located at position 528 of the bovine leptin promoter according to SEQ ID NO: 1 (AB070368)
^{y}P value = probability of differences among different marker genotypes.
^{x}Average of five measurements taken between January 10 and May 01 at approximately monthly intervals

Dry matter intake was significantly different (P=0.001) among genotypes of UASMS2 and was higher in animals with TT compared to CC (additive effect, a=−0.45±0.19 kg d⁻¹) and CT compared to CC (dominance effect, d=−0.69±0.26 kg d⁻¹). Metabolizable energy per metabolic weight also differed among genotypes of UASMS2 (P=0.04) and was higher in CT compared to TT or CC (dominance deviation, d=−56.11±25.24 KJ (kg^{0.75} d)⁻¹ UASMS2. The higher DM intake of animals with the T allele observed in this study is surprising as it would generally be expected that the animals with higher body fat and significantly higher serum leptin would have decreased feed consumption. It may be argued that this result may be due to the fact that there were only very few animals available with genotype TT for comparison (as seen by the high standard errors associated with the trait values of TT animals). However, the results also showed that feed intake was higher in heterozygous animals, indicating that the T allele of UASMS2 is in fact associated with increased feed intake.

Recent data from dairy cows (Liefers et al., Mamm Genome. 2003 September; 14(9):657-63 and Liefers et al., J Dairy Sci. 2003 March; 86(3):799-807) show that cows with higher dry matter intake were significantly heavier and had significantly higher serum leptin concentration. In addition, these authors also showed that cows with a negative energy balance (strongly related to lower body weight and lower body condition) had significantly lower serum leptin concentration compared to positive energy balance cows. In the present data serum leptin concentration is positively related to feed intake (r=0.26) and body weight (r=0.25), thus confirming the findings by Liefers et al. (2003). It has been observed in mice that obviously obese mice with higher serum leptin still continued to eat more (Houseknecht et al., J Anim Sci. 1998 May; 76(5): 1405-20). Evidence in the literature shows that response to the inhibitory feedback effects of leptin is more sensitive in leaner animals, and sensitivity is greatly reduced in animals with large fat stores (cattle generally have higher body fat content reminiscent of obesity in other species), even though circulating concentrations of leptin in the latter group are high (Houseknecht et al., J Anim Sci. 1998 May; 76(5):1405-20). Perhaps, the findings of this study may form the basis of leptin resistance in cattle. This phenomenon of leptin-resistance in certain obese individuals is not-yet clearly understood, though it has been suggested that some of the leptin receptor forms may be involved in incidence of leptin resistance (Houseknecht et al., J Anim Sci. 1998 May; 76(5):1405-20).

Average backfat thickness (additive effect, a=−2.29±0.50 mm); final backfat thickness (additive effect, a=−4.31±0.95 mm); and ultrasound backfat thickness were significantly higher (P<0.001) for animals with the T allele of UASMS2 than for animals with the C allele. Similarly, the T allele of UASMS2 was significantly associated with higher (P<0.01) average ultrasound marbling score (additive effect, a=−0.61±0.21) and final marbling score (additive effect, a=−0.89±0.25, P<0.01) compared to the C allele. These results are not surprising as the correlation between ultrasound marbling and backfat thickness in the present data set was also high (r=0.54) (data not shown). Taken to a constant body weight of 500 kg through linear regression predictions, animals with the TT genotype of UASMS2 had significantly higher ultrasound backfat (P<0.001) and marbling scores (P<0.01) compared to animals with the CC genotypes. The significant increases in body fatness in animals with the T allele of UASMS2 was associated with slight reductions (P<0.05) in final (additive effect, a=5.60±2.50 cm²,) and average (additive effect, a=4.03±1.58 cm²) longissimus thoracis area. Measures of carcass weight and body fat were generally higher in animals with the T allele compared to the C allele. However, there were only a few animals with the TT genotype that had carcass data for comparison and thus there were no statistical differences among genotypes of UASMS2 in these carcass traits. The opposite is true with carcass measures of lean meat yield and longissimus muscle area. Categorical data analysis of the carcass grades (A, AA, and AAA) among genotypes of UASMS2 showed no significant associations between quality grade and genotypes (χ²=1.14, P=0.56) (Table 11).

Residual feed intake tended to differ (P<0.10) among UASMS2 genotypes and was lower in CT (dominance effect, d=0.42±0.21 kg d⁻¹) than in the homozygotes. Feed conversion ratio and partial efficiency of growth did not differ (P>0.30) among genotypes of UASMS2. The present data also did not show statistical significance in final weight, mean body weight, slaughter weight and carcass weight among animals with different UASMS2 genotypes (obviously due to the very few TT animals available for comparison and associated with high standard errors of genotype means). However, the T allele was generally associated with higher body weights with differences between TT and CC animals in mean body weight, final weight and slaughter weight of 30.34 kg, 42.02 kg and 36.37 kg, respectively. Feeding duration (dominance effect, d=5.07±2.61 min d⁻¹) and feeding head down time (dominance effect, d=5.12±2.51 min d⁻¹) differed among genotypes and were higher in heterozygotes of UASMS2 than homozygotes (P<0.05). Feeding frequency tended to differ among genotypes (P<0.10) among genotypes of UASMS2 and was higher for CC animals than for TT animals (additive effect, a=4.47±2.86 events d⁻¹).

### Example 6

### Associations of EXON2-FB with Various Phenotypic Traits

The effect of different genotypes of EXON2-FB on measures of serum leptin concentration, performance, feed efficiency, feeding behavior and ultrasound and carcass merit are presented in Tables 9 and 10. Metabolic midpoint weight was lower (P<0.05) for animals with genotype TT than for CC (additive effect, a=4.16±1.61 kg^{0.75}). Average daily gain tended to differ among genotypes (P<0.10) and was lower in TT animals compared to CC animals (additive effect, a=0.12±0.05 kg d⁻¹). Average backfat thickness (additive effect, a=−0.56±0.19 mm) and final ultrasound backfat (additive effect, a=−1.07±0.17 mm) were lower (P<0.05) for animals with genotype CC than for TT (Buchanan et al., Genet Sel Evol. 2002 January-February; 34(1):105-16). Feeding duration tended to differ (P=0.08) among genotypes of EXON2-FB and was higher for CC animals than for CT animals (dominance deviation, a=−2.71±1.63 events d⁻¹). Feeding frequency was different (P=0.01) among genotypes of EXON2-FB and was higher for TT animals than for CT animals (dominance deviation, a=−2.66±1.11 events d⁻¹) or CC animals (additive effect, a=−3.30±1.51 events d⁻¹).

TABLE 9
Effect of different genotypes of EXON2-FB (least-squares
means ± standard error) on measures of serum leptin,
performance, efficiency and feeding behavior of hybrid cattle
EXON2-FB Marker genotype^{z}

| Trait | CC | CT | TT | P value^{y} |
|---|---|---|---|---|
| Number of animals | 50 | 68 | 32 | |
| Serum leptin, performance | | | | |
| and efficiency | | | | |
| Serum leptin level, ng ml⁻¹ | 13.69 ± 1.13 | 12.86 ± 0.99 | 13.02 ± 1.43 | 0.78 |
| Metabolic mid-weight, kg^{.75} | 88.93 ± 1.24 | 86.17 ± 1.07 | 84.77 ± 1.57 | 0.02 |
| Average daily gain, kg d⁻¹ | 1.43 ± 0.04 | 1.36 ± 0.04 | 1.32 ± 0.05 | 0.07 |
| Residual feed intake, kg d⁻¹ | −0.44 ± 0.24 | −0.63 ± 0.24 | −0.61 ± 0.27 | 0.40 |
| Feed conversion ratio | 6.07 ± 0.14 | 6.01 ± 0.12 | 6.08 ± 0.18 | 0.89 |
| Dry matter intake, kg d⁻¹ | 7.73 ± 0.53 | 7.51 ± 0.53 | 7.45 ± 0.54 | 0.21 |
| ME intake, KJ kg^{0.75} d⁻¹ | 1069.3 ± 62.7 | 1041.2 ± 62.9 | 1035.5 ± 64.4 | 0.22 |
| Partial Efficiency of growth, | 0.33 ± 0.02 | 0.34 ± 0.02 | 0.33 ± 0.02 | 0.50 |
| Feeding Behavior | | | | |
| Feeding duration, min d⁻¹ | 56.19 ± 7.40 | 52.05 ± 7.46 | 52.96 ± 7.58 | 0.08 |
| Head down time, min d⁻¹ | 36.44 ± 3.24 | 33.19 ± 3.09 | 33.55 ± 3.35 | 0.18 |
| Feeding frequency, events d⁻¹ | 32.04 ± 1.88 | 31.03 ± 1.81 | 35.34 ± 2.08 | 0.01 |

^{z}EXON2-FB polymorphism is a C/T substitution located at position 305 of exon 2 of the bovine leptin gene according to SEQ ID NO: 5 (Gen bank accession no. AY138588 - Buchanan et al., 2002).
^{y}P value = probability of differences among different marker genotypes

TABLE 10
Effect of different genotypes of EXON2-FB (least-squares means ± standard error)
on measures of body weight, ultrasound and carcass merit of hybrid cattle
EXON2-FB Marker genotype^{z}

| Trait | CC | CT | TT | P value^{y} |
|---|---|---|---|---|
| Number of animals | 50 | 68 | 32 | |
| Body weight and ultrasound data | | | | |
| Initial Measurements (Jan 10) | | | | |
| Body weight, kg | 353.99 ± 6.68 | 341.70 ± 5.84 | 337.74 ± 8.47 | 0.12 |
| Ultrasound backfat, mm | 4.37 ± 0.27 | 4.72 ± 0.23 | 5.00 ± 0.34 | 0.17 |
| Ultrasound marbling score | 4.36 ± 0.03 | 4.35 ± 0.03 | 4.31 ± 0.04 | 0.47 |
| Longissimus thoracis area, cm² | 61.90 ± 0.37 | 61.72 ± 0.32 | 61.69 ± 0.46 | 0.91 |
| Final Measurements (May 01) | | | | |
| Body weight, kg | 510.43 ± 8.73 | 489.68 ± 7.63 | 480.11 ± 11.07 | 0.02 |
| Ultrasound backfat | 6.40 ± 0.15 | 6.90 ± 0.13 | 7.47 ± 0.19 | 0.03 |
| Ultrasound marbling score | 4.98 ± 0.09 | 4.96 ± 0.07 | 5.10 ± 0.08 | 0.52 |
| Longissimus thoracis area, cm² | 74.30 ± 1.11 | 73.07 ± 0.97 | 72.71 ± 1.41 | 0.46 |
| Average measurements^{x} | | | | |
| Body weight, kg | 416.9 ± 2.44 | 415.04 ± 2.31 | 413.92 ± 2.49 | 0.28 |
| Ultrasound backfat | 4.82 ± 0.16 | 5.05 ± 0.14 | 5.38 ± 0.21 | 0.04 |
| Ultrasound marbling score | 4.34 ± 0.06 | 4.35 ± 0.06 | 4.35 ± 0.07 | 0.94 |
| Longissimus thoracis area, cm² | 68.01 ± 0.46 | 68.10 ± 0.40 | 68.46 ± 0.58 | 0.74 |
| Predicted @ 500 kg | | | | |
| Ultrasound backfat | 6.54 ± 0.31 | 6.47 ± 0.26 | 7.22 ± 0.38 | 0.19 |
| Ultrasound marbling score | 4.89 ± 0.08 | 4.87 ± 0.07 | 5.05 ± 0.10 | 0.33 |
| Longissimus thoracis area, cm² | 71.49 ± 0.79 | 71.61 ± 0.68 | 71.41 ± 0.99 | 0.98 |
| Carcass data | | | | |
| Number of animals | 36 | 47 | 26 | |
| Slaughter weight, kg | 510.23 ± 8.85 | 489.62 ± 7.67 | 479.87 ± 11.21 | 0.02 |
| Carcass weight, kg | 306.99 ± 5.28 | 286.81 ± 4.63 | 287.17 ± 6.27 | 0.01 |
| Grade Fat, mm | 8.98 ± 0.56 | 8.19 ± 0.48 | 9.55 ± 0.65 | 0.23 |
| Ave. Backfat, mm | 10.51 ± 0.58 | 9.72 ± 0.50 | 10.99 ± 0.68 | 0.29 |
| Carcass marbling score | 2.37 ± 0.09 | 2.21 ± 0.08 | 2.44 ± 0.11 | 0.20 |
| L. thoracis area, cm² | 76.12 ± 2.73 | 75.18 ± 2.60 | 74.63 ± 2.54 | 0.67 |
| Lean meat yield, % | 58.07 ± 0.54 | 58.76 ± 0.46 | 57.63 ± 0.63 | 0.32 |

^{z}EXON2-FB polymorphism is a C/T substitution located at position 305 of exon 2 of the bovine leptin gene according to SEQ ID NO: 5 (Gen bank accession no. AY138588 - see also Buchanan et al., 2002).
^{y}P value = probability of differences among different marker genotypes.
^{x}Average of five measurements taken between January 10 and May 01 at approximately monthly intervals

Final body weight (additive effect, a=30.32±9.9 kg) and carcass weight (additive effect, a=19.82±5.78 kg), P=0.01) were lower (P<0.05) for TT animals of EXON2-FB compared to the CC animals. No significant associations were detected between EXON2-FB and the other traits studied. Measures of carcass fatness were generally higher and measures of carcass lean meat yield and longissimus muscle area were lower for TT animals compared to CC animals of EXON2-FB, though no statistical significance was detected. Chi-square analysis of the carcass grades (A, AA, and AAA) among genotypes of EXON2-FB showed no significant associations between quality grade and genotypes (X²=0.95, P=0.62) (Table 11).

Three polymorphisms in the bovine leptin promoter are associated with growth rate, body weight, feed intake, feeding behavior and ultrasound merit. Though some differences in carcass fatness were detected, these were not statistically significant, possibly due to the removal of some extreme animals based on residual feed intake (correlation between RFI and backfat is about r=0.25) for some metabolic studies. In addition, one of the markers, UASMS2 is associated with serum leptin levels in cattle. The frequency of this SNP was very low in both the experimental population and the five commercial lines of cattle studied.

TABLE 11
Distribution of carcass quality grades among
genotypes of the different markers

| | | Carcass quality grades | | | |
|---|---|---|---|---|---|
| Polymorphism | Genotype | A | AA | AAA | Chi-square test |
| | CC-CC | 5 | 10 | 7 | |
| UASMS1-3 | CT-CG | 13 | 27 | 9 | χ² = 1.37, P = 0.50 |
| | TT-GG | 7 | 23 | 8 | |
| | CC | 20 | 40 | 16 | |
| UASMS2 | CT | 4 | 18 | 7 | χ² = 1.14, P = 0.56 |
| | TT | 1 | 2 | 1 | |
| | CC | 6 | 22 | 8 | |
| EXON2-FB | CT | 14 | 25 | 8 | χ² = 0.95, P = 0.62 |
| | TT | 5 | 13 | 8 | |

Unlike the UASMS1 and UASMS3 polymorphisms, the UASMS2 and UASMS3 polymorphisms are not linked. This can be seen in Table 12 which illustrates the linkage disequilibrium between the UASMS2 and UASMS3 polymorphisms.

TABLE 12
Test of linkage disequilibrium using percentage
deviations of observed from expected pairwise
genotype combinations of UASMS3 and UASMS2

| | | UASMS3 genotypes | | |
|---|---|---|---|---|
| | | CC | CG | GG |
| UASMS2 genotypes | Frequency | 0.18 | 0.46 | 0.36 |
| CC | 0.63 | 6.96 | −0.68 | −6.58 |
| CT | 0.32 | −5.76 | 2.58 | 3.58 |
| TT | 0.05 | −2.30 | −0.90 | 3.20 |

### Example 7

### Association of Multiple SNPs in the Leptin Gene with Carcass and Meat Quality Traits in Beef Cattle

Five SNPs (UASMS1, UASMS2, UASMS3, E2JW and EXON2-FB) were genotyped on 1,111 crossbred bulls, heifers and steers. The measured traits included fat, lean and bone yield (%) by partial rib dissection, grade fat, longissimus muscle (LM) area, hot carcass weight, quality grade, LM intramuscular fat, and tenderness evaluation of LM and semitendinosus muscle. Only four SNPs were analyzed (UASMS1, UASMS2, E2JW and EXON2-FB) because UASMS1 and UASMS3 were completely linked. A univariate mixed inheritance animal model was used to evaluate the association of the SNP genotypes or haplotypes with the traits. The two leptin exon 2 SNPs were associated with fat and lean yield and grade fat (E2JW, P<0.01; EXON2-FB, P<0.05) and they interacted in their effect on LM tenderness (P<0.01). The leptin promoter SNP were either not associated with any of the traits (UASMS2) or with fat yield only (UASMS1). Three haplotypes (TCAC, CCAT, TTAC) were at high frequency in the population (88%) and had similar effects on all the traits. Compared to the common haplotypes, one haplotype (CCTT) showed a significantly different effect on FATYL, GFAT and LEANYL (P<0.01) and one haplotype (TTTT) on LM tenderness (P<0.03). Therefore, important associations between single nucleotide polymorphisms within the leptin gene with lean yield and tenderness were detected. Cattle: The animals were commercially fed heifers (165), steers (231) and bulls (61) from industry sires, heifers (40), steers (375), and bulls (48) from the University of Guelph breeding project and steers from a University of Guelph feeding trial carried out at a feedlot in Rockwood, Ontario. The three sources of cattle were identified as Commercial, Elora and Rockwood, respectively. Animals were crossbred with breed composition formed by several breeds. The major contributing breeds were Angus (AN), Charolais (CH), Limousin (LM), and Simmental (SM). The average contribution of these four breeds to the breed composition of animals having any fraction of the mentioned breeds were 0.46, 0.50, 0.50, and 0.50 for AN, CH, LM, and SM, respectively, for Commercial cattle; 0.24, 0.36, 0.38, and 0.41 for Elora cattle; and 0.51, 0.53, 0.59, and 0.41 for Rockwood cattle.

DNA isolation, polymorphisms detection and genotyping: EXON2-FB (Buchanan et al., Genet Sel Evol. 2002 January-February; 34(1):105-16) and E2JW (Lagonigro et al., Anim Genet. 2003 October; 34(5):371-4, originally referred to as 252-SNP) were within exon 2 of the ob gene. The genotyping of each SNP was carried out using the 5′ nuclease allelic discrimination assay on an ABI PRISM™ 7700 sequence detector (Applied Biosystems Inc.). Details of procedures were described by Nkrumah et al. (J Anim Sci. 2005 January; 83(1):20-8) incorporated herein by reference in its entirety. The DNA from a subset of the genotyped animals was sequenced across each polymorphism and the sequence results were used to confirm the genotypes obtained by discrimination assays.

Phenotypic information: Information on tenderness of Longissimus muscle (shear force) at 2 (LM2), 7 (LM7), 14 (LM14) and 21 (LM21) days postmortem and of Semitendinosus muscle at 7 (ST7) days postmortem, chemical fat (CF), grade fat (GFAT), quality grade (QG), Longissimus muscle area (LMA), lean (LEANYL), fat (FATYL) and bone (BONEYL) yield and hot carcass weight (HCW) were available on most of the 1,111 genotyped animals as shown in Table 13 below.

TABLE 13
Number of phenotypic records on the carcass
and meat quality traits with corresponding means,
SD, and coefficient of variation (CV)

| Traits^{a} | Records | Mean | SD | CV (%) |
|---|---|---|---|---|
| FATYL (%) | 905 | 24.5 | 5.12 | 20.9 |
| LEANYL (%) | 905 | 56.1 | 5.03 | 9.0 |
| BONEYL (%) | 905 | 19.4 | 2.62 | 13.5 |
| GFAT (mm) | 914 | 9.3 | 3.37 | 36.2 |
| LMA (cm²) | 892 | 86.7 | 13.64 | 15.7 |
| HCW (kg) | 911 | 336.0 | 49.23 | 14.7 |
| LM2 (kg) | 711 | 5.3 | 1.71 | 32.3 |
| LM7 (kg) | 876 | 4.8 | 1.40 | 29.2 |
| LM14 (kg) | 875 | 4.3 | 1.25 | 29.1 |
| LM21 (kg) | 869 | 3.8 | 0.96 | 25.3 |
| LMAVG (kg) | 707 | 4.5 | 1.03 | 22.9 |
| ST7 (kg) | 869 | 5.3 | 1.10 | 20.7 |
| CF (%) | 920 | 4.0 | 1.58 | 39.5 |

| | | |
|---|---|---|
| QG^{b} | 915 | A - 15.4% AAA - 25.5% AA - 59.1% |

^{a}Lean (LEANYL), fat (FATYL) and bone (BONEYL) yield, grade fat (GFAT), Longissimus muscle area (LMA), hot carcass weight (HCW), Longissimus muscle shear force (LM) at 2, 7, 14 and 21 days postmortem and average shear force across aging times (LMAVG), semitendinosus muscle shear force (ST) at 7 days postmortem, longissimus muscle chemical fat (CF), and quality grade (QG).
^{b}Observed frequency of the quality grades.

Warner-Bratzler shear force measurements (kg) were used as an objective method of assessing tenderness (Shackelford et al., 1999). Shear force is the physical test done on a cooked meat core sample that determines the force (in kg) necessary to separate the muscle fibers. Grade fat is the backfat thickness measurement taken at the 12^{th} and 13^{th} rib interface. Longissimus muscle area is the measure of the Longissimus dorsi muscle area at the 12^{th} and 13^{th} rib interface using a tracing of the muscle. Chemical fat is the chemical analysis on a core meat sample that determines the percent intra-muscular fat. Lean, fat, and bone yield were determined by dissection of a 4-bone rib section. Quality grade is the marbling grade used for grading in Canada with most carcasses falling in one of three grades (A, AA, AAA). Because only few carcasses were classified as Prime, those animals were combined with AAA carcasses for the analyses.

Results: A total of 1,104, 1,111, 1,106, 1,068 and 1,109 animals had genotypes available for UASMS1, UASMS2, UASMS3, EXON2-FB and E2JW SNPs, respectively. The genotypes for UASMS1 and UASMS3 were almost perfectly linked. Only three out of 1,104 genotypes for UASMS1 and UASMS3 did not mach each other (that is, the C and T alleles in UASMS1 were not associated with the C and G alleles in UASMS3, respectively, in only three animals). Thus, UASM3S was dropped from the analyses and allele frequencies and association with the traits for UASMS1 were extended to UASMS3. The genotypes of all animals were used to determine the allelic frequencies. For the study of association between SNPs in the ob gene and carcass and meat quality traits, only animals with required phenotypic information and with genotypes available for all four SNPs (UASMS1, UASMS2, E2JW and EXON2-FB) were used. The resulting number of records ranged from 711 for LM2 to 920 for CF. Table 13 gives the number of records, mean, SD, and coefficient of variation of the analyzed traits.

Statistical analyses: All analyses were performed using the statistical software SAS (SAS Institute Inc.,) and ASREML (Gilmour et al.,). Descriptive characteristics of quantitative traits were obtained using SAS PROC MEANS. Allele frequencies were tabulated and compared by Chi-square analysis using SAS PROC FREQ.

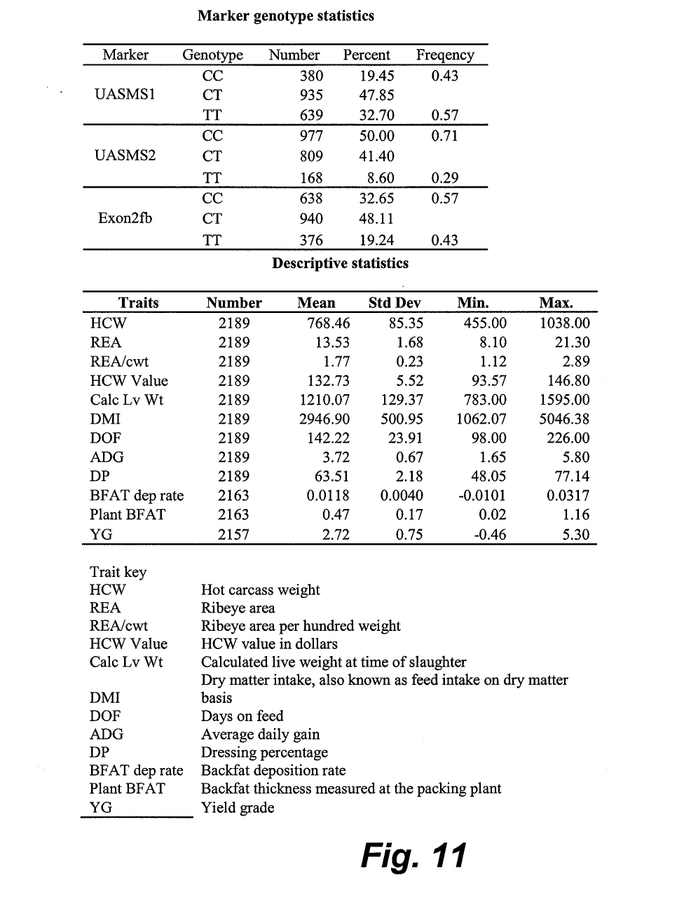

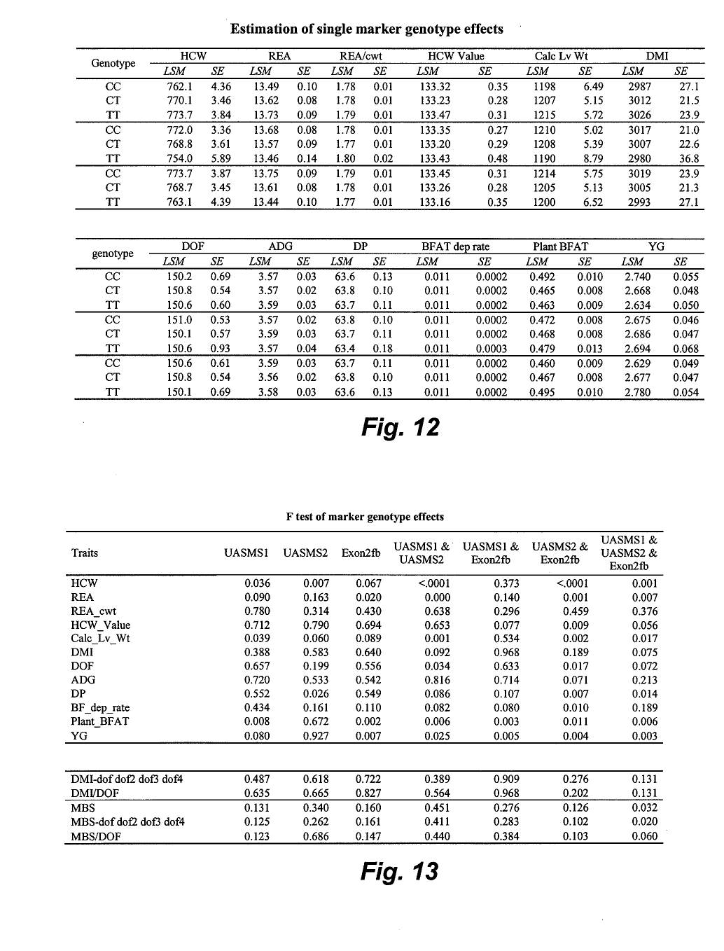

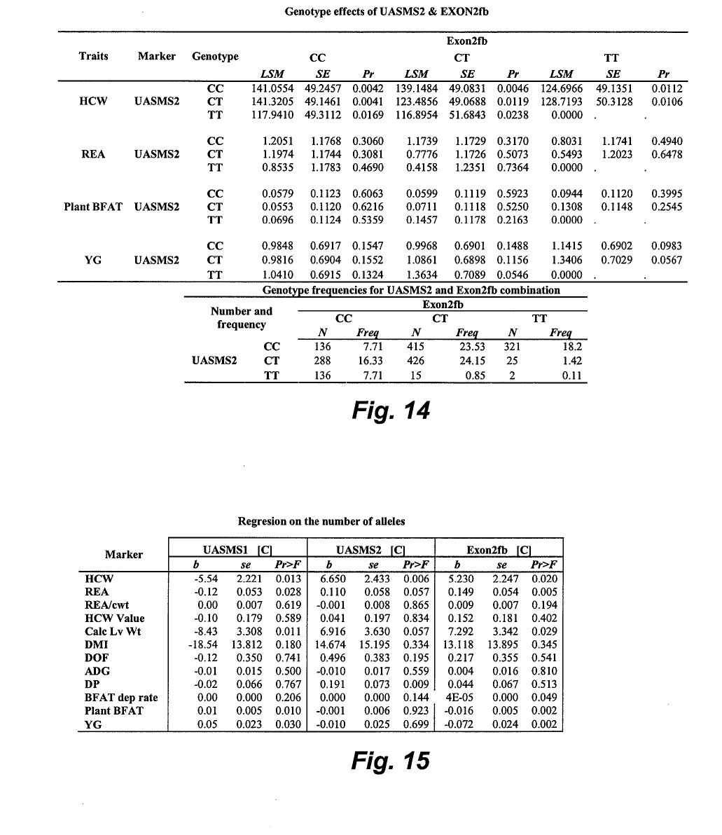

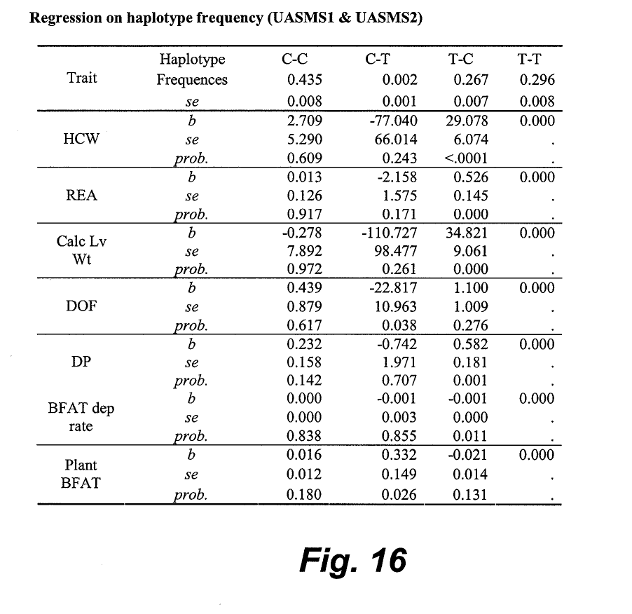

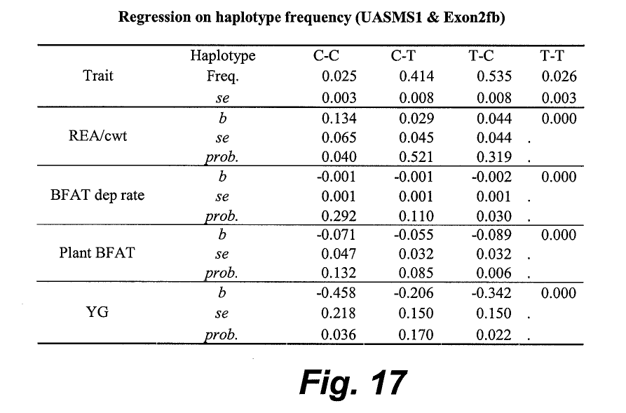

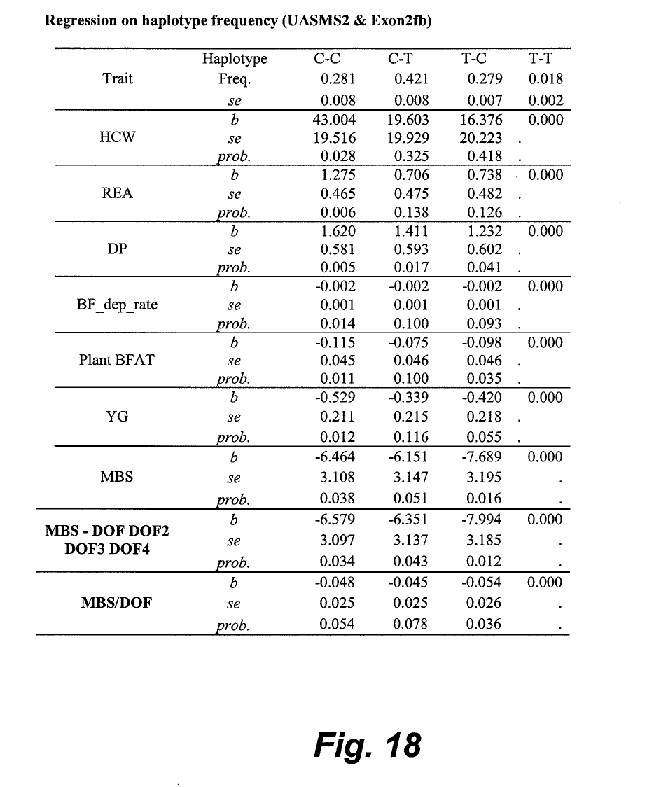

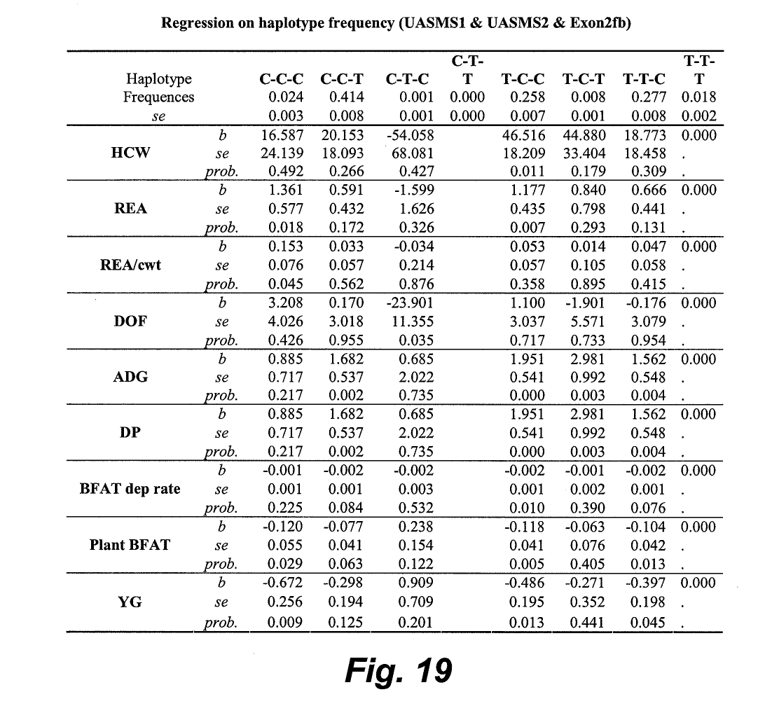

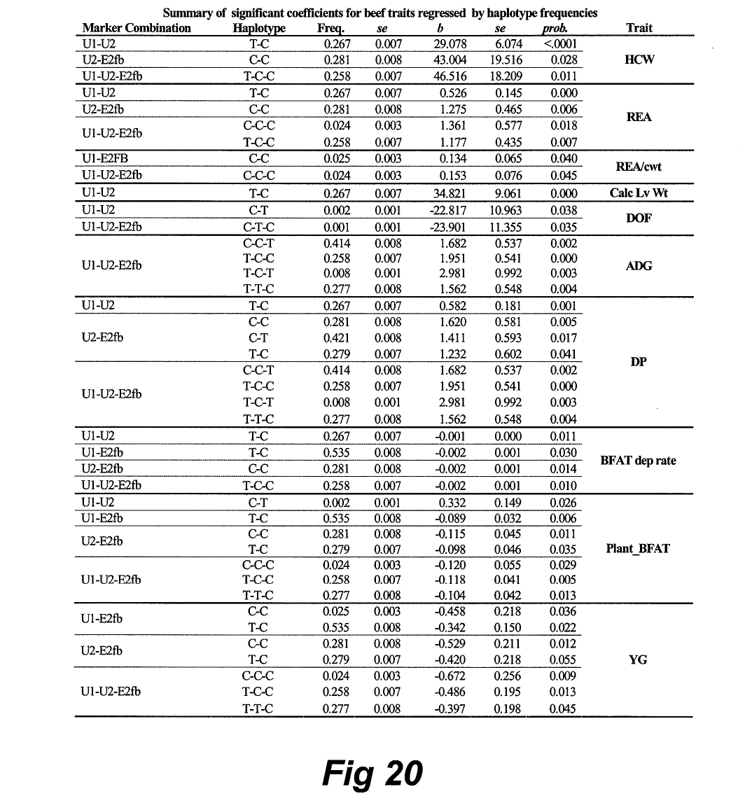

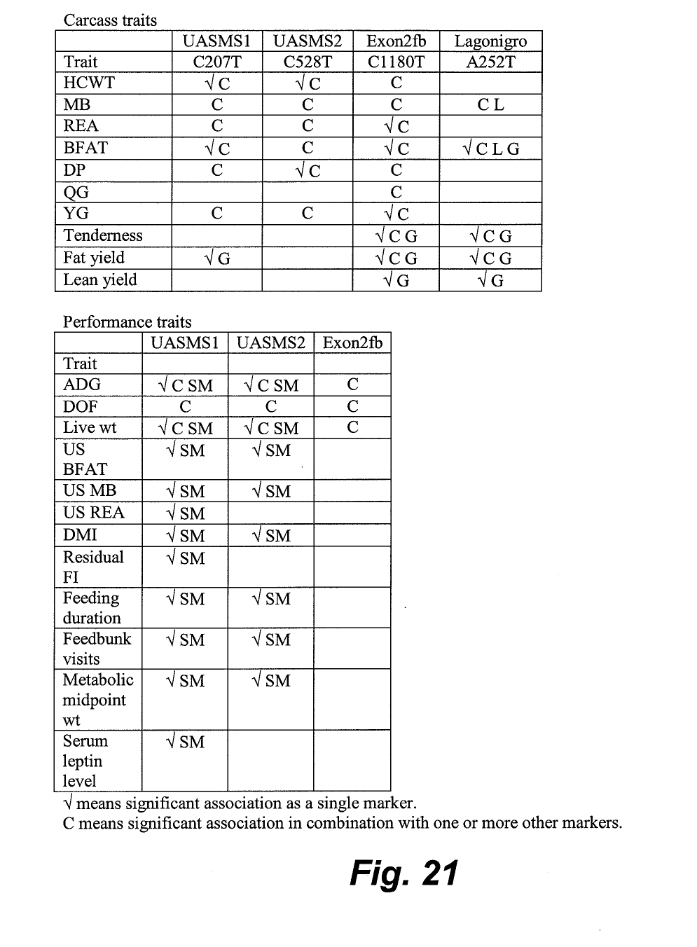

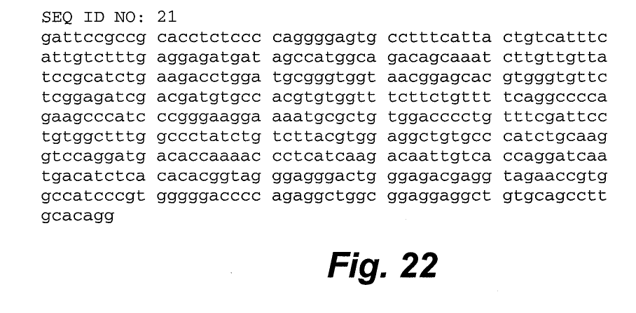

In a second batch of test animals, associations between combinations of genotypes were studied, the data being shown in FIGS. 11-20. Associations of single markers and traits in the test cattle are summarized in FIG. 21.

### Example 8

### Genotype Analyses

Association of the genotypes with the traits was evaluated by genetic analysis using ASREML, fitting a mixed inheritance model (SNP genotypes plus polygenic effects). The model included SNP genotypes as fixed effects:
Yᵢⱼₖₗₘ=u+Σ⁴ⱼ₌₁₎Genᵢ₍ⱼ₎+Sexₖ+Slg₁+β₁AN+β₂LM+β₃CH+β₄SM+POlₘ+eᵢⱼₖₗₘ (1)
Where: Yᵢⱼₖₗₘ is the trait measured in the m-th animal of k-th sex and l-th slaughter group; u is the overall mean for the trait; Genᵢ₍ⱼ₎ is the effect of the i-th genotype for j-th SNP (UASMS1, UASMS2, E2JW, and EXON2-FB) in the leptin gene; Sexₖ is the fixed effect of the k-th sex (bull, heifer and steer); Slg₁ is the fixed effect of the l-th slaughter group (94 levels); β₁, β₂, β₃, β₄ are the regression coefficients on breed composition of AN, CH, LM, and SM, respectively; Polₘ is the random additive genetic (polygenic) effect of the m-th animal; eᵢⱼₖₗₘ is the residual random effect associated with the m-th animal.

Following Fernando at al. (J Dairy Sci. 1998 September; 81 Suppl 2:64-75), as the genotypes were known, the mixed model equations of CR Henderson for model (1) were used in the analyses. The additive relationship matrix based on the general pedigree was used for modeling the covariances among polygenic effects. Animals originated from 125 sires and all sires were known. With respect to the dams, 43% of the animals had dams identified. Average size of paternal half-sib families was 8.9. Percentages of sires with less than 5, from to, from 6 to 10 and more than 15 offspring were 36%, 26.4%, 27.2% and 10.4%, respectively.

Slaughter groups were defined as animals from the same source (Commercial or Rockwood) and with the same slaughter date or animals from Elora coming from the same trial and feed treatment, and killed in the same season (December-February, March-May, June-August, and September-November).

The repeated shear force measurements of LM across postmortem periods were analyzed individually within each period, as the average shear force over periods (LMAVG), and as the intercept and slope of the individual linear regression of shear force measurements on postmortem days. The effect of the four SNP in the Leptin gene on quality grade was analyzed by chi-square analysis (PROC FREQ), as well as a linear trait using ASREML, applying model (1). In this case, scores of 1, 2, and 3 were assigned to quality grades A, AA and AAA, respectively.

To keep reasonable probability values for Type I error, two levels of tests were performed. For initial assessment of the results, an overall value of P<0.05 (α) was used. For a more detailed review of the results, a modified Bonferroni correction was used (a/√n, Mantel, Arch Toxicol Suppl. 1980; 3:305-10, Mantel, Biometrics. 1980 September; 36(3):381-99) to account for the number of tests. The value of n was determined using a SNP-wise approach combined with grouping traits according to type (Ye et al., Yi Chuan. 2003 January; 25(1):89-92). Traits were grouped into two groups as follows: carcass yield traits (LEANYL, FATYL, BONEYL, GFAT, LMA, and HCW) and meat quality traits (CF, QG, LM, LM, LM, LM, LMAVG, and ST). Because there were four SNPs, n was equal to 24 (4×6) and 32 (4×8) for carcass and meat quality traits, respectively, with the corresponding modified Bonferroni corrected significance levels of 0.010 and 0.009.

Initially two-way interactions between SNP were fit into the model, but there was only a significant interaction between E2JW and EXON2-FB SNP for shear force of LM.

For all other traits, the interactions were dropped from the model. For LM shear force, the joint genotype effect of E2JW and EXON2-FB were included in the model.

Variances were estimated from the data and assumed known for estimation and testing purposes. Probabilities associated with the Wald F statistics output by ASREML were obtained using error degrees of freedom that account for the estimated fixed effects, but ignore the fact variances were estimated. This, however, should not be a problem, because the number of records on all traits was relatively high and variances were estimated by translation invariant functions of the data by REML.

### Example 9

### Haplotype Analyses

Association of the haplotypes for the SNP in the Leptin gene and carcass and meat quality traits was evaluated by genetic analysis using ASREML, applying model (1) replacing genotype effects by regressions on haplotype probabilities. The haplotype probabilities were reconstructed using the algorithm and software (HAPROB) (Boettcher et al., J Dairy Sci. 2004 December; 87(12):4303-10). This software estimated probabilities of haplotype combinations for members of half-sib families, given that genotypes are known for all siblings, but unknown for all parents. The accuracy of reconstruction of the halfsibs' haplotypes by the HAPROB software is considerably high. For instance, the accuracy varies from 64% to 94% for reconstruction of haplotypes of individuals from halfsib families of size from 2 to 10 offspring, when three loci with three alleles are considered (Boettcher et al., J Dairy Sci. 2004 December; 87(12):4303-10). Table 14 shows the possible sixteen haplotypes with their corresponding probabilities.

TABLE 14
Haplotypes probabilities in the beef population

| Haplotypes | | | | | | |
|---|---|---|---|---|---|---|
| | | | EXON2- | | | |
| UASMS1 | UASMS2 | E2JW | FB | Prob 1^{a} | Prob 2 | Code^{b} |
| T | C | A | C | 0.34241 | 0.35177 | 1 |
| C | C | A | T | 0.33621 | 0.33133 | 2 |
| T | T | A | C | 0.20399 | 0.20407 | 3 |
| C | C | A | C | 0.02217 | 0.02116 | 4 |
| T | C | A | T | 0.02037 | 0.02084 | 5 |
| T | T | A | T | 0.01862 | 0.01532 | 6 |
| C | C | T | T | 0.01757 | 0.01717 | 7 |
| T | T | T | T | 0.01619 | 0.01215 | 8 |
| C | T | A | T | 0.01550 | 0.01465 | 9 |
| C | T | A | C | 0.00379 | 0.00362 | 10 |
| T | C | T | T | 0.00272 | 0.00212 | 10 |
| T | C | T | C | 0.00201 | 0.00237 | 10 |
| T | T | T | C | 0.00166 | 0.00166 | 10 |
| C | T | T | T | 0.00098 | 0.00104 | 10 |
| C | C | T | C | 0.00038 | 0.00031 | 10 |
| C | T | T | C | 0.00036 | 0.00047 | 10 |

^{a}Prob 1 = Probability of the haplotype in all genotyped animals; Prob 2 = Probability of the haplotype in animals genotyped for all four SNP and with phenotypic records.
^{b}Haplotype code for the analyses.

The three most frequent haplotypes had a summed probability of 0.88. Therefore, there were many rare haplotypes and some of them were joined into one group containing the least probable haplotypes, which were referred to as haplotype 10.

### Example 10

### Allele Frequencies

The Chi-square test for differences in allele frequency among breeds (animals with breed composition≧⅝ for a given breed) was not significant for any SNP in the Leptin gene as shown in Table 15.

TABLE 15
Allele frequencies (%) within breeds and in the entire beef population
for the UASMS1, UASMS2, E2JW and EXON2-FB SNP in the Leptin gene
Breed^{a}

| SNP^{b} | Alleles | Angus | Limousin | Charolais | Simmental | Other | Total |
|---|---|---|---|---|---|---|---|
| UASMS1 | C | 48.8 | 48.3 | 45.4 | 34.6 | 38.4 | 38.9 |
| | T | 51.2 | 51.7 | 54.6 | 65.4 | 61.6 | 61.1 |
| | N^{c} | 43 | 30 | 11 | 68 | 952 | 1,104 |
| UASMS2 | C | 73.3 | 65.5 | 77.3 | 69.8 | 74.4 | 73.8 |
| | T | 26.7 | 34.5 | 22.7 | 30.2 | 25.6 | 26.1 |
| | N | 43 | 29 | 11 | 68 | 960 | 1,111 |
| EXON2-FB | C | 45.4 | 51.7 | 54.6 | 58.8 | 58.3 | 57.6 |
| | T | 54.6 | 48.3 | 45.4 | 41.2 | 41.7 | 42.4 |
| | N | 43 | 30 | 11 | 68 | 916 | 1,068 |
| E2JW | A | 95.4 | 95.0 | 90.9 | 97.8 | 96.0 | 96.0 |
| | T | 4.6 | 5.0 | 9.1 | 2.2 | 4.0 | 4.0 |
| | N | 43 | 30 | 11 | 68 | 957 | 1,109 |

^{a}Animals with breed composition ≦⅝ for a given breed. Other includes animals with breed composition ≦⅝ for all breeds.
^{b}No significant differences in allele frequencies among breeds (P = 0.11, P = 0.46, P = 0.15, and P = 0.47 by Chi-square test for UASMS1, UASMS2, EXON2-FB, and E2JW, respectively).
^{c}Number of animals.

However, for UASMS1, Simmental tended to have lower frequency of the C allele than the other breeds (P=0.11) and, for EXON2-FB, Angus tended to have lower frequency of C allele than the other breeds (P=0.15).

The T allele was predominant over the C allele for UASMS1 and EXON2-FB (57.6% vs. 42.4% and 61.1% vs. 38.9%, respectively), while for UASMS2 the C allele was much more common than T (73.8% vs. 26.1%). The E2JW SNP showed the largest difference in allele frequencies in the population. For this SNP, the T allele was rare compared to the A allele (4.0% vs. 96.0%).

The frequencies of genotypes were in agreement with Hardy-Weinberg equilibrium (Falconer and Mackay, 1996) within all SNP (the probabilities of the Chi-Square tests for deviation from the equilibrium were equal to 0.745, 0.169, 0.975, and 0.995 for UASMS1, UASMS2, E2JW and EXON2-FB, respectively).

Equilibrium in genotypic frequencies when considering jointly two SNP was tested by a ChiSquare test of expected and observed frequencies of gametic types (Falconer and Mackay, 1996). The only two SNP whose genotypes were jointly in equilibrium were UASMS1 and E2JW. All other pairwise tests showed a significant disequilibrium (P<0.01).

### Example 11

### Genotype Analyses

Genotypes did not significantly influence LMA, BONEYL, CF, HCW, ST7 and QG as shown in Table 16.

TABLE 16
Test for the association of SNPs in Leptin gene with lean
(LEANYL), fat (FATYL) and bone (BONEYL) yield, grade fat
(GFAT), chemical fat (CF), Longissimus muscle area (LMA),
hot carcass weight (HCW), Longissimus muscle shear force
(LM) at 2, 7, 14 and 21 days postmortem and average shear
force (LMAVG), semitendinosus muscle shear force (ST) at 7
days postmortem, and quality grade (QG) in the beef cattle population.
P > F^{a}
SNP in Leptin gene

| Trait | UASMS1 | UASMS2 | E2JW | | EXON2-FB |
|---|---|---|---|---|---|
| LEANYL | 0.110 | 0.278 | 0.003 | | 0.038 |
| FATYL | 0.012 | 0.387 | 0.010 | | 0.013 |
| BONEYL | 0.664 | 0.101 | 0.277 | | 1.000 |
| GFAT | 0.081 | 0.449 | 0.006 | | 0.016 |
| LMA | 0.771 | 0.110 | 0.216 | | 0.795 |
| HCW | 0.691 | 0.625 | 0.764 | | 0.787 |
| CF | 0.861 | 0.932 | 0.603 | | 0.712 |
| LM2 | 0.779 | 0.819 | | 0.005b | |
| LM7 | 0.403 | 0.795 | | 0.054 | |
| LM14 | 0.098 | 0.364 | | 0.009 | |
| LM21 | 0.733 | 0.353 | | 0.085 | |
| LMAVG | 0.566 | 0.419 | | 0.001 | |
| ST7 | 0.887 | 0.0 | 0.111 | 0.502 | |
| QG | 0.492 | 0.970 | 0.777 | | 0.619 |
| QG Chi-Square | 0.141 | 0.734 | 0.300 | | 0.472 |

^{a}Significance level of the Walt F-test for the effect of genotypes on carcass and meat quality traits. For QG, a Chi-Square test for the effects of genotypes was also performed.
bFor tenderness of longissimus muscle, a significant interaction between E2JW and EXON2-FB was found. The genotypes for these two SNP were analyzed jointly.

Genotypes for E2JW and EXON2-FB significantly influenced LEANYL, FATYL, and GFAT, while genotypes for UASMS1 (or, alternatively, UASMS3) significantly influenced FATYL. The analyses of LM shear force in each particular postmortem day and as an average shear force over the postmortem days showed a significant effect of the E2JW.EXON2-FB genotypes (the interaction E2JW by EXON2-FB was significant (Table 16)).

Table 17 presents the least squares means for UASMS1, E2JW, and EXON2-FB genotypes and breeds with the corresponding significance levels of the Wald F-tests for LEANYL, FATYL, and GFAT.

TABLE 17
Association of SNP in the Leptin gene with lean yield (LEANYL), fat
yield (FATYL) and grade fat (GFAT) in the beef cattle population

| | | Trait | | |
|---|---|---|---|---|
| | | FATYL (%) | GFAT (mm) | LEANYL (%) |
| Polygenic heritability: | | 0.62 ± 0.14 | 0.45 ± 0.15 | 0.52 ± 0.14 |
| SNP | Genotype | Least squares means^{a} | | |
| | AT | 22.4 ± 0.69 a | 8.9 ± 0.59 a | 58.6 ± 0.90 a |
| E2JW | AA | 23.9 ± 0.87 b | 10.1 ± 0.46 b | 56.8 ± 0.71 b |
| | P > F | 0.010* | 0.006* | 0.003* |
| | CC | 21.8 ± 1.00 a | 8.7 ± 0.69 a | 59.1 ± 1.04 a |
| EXON2-FB | CT | 24.0 ± 0.78 b | 10.2 ± 0.54 b | 57.1 ± 0.81 b |
| | TT | 23.7 ± 0.88 ab | 9.7 ± 0.61 ab | 56.9 ± 0.91 b |
| | P > F | 0.013 | 0.016 | 0.038 |
| | CC | 22.9 ± 1.05 ab | 9.5 ± 0.73 a | 58.1 ± 1.10 a |
| UASMS1 | CT | 22.3 ± 0.84 a | 9.1 ± 0.57 a | 58.3 ± 0.87 a |
| | TT | 24.4 ± 0.81 b | 10.1 ± 0.56 a | 56.7 ± 0.85 a |
| | P > F | 0.012 | 0.081 | 0.110 |
| Other effects | | Least square means | | |
| | SM | 20.1 ± 1.12 | 8.2 ± 0.77 | 59.1 ± 1.16 |
| Breed | LM | 22.4 ± 1.33 | 9.4 ± 0.87 | 59.9 ± 1.34 |
| | CH | 23.3 ± 1.26 | 9.4 ± 0.86 | 58.3 ± 1.30 |
| | AN | 26.9 ± 1.02 | 11.2 ± 0.68 | 53.5 ± 1.04 |
| | P > F^{b} | 0.011 | 0.006 | 0.009 |
| Sex | P > F | 0.273 | 0.705 | 0.613 |
| Slgr^{c} | P > F | 0.000 | 0.000 | 0.000 |

^{a}Means followed by different letters are significantly different (P < 0.05)
^{b}Test for the regression coefficients on the breed composition. The largest Walt F test is shown.
^{c}Slgr is the effect of slaughter group.
* Significant effect of genotype after modified Bonferroni correction for multiple tests at P = 5%.

The same table also presents the estimated polygenic heritability for the traits. The heritabilities for FATYL, LEANYL, and GFAT were moderate to high (0.62, 0.52, and 0.45, respectively), which are in agreement with expected values from literature (Utrera et al., Genet Mol Res. 2004 Sep. 30; 3(3):380-94).

For E2JW, there were only two animals with genotype TT, which were excluded from the analyses. Therefore, only solutions for genotypes AA and AT were obtained. The T allele was associated with less FATYL and GFAT and more LEANYL when compared to the A allele. The estimated differences between the heterozygote and homozygote genotypes were −1.5%, −1.2 mm and 1.9% for FATYL, GFAT, and LEANYL, respectively (P<0.05 for all differences), corresponding to 0.29, 0.36, and 0.38 phenotypic SD of the corresponding traits, respectively.

For EXON2-FB, the C allele was associated with less FATYL and GFAT and more LEANYL when compared to the T allele. The estimated differences between the homozygote genotypes CC and TT were −1.9%, −1.0 mm and 2.3% for FATYL, GFAT, and LEANYL (P=0.09, P=0.19 and P=0.05), respectively. The heterozygote genotype had, however, similar FATYL, LEANYL, and GFAT to the homozygote TT genotype, indicating a large degree of dominance of T over C allele. Differences of the CC genotype and the heterozygote genotype were all significant (P<0.05) and correspond to 0.43, 0.44, and 0.40 phenotypic SD of the corresponding traits, respectively.

For UASMS1, the C allele was associated with less FATYL than the T allele with the estimated difference between the homozygote genotypes CC and TT equal to −1.5% (P<0.05). The heterozygote genotype had similar FATYL as the homozygote CC genotype, indicating a large degree of dominance of C over T allele. There was a trend (P<0.15) that C allele might be associated with less GFAT and more LEANYL compared to T. The estimated differences between the homozygote genotypes CC and TT were −0.6 mm and 1.4% for GFAT and LEANYL, respectively.

Differences between genotypes for E2JW were also significant considering the modified Bonferroni correction for multiple tests, which was not the case for EXON2-FB and UASMS, indicating stronger evidence for the association of E2JW genotypes with FATYL, GFAT, and LEANYL than for EXON2-FB and UASMS.

Table 17 shows that there was a significant effect of breed on FATYL, GFAT, and LEANYL. Angus was the fattest breed and with the least LEANYL. Simmental had the lowest FATYL and GFAT followed by Limousin and Charolais. However, Limousin showed the highest LEANYL. There was no significant effect of sex on FATYL, GFAT and LEANYL, likely because this effect was partially confounded with slaughter group, which had a highly significant effect (Table 17).

The analyses of LM shear force in each particular postmortem day showed that the joint E2JW.EXON2-FB genotypes had a significant effect on tenderness. Table 18 presents the least squares means for the E2JW.EXON2-FB genotypes, with the corresponding significance levels for the Wald F tests.

TABLE 18
Association of SNP in the Leptin gene with tenderness (Longissimus muscle (LM) shear
force) in the beef cattle population measured at different postmortem days (2, 7, 14 and 21 days)
Trait

| | | LM2 | N | LM7 | N | LM14 | N | LM21 | N |
|---|---|---|---|---|---|---|---|---|---|
| Polygenic h² | | 0.37 ± 0.14 | 720 | 0.09 ± 0.10 | 884 | 0.39 ± 0.14 | 883 | 0.14 ± 0.10 | 877 |
| SNP | | | | | | | | | |
| E2JW × EXON2- | | Least Squares Means^{a} | | | | | | | |
| FB | | | | | | | | | |
| AA.CC | | 5.03 ± 0.46a | 240 | 4.48 ± 0.28a | 299 | 3.91 ± 0.26ab | 298 | 3.56 ± 0.20a | 296 |
| AA.CT | | 5.20 ± 0.39a | 328 | 4.57 ± 0.23a | 394 | 3.81 ± 0.21a | 394 | 3.60 ± 0.17a | 390 |
| AA.TT | | 5.56 ± 0.45a | 105 | 5.03 ± 0.28a | 126 | 4.46 ± 0.26b | 126 | 3.62 ± 0.20° | 126 |
| AT.CC | | NA | 0 | NA | 1 | NA | 1 | NA | 1 |
| AT.CT | | 5.00 ± 0.45a | 31 | 4.41 ± 0.27a | 41 | 3.92 ± 0.26a | 41 | 3.42 ± 0.21a | 41 |
| AT.TT | | 6.98 ± 0.53b | 16 | 5.53 ± 0.34b | 24 | 4.97 ± 0.31b | 24 | 4.11 ± 0.25a | 24 |
| | P > F | 0.005 | * | 0.054 | | 0.009 | * | 0.085 | |
| Other effects | | | | | | | | | |
| Breed^{b} | P > F | 0.033 | | 0.039 | | 0.176 | | 0.130 | |
| Sex | P > F | 0.254 | | 0.861 | | 0.763 | | 0.165 | |
| Slgr^{c} | P > F | 0.000 | | 0.000 | | 0.000 | | 0.000 | |

^{a}Means followed by different letters within the same genotype for E2JW are significantly different (P < 0.05)
^{b}Test for the regression coefficients on the breed composition. The largest Wald F test is shown.
^{c}Slgr is the effect of slaughter group.
* Significant effect of genotype after modified Bonferroni correction for multiple tests at P = 5%

Genotype AT.TT was significantly associated with tougher LM. Differences in shear force between genotypes AT.TT and AT.CT were substantial (1.98 kg, 1.12 kg, 1.05 kg and 0.69 kg for LM2, LM7, LM14, and LM21, respectively). Estimates for genotype AT.CC were not obtained, because there was only one animal with this genotype. Differences in shear force between genotypes AA.TT and AA.CT were smaller and mostly non significant. The magnitude of the differences between AT.TT vs. AT.CT and AA.TT vs. AA.CT genotypes illustrates the interaction between the E2JW and EXON2-FB SNP, where a larger difference exists for the AT E2JW SNP genotype. Estimates for genotypes AA.CT and AA.CC were not significantly different. Table 18 also gives the estimated polygenic heritabilities, which for LM7 and LM21 were lower than for other postmortem days.

### Example 12

### Effects of SNP Genotypes on Tenderness

Results for the average shear force over the four postmortem measures (LMAVG) are shown in Table 19.

TABLE 19
Association of SNP in the Leptin gene with average tenderness of Longissimus muscle
(LM) across different postmortem days (LMAVG) and with the intercept (LMIN) and
slope (LMSL) of the regression of tenderness measurements on days postmortem
Trait

| | LMAVG | N | LMIN | N | LMSL | N |
|---|---|---|---|---|---|---|
| Polygenic h2: | 0.42 ± 0.15 | 716 | 0.30 ± 0.13 | 884 | 0.08 ± 0.10 | 884 |

SNP

| E2JW × EXON2-F | | Least Squares Means^{a} | | | | | |
|---|---|---|---|---|---|---|---|
| AA.CC | | 4.19 ± 0.27a | 239 | 4.91 ± 0.36a | 299 | −0.064 ± 0.017a | 99 |
| AA.CT | | 4.28 ± 0.23a | 325 | 5.09 ± 0.29a | 394 | −0.078 ± 0.014a | 394 |
| AA.TT | | 4.53 ± 0.26a | 105 | 5.85 ± 0.35b | 126 | −0.106 ± 0.017a | 126 |
| AT.CC | | NA | 0 | NA | 1 | NA | 1 |
| AT.CT | | 4.04 ± 0.26a | 31 | 5.00 ± 0.36a | 41 | −0.076 ± 0.018a | 41 |
| AT.TT | | 5.36 ± 0.31b | 16 | 6.59 ± 0.43b | 24 | −0.123 ± 0.021a | 24 |
| P > F | | 0.001 | * | 0.007 | * | 0.205 | |
| Other effects | | Least Squares Means | | | | | |
| | SM | 5.43 ± 0.34 | | 7.28 ± 0.50 | | −0.16 ± 0.02 | |
| Breed | LM | 4.60 ± 0.37 | | 5.82 ± 0.54 | | −0.10 ± 0.03 | |
| | CH | 4.50 ± 0.36 | | 5.45 ± 0.54 | | −0.10 ± 0.03 | |
| | AN | 4.84 ± 0.32 | | 6.12 ± 0.46 | | −0.12 ± 0.02 | |
| | P > F^{b} | 0.112 | | 0.046 | | 0.127 | |
| Sex | P > F | 0.105 | | 0.763 | | 0.942 | |
| Slgr^{c} | P > F | 0.000 | | 0.000 | | 0.000 | |

^{a}Means followed by different letters within the same genotype for E2JW are significantly different (P < 0.05)
^{b}Test for the regression coefficients on the breed composition. The largest Wald F test is shown.
^{c}Slgr is the effect of slaughter group.
* Significant effect of genotype after modified Bonferroni correction for multiple tests at P = 5%

In addition to the genotypes' least squares means, the means for breeds are also presented. Results for E2JW.EXON2-FB genotypes were in line with those found within the different postmortem days, with the genotype AT.TT having the average toughest LM over the entire postmortem period.

A linear regression of the repeated measures of shear force on postmortem days for each animal was estimated and the individual intercepts (LMIN) and slopes (LMSL) were analyzed by the model (1). Results presented in Table 19 show a significant effect of E2JW.EXON2-FB genotypes on the intercept, but not on the slope, indicating that E2JW.EXON2-FB genotypes did not influence the effect of aging on beef tenderization. The heritabilities of the slope and intercept of LM tenderness on aging times (Table 19) indicate that the intercept is moderately heritable, whereas the slope has low heritability. A breed effect on tenderness is also shown in Table 19), showing Simmental having the toughest LM. Slaughter group had a highly significant effect on tenderness and sex effect was not significant.

Assuming either the estimated allele frequencies or equal allele frequencies (p=q=0.05) for EXON2-FB, E2JW and UASMS, and using the estimated additive (a=½ Homozygote genotype 1−½ Homozygote genotype 2) and dominance deviation (d=Heterozygote genotype−[½ Homozygote genotype 1+½ Homozygote genotype 2]) effects for the alleles, the percentage of phenotypic variation explained by each polymorphism was calculated using standard formula (FALCONER, D. S., and T. F. C. MACKAY, 1996 Introduction to Quantitative Genetics, Ed 4. Longmans Green, Harlow, Essex, UK): % V=100*(2pq [a+d(q−p)]²+[2pqd]²)/σ²ₚ), where % V is the percentage of phenotypic variation explained by the polymorphism and σ²ₚ is the phenotypic variance of the trait. For E2JW, as the TT genotype effect was not estimated, it was assumed either that the T allele shows complete domimance over C allele or the T allele has additive effect only.

Assuming equal allele frequency, EXON2-FB explained 3.2%, 3.3%, 3.2% and 23.2% of phenotypic variance for FATYL, GFAT, LEANYL, and LMAVG, respectively (Table 20).

TABLE 20
Estimated percentage of the phenotypic variation explained by the SNP
in the Leptin gene (E2JW, EXON2-FB and UASMS1) for lean yield
(LEANYL), fat yield (FATYL), grade fat (GFAT) and average tenderness
of Longissimus muscle across 21-days postmortem period (LMAVG)
Trait

| SNP | | | | | |
|---|---|---|---|---|---|
| E2JW^{c} | Allelic freq.^{a} | FATYL | GFAT | LEANYL | LMAVG^{b} |
| Dominant | Estimated | 0.62 | 0.92 | 0.92 | 3.15 |
| | 50% | 1.61 | 2.38 | 2.40 | 8.17 |
| Additive | Estimated | 0.66 | 0.97 | 0.98 | 3.35 |
| | 50% | 4.29 | 6.34 | 6.40 | 21.80 |
| EXON2- | Estimated | 3.84 | 3.93 | 3.72 | 18.42 |
| FB | | | | | |
| | 50% | 3.21 | 3.30 | 3.19 | 23.18 |
| UASMS1 | Estimated | 3.57 | ns^{d} | ns | ns |
| | 50% | 2.81 | ns | ns | ns |

^{a}Two allelic frequencies were considered into the calculations: either the estimated frequencies or the same frequency for both alleles (50%)
^{b}Calculations for LMAVG were made using the main effects of E2JW and EXON2-FB.
^{c}For E2JW, because no genotype TT effect was estimated, it was assumed either the T allele having a complete dominant effect over A allele or having an additive effect only.
^{d}Effect of UASMS1 was not significant on GFAT, LEANYL, and LMAVG.

Similarly E2JW explained 4.3%, 6.3%, 6.4% and 21.8%, and 1.6%, 2.4%, 2.4% and 8.2%, when either additive or complete dominance effects of T allele were assumed. UASMS1 explained 2.8% of phenotypic variance for FATYL. As shown in Table 20, the percentage of the phenotypic variation explained by E2JW was much smaller if the observed allele frequencies were used in the calculations, because the T allele is rare in the population. As the polygenic heritabilities for FATYL, GFAT, LEANYL, and LMAVG were around 50% (from 42% to 62%), the percentage of the total genetic variance explained by each SNP would be roughly 2-folds that in relation to the phenotypic variance.

### Example 13

### Haplotype Analyses

The linear effect of 10 haplotypes was estimated. There were three highly frequent haplotypes in the beef population (88% of all haplotypes), whose effects did not differ for any trait analyzed, even though they differ with respect to the alleles in all SNP, but E2JW. This may indicate an effect of another SNP linked to the four SNPs or some degree of epistasis among the SNP within the same chromosome. The average effect of the three common haplotypes was used as a control and all other haplotypes were contrasted against this average.

Differences in allele effects within each SNP were obtained through a linear contrast of haplotype solutions, which differed by only one allele at a given SNP. Haplotype 10 was not used in these contrasts, because it comprised the joint effect of several rare haplotypes.

Haplotypes did not significantly differ from the most frequent haplotypes and did not show any significant differences for the alleles within SNP for LMA, BONEYL, CF, HCW, ST7, and QG (data not shown), in line with the genotype analyses. For FATYL, GFAT and LEANYL, the effect of haplotype 7 (CCTT) was significantly different from the three most frequent haplotypes in the population as shown in Table 21.

TABLE 21
Association of haplotypes for SNP in the Leptin gene with lean yield (LEANYL),
fat yield (FATYL) and grade fat (GFAT) in the beef cattle population
Trait

| | | | FATYL (%) | GFAT (mm) | LEANYL (%) |
|---|---|---|---|---|---|
| Polygenic heritability: | | | 0.61 ± 0.14 | 0.43 ± 0.14 | 0.54 ± 0.14 |
| Most frequent haplotypes | | | Least Squares Mean | | |
| 1 | | | 25.19 ± 1.03 ^{a} | 10.79 ± 0.71 ^{a} | 55.79 ± 1.07 ^{a} |
| 2 | | | 25.05 ± 1.03 ^{a} | 10.52 ± 0.71 ^{a} | 56.16 ± 1.07 ^{a} |
| 3 | | | 25.30 ± 1.05 ^{a} | 10.83 ± 0.72 ^{a} | 55.77 ± 1.09 ^{a} |
| Haplotype contrasts | | | Estimate | | |
| 7 − ⅓(1 + 2 + 3) | | | −2.26 ± 0.84 | −1.84 ± 0.58 | 2.42 ± 0.87 |
| | | P > T ^{a} | 0.007 * | 0.002 * | 0.006 * |
| Allele contrasts | | SNP | Estimate | | |
| ½(7 + 8) − ½(2 + 6) | A-T | E2JW | 2.07 ± 0.81 | 1.41 ± 0.56 | −2.87 ± 0.85 |
| | | P > T | 0.011 | 0.012 | 0.001 * |
| ⅓(4 + 3 + 1) − ⅓(2 + 6 + 5) | C-T | EXON2-FB | −1.77 ± 0.85 | −1.02 ± 0.59 | 1.71 ± 0.89 |
| | | P > T | 0.036 | 0.086 | 0.053 |
| ⅓(4 + 9 + 2) − ⅓(1 + 6 + 5) | C-T | UASMS1 | −1.29 ± 0.99 | −0.91 ± 0.69 | 0.89 ± 1.03 |
| | | P > T | 0.192 | 0.186 | 0.388 |
| ⅓(5 + 1 + 2) − ⅓(6 + 3 + 9) | C-T | UASMS2 | 0.12 ± 0.73 | 0.47 ± 0.51 | 0.63 ± 0.76 |
| | | P > T | 0.872 | 0.358 | 0.414 |

^{a} Significance level of the T-test.
* Significant effect of genotype after modified Bonferroni correction for multiple tests at P = 5%

Replacing the three most frequent haplotypes by haplotype 7 would significantly decrease FATYL and GFAT by −2.26% and −1.84 mm, respectively and increase LEANYL by +2.42%, corresponding to 0.44, 0.55, and 0.48 phenotypic SD of the corresponding traits, respectively.

Table 21 also shows estimates of differences in allele effects within each SNP. In agreement with the genotype analyses, the T allele for E2JW decreased FATYL and GFAT and increased LEANYL compared to the A allele. With respect to EXON2-FB, the results were also in agreement with the genotype analyses, where the C allele decreased FATYL and GFAT and increased LEANYL compared to the T allele.

Differences in allele effects on FATYL, GFAT, and LEANYL for UASMS were all non significant. Nevertheless, the genotype analyses showed that UASMS1 genotypes significantly influenced FATYL. The contrast between haplotype effects is, however, estimating only the additive linear effect of the alleles.

For Longissimus muscle shear force at 2 and 14 days postmortem and for the average shear force over the 21 days postmortem the effect of haplotype 8 (TTTT) was significantly different from the three most frequent haplotypes in the population as shown in Table 22.

TABLE 22
Association of haplotypes for SNP in the Leptin gene with
Longissimus muscle shear force in the beef cattle population
Trait^{a}

| | | | LM2 (kg) | LM14 (kg) | LMAVG (kg) |
|---|---|---|---|---|---|
| Polygenic heritability: | | | 0.37 ± 0.14 | 0.37 ± 0.13 | 0.39 ± 0.15 |
| Most frequent haplotypes | | | Least Squares Mean | | |
| 1 | | | 4.87 ± 0.52 a | 4.06 ± 0.28 a | 3.84 ± 0.31 a |
| 2 | | | 4.72 ± 0.51 a | 4.02 ± 0.28 a | 3.90 ± 0.30 a |
| 3 | | | 4.77 ± 0.52 a | 3.93 ± 0.28 a | 3.93 ± 0.30 a |
| Haplotype contrasts | | | Estimate | | |
| 8 − ⅓(1 − 2 − 3) | | | 1.06 ± 0.41 | 0.58 ± 0.28 | 0.55 ± 0.24 |
| | | P > T^{b} | 0.009* | 0.037 | 0.021 |
| Allele contrasts | | SNP | Estimate | | |
| ½(7 + 8) − ½(2 + 6) | A-T | E2JW | −0.72 ± 0.33 | −0.36 ± 0.22 | −0.40 ± 0.20 |
| | | P > T | 0.031 | 0.096 | 0.041 |
| ⅓(4 + 3 + 1) − ⅓(2 + 6 + 5) | C-T | EXON2- | 0.24 ± 0.42 | −0.15 ± 0.23 | 0.09 ± 0.25 |
| | | FB | | | |
| | | P > T | 0.559 | 0.502 | 0.719 |
| ⅓(4 + 9 + 2) − ⅓(1 + 6 + 5) | C-T | UASMS1 | 0.30 ± 0.47 | −0.13 ± 0.27 | 0.10 ± 0.28 |
| | | P > T | 0.527 | 0.633 | 0.711 |
| ⅓(5 + 1 + 2) − ⅓(6 + 3 + 9) | C-T | UASMS2 | 0.21 ± 0.32 | 0.02 ± 0.20 | 0.10 ± 0.19 |
| | | P > T | 0.514 | 0.933 | 0.586 |

^{a}LM2, LM14 and LMAVG = Longissimus muscle shear force at 2 and 14 days postmortem and average shear force over 21 d postmortem period, respectively.
^{b}Significance level of the T-test.
*Significant effect of genotype after modified Bonferroni correction for multiple tests at P = 5%

Replacing the three most frequent haplotypes by the haplotype 8 significantly increases LM2, LM14 and LMAVG by 1.06 kg, 0.58 kg and 0.55 kg, corresponding to 0.62, 0.46 and 0.53 phenotypic SD of the corresponding measurements, respectively.

Table 22 also shows estimates of differences in allele effects within each SNP. In agreement with the genotype analyses, the T allele for E2JW increased toughness compared to the A allele. There were no significant differences between alleles for EXON2-FB. The genotype analysis showed a significant interaction between the E2JW and EXON2-FB SNP, which is not accounted for when estimating allele effects by contrasts between haplotype effects. With respect to the other two SNP, allele differences were also non significant.

The intercept and slope of individual regressions of shear force measurements on days postmortem showed that the effect of haplotype 8 (TTTT) was significantly different from the effect of the most frequent haplotypes for the intercept (0.96 kg±0.39 kg) and that there was a significant difference between the alleles T and A (0.57 kg±0.30 kg) for E2JW, but not for the other SNP. There were no significant differences in the haplotype effects on the slope of the regressions.

### Example 14

### Impact of Leptin, and Growth Hormone Receptor Genotypes on Milk Production, Feed Intake and Body Energy Traits in Dairy Cattle

Blood was collected from 571 Holstein cows in Scotland that had calved between 1991 and 2000 and participated in feed and selection trials where they had been grouped according to diet (high vs. low concentrates) and genetic merit (sired by high vs. average merit bulls).

Blood samples were used to determine genotypes for the leptin, leptin receptor and growth hormone receptor gene loci. SNPs were determined as shown in Table 2, with 6 SNPs used to define the leptin gene and one for the growth hormone receptor genes.

TABLE 23
Frequency of gene and single nucleotide polymorphisms (SNPs).

| Locus | SNP location | Polymorphisms (frequency) | | |
|---|---|---|---|---|
| Leptin | C207T | TT | CT | CC |
| | (UASMS1) | (41%) | (46%) | (13%) |
| | C528T | CC | CT | TT |
| | (UASMS2) | (82%) | (17%) | (1%) |
| | A252T | AA | AT | TT |
| | (E2JW) | (95%) | (5%) | (0%) |
| | A1457G | AA | AG | GG |
| | | (27%) | (51%) | (22%) |
| | C963T | CC | CT | TT |
| | | (42%) | (45%) | (13%) |
| | C305T | CC | CT | TT |
| | (EXON2-FB) | (43%) | (45%) | (12%) |
| Growth Hormone | F279Y | TT | AT | AA |
| Receptor | | (63%) | (34%) | (3%) |

Allelic frequencies were calculated from genotypic frequencies, and chi-square tests showed that frequencies for all loci were consistent with Hardy-Weinberg equilibrium (P>0.05). Genotypes in the 6 individual SNPs in the leptin gene were combined to produce leptin genotypes. Data were restricted to those cases where all 6 SNPs were available and cows with unique genotypes were removed from subsequent analyses. A total of 7 haplotypes were identified by parsimony. Genotypes of the leptin gene and the corresponding haplotypes are shown in Table 24 below. In this subset of data, C963T and C305T were in complete disequilibrium.

TABLE 24

Leptin genotypes and number of cows, individual
SNP order follows that of Table 1.

| | | | |
|---|---|---|---|
| Genotype | | Haplotypes | |
| code | Base sequence | involved | No. cows |

| | | | |
|---|---|---|---|
| G1 | TT CC AA AA CC CC | TCAACC × | 76 |
| | | TCAACC | |
| G2 | TT CT AA AA CC CC | TCAACC × | 42 |
| | | TTAACC | |
| G3 | CT CC AA AG CT CT | CCAGTT × | 145 |
| | | TCAACC | |
| G4 | CC CC AA GG TT TT | CCAGTT × | 54 |
| | | CCAGTT | |
| G5 | CT CC AA GG CT CT | CCAGTT × | 36 |
| | | TCAGCC | |
| G6 | TT CC AA AG CC CC | TCAACC × | 55 |
| | | TCAGCC | |
| G7 | CT CT AA AG CT CT | CCAGTT × | 28 |
| | | TTAACC | |
| G9 | CC CC AT GG TT TT | CCAGTT × | 9 |
| | | CCTGTT | |
| G10 | TT CT AA AG CC CC | TCAGCC × | 8 |
| | | TTAACC | |
| G11 | CT CC AT AG CT CT | TCAACC × | 7 |
| | | CCTGTT | |
| G12 | TT CC AA GG CC CC | TCAGCC × | 9 |
| | | TCAGCC | |
| G13 | CT CC AT GG CT CT | TCAGCC × | 6 |
| | | CCTGTT | |
| G16 | TT TT AA AA CC CC | TTAACC × | 7 |
| | | TTAACC | |

Cow genotypes were matched to individual cow production files. In the first instance, 5 production and feed intake traits were defined: daily milk yield (MY), daily fresh feed intake (FI), daily dry matter intake (DMI), daily feed over milk ratio (FMR) and daily dry matter over milk ratio (DMMR). The last two traits offer measures of feed conversion to milk. In addition, 4 body energy traits were defined: weekly live weight (LW), weekly body condition score (BCS), weekly energy content (EC) and cumulative effective energy balance (CEEB). Energy content and CEEB were based on LW and BCS records (Banos et al., 2006). Energy content was a measure of the actual energy level of the cow on day of recording. Cumulative effective energy balance was a measure of the change in energy status as it accumulates since the onset of lactation (calving day). Table 25 below summarizes these traits.

TABLE 25
Summary and descriptive statistics of production, feed intake and body energy.

| | | Frequency | | | | Mean | |
|---|---|---|---|---|---|---|---|
| | Measurement | of | No. | No. | No. | (standard | |
| Trait | unit | recording | records | cows | lactations | deviation) | |
| MY | kg | daily | 95,249 | 376 | 3 | 28.01 | (8.06) |
| FI | kg | daily | 95,249 | 376 | 3 | 51.51 | (12.61) |
| DMI | kg | daily | 95,249 | 376 | 3 | 17.88 | (4.39) |
| FMR | kg/kg | daily | 95,249 | 376 | 3 | 1.97 | (0.71) |
| DMMR | kg/kg | daily | 95,249 | 376 | 3 | 0.68 | (0.22) |
| LW | kg | weekly | 11,209 | 321 | 1 | 555.09 | (51.62) |
| BCS | score (1-5) | weekly | 11,209 | 321 | 1 | 2.69 | (0.32) |
| EC | MJ | weekly | 11,209 | 321 | 1 | 4786.98 | (699.08) |
| CEEB | MJ | weekly | 11,209 | 321 | 1 | 142.07 | (933.37) |

Statistical analyses. Each production trait was analyzed with a model including the fixed effects of selection line, diet group, lactation number, age at calving, date of calving, percentage of Holstein genes and genotype for each of the three genes (leptin, 12 d.f.; leptin receptor, 1 d.f.; and growth hormone receptor, 1 d.f.). Genetic relationships among cows were included in the analytical model and also included a 4^{th} order random regression effect of cow on day of lactation. Body energy traits were analyzed with a similar model minus lactation number plus the fixed effect of milk yield on the day of measurement.

TABLE 26
Summary and descriptive statistics of production, feed intake and body energy.

| | | Frequency | | | | Mean | |
|---|---|---|---|---|---|---|---|
| | Measurement | of | No. | No. | No. | (standard | |
| Trait | unit | recording | records | cows | lactations | deviation) | |
| MY | kg | daily | 95,249 | 376 | 3 | 28.01 | (8.06) |
| FI | kg | daily | 95,249 | 376 | 3 | 51.51 | (12.61) |
| DMI | kg | daily | 95,249 | 376 | 3 | 17.88 | (4.39) |
| FMR | kg/kg | daily | 95,249 | 376 | 3 | 1.97 | (0.71) |
| DMMR | kg/kg | daily | 95,249 | 376 | 3 | 0.68 | (0.22) |
| LW | kg | weekly | 11,209 | 321 | 1 | 555.09 | (51.62) |
| BCS | score (1-5) | weekly | 11,209 | 321 | 1 | 2.69 | (0.32) |
| EC | MJ | weekly | 11,209 | 321 | 1 | 4786.98 | (699.08) |
| CEEB | MJ | weekly | 11,209 | 321 | 1 | 142.07 | (933.37) |

Cows with the “best” leptin genotype for milk (G9) in this study produced 7.3 kg more milk per day compared to animals with the “worst” genotype (G10). Interestingly, the latter is the genotype with the best estimate of BCS and EC but worst estimate of CEEB suggesting that such cows may reach high levels of body condition and energy content but fail to maintain them; therefore, they produce less milk. The biggest “best”-“worst” difference observed in this study was for BCS (1.66 standard deviations) whilst the corresponding difference for MY amounted to 0.91 standard deviations. In general, haplotypes CCAGTT, TCAGCC and CCTGTT (following the SNP order of Table 23) appeared to be associated with high milk production. However, haplotype TTAACC was also associated with poor body condition, perhaps as a result of its tendency to utilize feed primarily for milk production.

The SNP within the growth hormone receptor gene significantly (P<0.05) affected FI, DMI, FMR, DMMR and CEEB but not MY. AA genotypes required 0.34±0.12 and 0.08±0.03 kg less fresh feed and dry matter, respectively, to produce 1 kg of milk, and also accumulated 206±96 MJ more CEEB compared to TT genotypes. The leptin receptor affected significantly (P<0.05) only DMI, with the heterozygote CT genotypes consuming 3.0±1.4 kg less dry matter than the CC homozygote (the TT homozygote was very rare).

TABLE 27
Comparison between leptin genotypes (see Table 3 for genotype codes).

| | Measurement | “Best” | “Worst” | “Best” − | “Best” − |
|---|---|---|---|---|---|
| Trait | unit | genotype | genotype | “Worst” | “Common” (G3) |
| MY* | Kg | G9 | G10 | 7.3 ± 2.4 | 4.2 ± 1.9 |
| FI | Kg | G9 | G16 | 13.4 ± 4.8 | 11.6 ± 3.4 |
| DMI | Kg | G9 | G10 | 2.6 ± 1.1 | 2.2 ± 0.9 |
| FMR | kg/kg | G16 | G10 | −0.52 ± 0.25 | −0.33 ± 0.21 |
| DMMR | kg/kg | G13 | G10 | −0.11 ± 0.07 | −0.06 ± 0.06 |
| LW | Kg | G9 | G16 | 50.9 ± 36.7 | 37.2 ± 22.7 |
| BCS* | score (1-5) | G10 | G16 | 0.53 ± 0.22 | 0.24 ± 0.15 |
| EC | MJ | G10 | G16 | 1144 ± 543 | 655 ± 386 |
| CEEB | MJ | G13 | G10 | 507 ± 186 | 188 ± 117 |

*indicates statistically significant overall effect (P < 0.05).

The results indicate the gene loci studied here may affect some economically important traits of dairy cattle (milk yield, feed and dry matter intake, feed conversion, body condition score and cumulative energy balance).

### Example 15

### Association of the SNP Marker F279Y of bGHr with the Traits in Dairy Cattle

TABLE 28
Association of the SNP marker F279Y of bGHr with the
traits yield grade, carcass weight and ribeye area^{a}.

| | | | | | | | | LSM | Number |
|---|---|---|---|---|---|---|---|---|---|
| Trait | Marker | FValue | ProbF | Genotype | Estimate | StdErr | LSM | StdErr | Obs. |
| | bGHr | 3.17 | 0.04 | AA | 0.38 | 0.21 | 3.19 | 0.21 | 1292 |
| YG | | | | AT | 0.11 | 0.06 | 2.92 | 0.07 | 1292 |
| | | | | TT | | | 2.81 | 0.04 | 1292 |
| | bGHr | 3.82 | 0.02 | AA | 50.66 | 23.44 | 741 | 23.66 | 1294 |
| CWT | | | | AT | −9.70 | 6.41 | 680 | 7.20 | 1294 |
| | | | | TT | | | 690 | 3.98 | 1294 |
| | bGHr | 5.79 | 0.00 | AA | −0.08 | 0.41 | 12.08 | 0.41 | 1293 |
| REA | | | | AT | −0.38 | 0.11 | 11.78 | 0.13 | 1293 |
| | | | | TT | | | 12.16 | 0.08 | 1293 |

^{a}the data is combined for four breeds of cattle: Red Angus, Charelois, Brangus and Brahman.

### Example 16

### Marker and Trait Associations

TABLE 29
Carcass traits

| | | | EXON2- | | |
|---|---|---|---|---|---|
| | UASMS1 | UASMS2 | FB | E2JW | bGHr |
| Trait | C207T | C528T | C1180T | A252T | F279Y |
| HCWT | ✓ C | ✓ C | C | | |
| MB | C | C | C | C | |
| REA | C | C | ✓ C | | ✓ |
| BFAT | ✓ C | C | ✓ C | ✓ C | ✓ |
| DP | C | ✓ C | C | | |
| QG | | | C | | |
| YG | C | C | ✓ C | | ✓ |
| Tenderness | | | ✓ C | ✓ C | |
| Fat yield | ✓ | | ✓ C | ✓ C | |
| Lean yield | | | ✓ | ✓ | |

TABLE 30
Performance traits

| | | | EXON2- |
|---|---|---|---|
| | UASMS1 | UASMS2 | FB |
| Trait | C207T | C528T | C1180T |
| ADG | ✓ C | ✓ C | C |
| DOF | C | C | C |
| Live wt | ✓ C | ✓ C | C |
| US BFAT | ✓ | ✓ | |
| US MB | ✓ | ✓ | |
| US REA | ✓ | | |
| DMI | ✓ | ✓ | |
| Residual FI | ✓ | | |

In the Tables 29 and 30 above, √ means significant association as a single marker. C means significant association in combination with one or more other markers.

### Example 17

Also evaluated were four SNPs (UASMS1, UASMS2, UASMS3, and EXON2-FB) in the leptin gene, and one SNP in the growth hormone receptor gene in a large commercial feedlot population with live-animal performance and carcass trait measurements. The polymorphisms used in this study were UASMS1, UASMS2, UASMS3 and F279Y of bGHr, the latter as described by Blott et al., (Genetics. 2003 January; 163(1):253-66) incorporated herein by reference in its entirety. This association analysis was predicated on the assumption that the markers are in linkage disequilibrium with the casual mutation that is either in the gene for which the polymorphic marker is located or a gene in close proximity to the marked gene.

The original data set consisted of 2,189 records on steer and heifer calves fed at the Decatur County Feed Yard (DCFY) in Oberlin, Kans. After forming contemporary groups, a total of 1,633 steers and heifers were available for the analysis (Table 1). Traits analyzed (with appropriate abbreviations) included: Hot carcass weight, lb (HCW); Ribeye area, in² (REA), Ribeye area per hundred weight of carcass, in²/100 lb HCW (REA/cwt), Hot carcass weight value, $ (HCW value), Calculated live weight, lb (Calc Lv Wt), Predicted total dry matter intake, lb (DMI), Days on feed, d (DOF), DMI per DOF, lb/d (DMI/DOF), Average daily gain, lb/d (ADG), Dressing percentage, % (DP), Backfat deposition rate, % (BFAT dep rate), Carcass backfat at the plant, in (BFAT), Calculated USDA yield grade (YG), USDA quality grade, (QG), Intramuscular fat percentage, % (IMF %), Marbling score (MBS), Marbling score per DOF, change in score/d (MBS/DOF), Additional carcass value, $, Adjusted net return, $, Adjusted net return with arrival costs removed, $.

Statistical Analysis. Data were analyzed with the following models, all of which included a fixed CG effect:

1) Genotype—regression on genotypes as fixed effects,

2) Allele substitution—regression on allele number (0, 1, 2) when looking at single marker loci, and

3) Haplotype—regression on haplotype when fitting multiple leptin markers.

Leptin marker analyses were done individually and as genotype combinations using Model 1, and as haplotypes consisting of paired marker combinations as well as all three markers combined using Model 3. bGHr was only analyzed with Models 1 and 2.

Haplotype model estimates are deviations from the last haplotype fit in the statistical analysis software—that is why the “b” value is always set to 0.0 for one haplotype. In addition, low haplotype frequency can lead to extreme estimates that are significant; however, the biological difference between other well-represented haplotypes may not be that great.

Genotyping results indicated that UASMS1 and UASMS3 are in perfect linkage disequilibrium. Therefore, only UASMS1 results are discussed further. Genotype and allele frequencies are based on observed genotypes from 1,954 records (Table 31).

TABLE 31

Frequency

| Marker | Genotype | Number | Genotype | Allele |
|---|---|---|---|---|
| UASMS1 | CC | 380 | 19.45 | 0.43 |
| | CT | 935 | 47.85 | |
| | TT | 639 | 32.70 | 0.57 |
| UASMS2 | CC | 977 | 50.00 | 0.71 |
| | CT | 809 | 41.40 | |
| EXON2- | TT | 168 | 8.60 | 0.29 |
| FB | CC | 638 | 32.65 | 0.57 |
| | CT | 940 | 48.11 | |
| | TT | 376 | 19.24 | 0.43 |
| bGHr | AA | 21 | 1.07 | 0.10 |
| | AT | 362 | 18.53 | |
| | TT | 1571 | 80.40 | 0.90 |

The frequency of the rare allele in the leptin markers is moderate to high. In contrast, the frequency of the rare allele in the bGHr gene was very low, resulting in very few animals with the homozygous genotype for these alleles.

Results: In most cases, the models identified similar significant effects; however, there were cases where significance was observed in some but not all analyses. Numeric results for traits and markers are only presented when a significant effect (P<0.05) was detected. In some cases, those associations that approached significance, i.e., P<0.10 are presented.

Leptin Gene-Allele Substitution Model. Genotype and allele frequencies indicated these markers would be suitable for marker-assisted selection or management (Table 32). In general, markers in the leptin gene were associated with body and muscle size; all three approaching significance or significant for HCW, REA, and Calc Lv Wt (Table 32).

TABLE 32
Regression on the Number of Alleles

| | UASMS1 [C] | | | UASMS2 [C] | | |
|---|---|---|---|---|---|---|
| Marker | b | se | Prob. | b | se | Prob. |
| HCW | −5.536 | 2.221 | 0.013 | 6.650 | 2.433 | 0.006 |
| REA | −0.116 | 0.053 | 0.028 | 0.110 | 0.058 | 0.057 |
| REA/cwt | | | | | | |
| Calc Lv | −8.425 | 3.308 | 0.011 | 6.916 | 3.630 | 0.057 |
| Wt | | | | | | |
| DP | | | | 0.191 | 0.073 | 0.009 |
| BFAT | | | | | | |
| dep rate | | | | | | |
| BFAT | 0.013 | 0.005 | 0.010 | | | |
| YG | 0.050 | 0.023 | 0.030 | | | |
| MBS | | | | | | |
| | EXON2-FB [C] | | | bGHr [A] | | |
| Marker | b | se | Prob. | b | se | Prob. |
| HCW | 5.230 | 2.247 | 0.020 | −7.627 | 3.661 | 0.037 |
| REA | 0.149 | 0.054 | 0.005 | −0.352 | 0.087 | <.0001 |
| REA/cwt | | | | −0.029 | 0.012 | 0.012 |
| Calc Lv | 7.292 | 3.342 | 0.029 | | | |
| Wt | | | | | | |
| DP | | | | −0.225 | 0.109 | 0.038 |
| BFAT | 0.00004 | 0.0001 | 0.049 | 0.0004 | 0.0002 | 0.057 |
| dep rate | | | | | | |
| BFAT | −0.016 | 0.005 | 0.002 | 0.030 | 0.008 | 0.000 |
| YG | −0.072 | 0.024 | 0.002 | 0.162 | 0.038 | <.0001 |
| MBS | −0.573 | 0.319 | 0.072 | | | |

UASMS1 and EXON2-FB SNPs are also significantly associated with BFAT and YG. The “C” allele at the UASMS1 marker locus was associated with decreased body weight (BW) and REA, and slightly higher BFAT and YG. The “C” allele at EXON2-FB has exactly the opposite effect—it is associated with increased BW and REA, and slightly lower BFAT and YG, as well as a trend towards lower MBS. The UASMS2 “C” allele was associated with increased HCW and DP, and approached significance for increased REA and Calc Lv Wt.

The effects of the “C” alleles in UASMS1 and EXON2-FB found in this study were similar (relative to traits and direction of effect) to published results on these markers (Nkrumah et al., 2004; Nkrumah et al., 2005). Surprisingly, in this study, UASMS2 was found to affect BW as well but the ranking of genotypes is reversed. Haplotype Model. Tables 33 to 35 contain the results of the various combinations of two- and three-marker leptin haplotype regressions. The haplotype analysis results are similar in terms of significant effects as reported for the single marker results in most cases.

TABLE 33
Regression on Leptin Haplotype - UASMS1 & UASMS2

| | | Haplotype | | | |
|---|---|---|---|---|---|
| Trait | | C-C | C-T | T-C | T-T |
| | Frequency | 0.435 | 0.002 | 0.267 | 0.296 |
| | se | 0.008 | 0.001 | 0.007 | 0.008 |
| HCW | b | 2.709 | −77.040 | 29.078 | 0.000 |
| | se | 5.290 | 66.014 | 6.074 | . |
| | Probability | 0.609 | 0.243 | <.0001 | . |
| REA | b | 0.013 | −2.158 | 0.526 | 0.000 |
| | se | 0.126 | 1.575 | 0.145 | . |
| | Probability | 0.917 | 0.171 | 0.000 | . |
| DP | b | 0.232 | −0.742 | 0.582 | 0.000 |
| | se | 0.158 | 1.971 | 0.181 | . |
| | Probability | 0.142 | 0.707 | 0.001 | . |
| BFAT dep | b | −0.00006 | −0.00061 | −0.00079 | 0.000 |
| rate | se | 0.00027 | 0.00335 | 0.00031 | . |
| | Probability | 0.838 | 0.855 | 0.011 | . |
| BFAT | b | 0.016 | 0.332 | −0.021 | 0.000 |
| | se | 0.012 | 0.149 | 0.014 | . |
| | Probability | 0.180 | 0.026 | 0.131 | . |
| Calc Lv | b | −0.278 | −110.727 | 34.821 | 0.000 |
| Wt | se | 7.892 | 98.477 | 9.061 | . |
| | Probability | 0.972 | 0.261 | 0.000 | . |
| DOF | b | 0.439 | −22.817 | 1.100 | 0.000 |
| | se | 0.879 | 10.963 | 1.009 | . |
| | Probability | 0.617 | 0.038 | 0.276 | . |

TABLE 34
Regression on Leptin Haplotype - UASMS2 & EXON2-FB

| | | Haplotype | | | |
|---|---|---|---|---|---|
| Trait | | C-C | C-T | T-C | T-T |
| | Frequency | 0.281 | 0.421 | 0.279 | 0.018 |
| | se | 0.008 | 0.008 | 0.007 | 0.002 |
| HCW | b | 43.004 | 19.603 | 16.376 | 0.000 |
| | se | 19.516 | 19.929 | 20.223 | . |
| | Probability | 0.028 | 0.325 | 0.418 | . |
| REA | b | 1.275 | 0.706 | 0.738 | 0.000 |
| | se | 0.465 | 0.475 | 0.482 | . |
| | Probability | 0.006 | 0.138 | 0.126 | . |
| DP | b | 1.620 | 1.411 | 1.232 | 0.000 |
| | se | 0.581 | 0.593 | 0.602 | . |
| | Probability | 0.005 | 0.017 | 0.041 | . |
| BFAT dep | b | −0.00246 | −0.00168 | −0.00174 | 0.000 |
| rate | se | 0.00100 | 0.00102 | 0.00103 | . |
| | Probability | 0.014 | 0.100 | 0.093 | . |
| BFAT | b | −0.11482 | −0.07540 | −0.09830 | 0.000 |
| | se | 0.04484 | 0.04576 | 0.04646 | . |
| | Probability | 0.011 | 0.100 | 0.035 | . |
| YG | b | −0.529 | −0.339 | −0.420 | 0.000 |
| | se | 0.211 | 0.215 | 0.218 | . |
| | Probability | 0.012 | 0.116 | 0.055 | . |
| MBS | b | −6.464 | −6.151 | −7.689 | 0.000 |
| | se | 3.108 | 3.147 | 3.195 | . |
| | Probability | 0.038 | 0.051 | 0.016 | . |
| MBS/DOF | b | −0.048 | −0.045 | −0.054 | 0.000 |
| | se | 0.025 | 0.025 | 0.026 | . |
| | Probability | 0.054 | 0.078 | 0.036 | . |

TABLE 35
Regression on Leptin Haplotype - UASMS1 & EXON2-FB

| | | Haplotype | | | |
|---|---|---|---|---|---|
| Trait | | C-C | C-T | T-C | T-T |
| | Frequency | 0.025 | 0.414 | 0.535 | 0.026 |
| | se | 0.003 | 0.008 | 0.008 | 0.003 |
| REA/cwt | b | 0.134 | 0.029 | 0.044 | 0.000 |
| | se | 0.065 | 0.045 | 0.044 | . |
| | Probability | 0.040 | 0.521 | 0.319 | . |
| BFAT dep | b | −0.00112 | −0.00116 | −0.00157 | 0.000 |
| rate | se | 0.00106 | 0.00072 | 0.00072 | . |
| | Probability | 0.292 | 0.110 | 0.030 | . |
| BFAT | b | −0.071 | −0.055 | −0.089 | 0.000 |
| | se | 0.047 | 0.032 | 0.032 | . |
| | Probability | 0.132 | 0.085 | 0.006 | . |
| YG | b | −0.458 | −0.206 | −0.342 | 0.000 |
| | se | 0.218 | 0.150 | 0.150 | . |
| | Probability | 0.036 | 0.170 | 0.022 | . |

Significant associations for UASMS1 & UASMS2 are: HCW, REA, DP, BFAT dep rate, BFAT, Calc Lv Wt, and DOF (Table 33). The T-C haplotype is superior for red meat yield.

Significant associations for UASMS2 & EXON2-FB are: HCW, REA, DP, BFAT dep rate, BFAT, YG, MBS, and MBS/DOF (Table 34). The C-C haplotype is superior for red meat yield, whereas the T-T haplotype is best for cattle targeted at quality markets where increased backfat and marbling results in a premium price.

Significant associations for UASMS1 & EXON2-FB are: REA/cwt, BFAT dep rate, BFAT, and YG (Table 35). Differences between halplotyes are not as consistent for these two markers as with the other combinations.

Significant associations for UASMS1 & UASMS2 with EXON2-FB are: HCW, REA, REA/cwt, DP, BFAT dep rate, BFAT, YG, ADG, and DOF (Table 33). As might be expected based on the previous leptin results, animals with the T-C-C haplotype appear to outperform others in terms of producing high-yielding carcasses.

Growth Hormone Receptor Gene-Allele Substitution Model. The marker located in the bGHr gene was significantly associated with HCW, REA, REA/cwt, DP, BFAT, and YG (Table 32). The “T” allele is associated with increased HCW, REA, REA/cwt, and DP, and decreased BFAT and YG. Differences between homozygotes for REA and YG are sizeable and in the appropriate direction, 0.704 in² and 0.324 YG, respectively. The association between bGHr and BFAT dep rate approaches significance, with the “T” allele tending towards a lower rate of deposition. The frequency of the “T” allele in these data is very high, 0.90, suggesting producers have already been effective in selecting for the favorable allele through means other than marker-assisted selection.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method for sub-grouping animals according to genotype wherein the animals of each sub-group have a similar polymorphism or combination of polymorphisms in the leptin gene comprising:
(a) determining the genotype of each animal to be sub-grouped by determining the presence of a single nucleotide polymorphism or a combination of single nucleotide polymorphisms in polymorphic loci of the leptin (ob) gene, wherein the single nucleotide polymorphic loci are selected from the group consisting of UASMS1, UASMS2, UASMS3, EXON2-FB, and E2JW, and
(b) segregating individual animals into sub-groups wherein each animal in a subgroup has a similar polymorphism or combination of polymorphisms.

2. The method according to claim 1, wherein the animal is a bovine and the leptin gene is the bovine leptin gene.

3. The method according to claim 1, further comprising determining the presence of a single nucleotide polymorphism in the bovine growth hormone receptor.

4. The method according to claim 3, further wherein the single nucleotide polymorphism in the bovine growth hormone receptor is F279Y, and wherein F279Y is a determinant of ribeye area, yield grade and daily material intake.

5. The method according to claim 1, wherein the combination of single nucleotide polymorphisms of the leptin gene is selected from the group consisting of UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, and UASMS3/E2JW, and wherein segregating individual animals into sub-groups depends on whether the animals have, or do not have, the UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, and UASMS3/E2JW single nucleotide polymorphism combinations of the leptin gene.

6. The method according to claim 5, wherein the combination of single nucleotide polymorphisms of the leptin gene comprises the markers UASMS1/EXON2-FB, UASMS3/EXON2-FB, EXON2-FB/E2JW, UASMS1/E2JW, or UASMS3/E2JW, and wherein the combination of SNPs indicates an increase in the tenderness of meat.

7. The method according to claim 5, wherein the combination of single nucleotide polymorphisms of the leptin gene comprises the markers UASMS1/EXON2-FB, UASMS3/EXON2-FB, EXON2-FB/E2JW, UASMS1/E2JW, or UASMS3/E2JW.

8. The method according to claim 5, wherein the combination of single nucleotide polymorphisms of the leptin gene comprises the markers EXON2-FB/E2JW.

9. The method according to claim 5, wherein the combination of single nucleotide polymorphisms of the leptin gene comprises the markers UASMS1/EXON2-FB or UASMS3/EXON2-FB.

10. The method according to claim 5, wherein the combination of single nucleotide polymorphisms of the leptin gene comprises the markers UASMS1/E2JW, or UASMS3/E2JW.

11. The method according to claim 4, wherein the combination of single nucleotide polymorphisms is selected from the group consisting of UASMS1/F279Y, UASMS2/F279Y, UASMS3/F279Y, EXON2-FB/F279Y, F279Y/E2JW, UASMS1/UASMS2/F279Y, UASMS1/UASMS3/F279Y, UASMS2/UASMS3/F279Y, UASMS1/EXON2-FB/F279Y, UASMS2/EXON2-FB/F279Y, UASMS3/EXON2-FB/F279Y, EXON2-FB/E2JW/F279Y, UASMS1/E2JW/F279Y, UASMS2/E2JW/F279Y, and UASMS3/E2JW/F279Y.

12. A method for identifying an animal having a desirable phenotype relating to certain feed intake, growth rate, body weight, carcass merit and composition, and milk yield, as compared to the general population of animals of that species, comprising determining the presence of a single nucleotide polymorphism or combination of single nucleotide polymorphisms of the animal, wherein the single nucleotide polymorphism is selected from the group consisting of UASMS1, UASMS2, UASMS3, EXON2-FB, E2JW and F279Y, and wherein the combination of single nucleotide polymorphisms is selected from the group consisting of UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, UASMS3/E2JW, UASMS1/F279Y, UASMS2/F279Y, UASMS3/F279Y, EXON2-FB/F279Y, F279Y/E2JW, UASMS1/UASMS2/F279Y, UASMS1/UASMS3/F279Y, UASMS2/UASMS3/F279Y, UASMS1/EXON2-FB/F279Y, UASMS2/EXON2-FB/F279Y, UASMS3/EXON2-FB/F279Y, EXON2-FB/E2JW/F279Y, UASMS1/E2JW/F279Y, UASMS2/E2JW/F279Y, and UASMS3/E2JW/F279Y, and wherein the presence of either the UASMS1, UASMS2, UASMS3 or EXON2-FB single nucleotide polymorphism or the presence of the UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, UASMS3/E2JW, UASMS1/F279Y, UASMS2/F279Y, UASMS3/F279Y, EXON2-FB/F279Y, F279Y/E2JW, UASMS1/UASMS2/F279Y, UASMS1/UASMS3/F279Y, UASMS2/UASMS3/F279Y, UASMS1/EXON2-FB/F279Y, UASMS2/EXON2-FB/F279Y UASMS3/EXON2-FB/F279Y, EXON2-FB/E2JW/F279Y, UASMS1/E2JW/F279Y, UASMS2/E2JW/F279Y, and UASMS3/E2JW/F279Y combinations of single nucleotide polymorphisms is indicative of a desirable phenotype relating to certain feed intake, growth rate, body weight, carcass merit and composition, meat quality, meat tenderness or and milk yield.

13. A composition for the detection of a combination of single nucleotide polymorphisms selected from the group consisting of UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, UASMS3/E2JW, UASMS1/F279Y, UASMS2/F279Y, UASMS3/F279Y, EXON2-FB/F279Y, F279Y/E2JW, UASMS1/UASMS2/F279Y, UASMS1/UASMS3/F279Y, UASMS2/UASMS3/F279Y, UASMS1/EXON2-FB/F279Y, UASMS2/EXON2-FB/F279Y UASMS3/EXON2-FB/F279Y, EXON2-FB/E2JW/F279Y, UASMS1/E2JW/F279Y, UASMS2/E2JW/F279Y, or UASMS3/E2JW/F279Y, comprising at least two oligonucleotide probes, wherein each oligonucleotide probe is capable of selectively detecting a single polymorphism, and wherein each probe is optionally labeled with a detectable moiety.

14. The isolated oligonucleotide probe according to claim 10, wherein the detectable moiety is selected from the group consisting of a radiolabel ³H, ¹²⁵I, ³⁵S, ¹⁴C, ³²P, a detectable enzyme, horse radish peroxidase (HRP), alkaline phosphatase, a fluorescent dye, fluorescein isothiocyanate, Texas red, rhodamine, Cy3, Cy5, Bodipy, Bodipy Far Red, Lucifer Yellow, Bodipy 630/650-X, Bodipy R6G-X, 5-CR 6G, a colorimetric label, colloidal gold digoxigenin-dUTP, or biotin.

15. The isolated oligonucleotide according to claim 10, wherein the oligonucleotide is immobilized on a solid support.

16. A method of determining the genotype of an animal at a polymorphic locus of the ob gene comprising
a) obtaining a DNA sample from the animal
b) contacting the DNA sample with at least two oligonucleotide primer pairs under conditions suitable for permitting hybridization of the oligonucleotide primers to the DNA sample,
c) enzymatically amplifying specific regions of the ob gene using the primer pairs to form at least two nucleic acid amplification products,
d) contacting the amplification products from step c) with labeled ob gene allele-specific probes, labeled with a detectable moiety, under conditions suitable for permitting hybridization of the labeled allele-specific probes to the amplification products, and
e) detecting the presence of the amplification products by detecting the detectable moiety of the labeled allele-specific probes hybridized to the amplification products.

17. The method according to claim 16, wherein at least one oligonucleotide primer pair is selected from the group consisting of SEQ ID NO: 7 and SEQ ID NO: 8, SEQ ID NO: 11 and SEQ ID NO: 12, SEQ ID NO: 15 and SEQ ID NO: 16, SEQ ID NO: 19 and SEQ ID NO: 20 and SEQ ID NO: 21 and SEQ ID NO: 22.

18. The method according to claim 16, wherein the oligonucleotide primer pairs are capable of amplifying regions of the bovine leptin gene having at least one polymorphic nucleotide locus selected from the group consisting of UASMS1, UASMS2, UASMS3, EXON2-FB, and E2JW, or combinations thereof selected from the group consisting of UASMS1/UASMS2, UASMS1/UASMS3, UASMS2/UASMS3, UASMS1/EXON2-FB, UASMS2/EXON2-FB, UASMS3/EXON2-FB, EXON2-FB/E2JW, UASMS1/E2JW, UASMS2/E2JW, or UASMS3/E2JW.

19. The method according to claim 16, wherein the oligonucleotide primer pairs are capable of amplifying the region of the bovine growth hormone receptor (bGHr) gene having the SNP F279Y.
